(19) United States
(12) Patent Application Publication
(10) Pub. No.: US 2014/0236070 A1
(43) Pub. Date: Aug. 21, 2014

(54) LINEAR EXPRESSION CASSETTES AND USES THEREOF

(71) Applicants: Kate Broderick, San Diego, CA (US); Feng Lin, San Diego, CA (US); Niranjan Sardesai, Blue Bell, PA (US); Xuefei Shen, San Diego, CA (US)

(72) Inventors: Kate Broderick, San Diego, CA (US); Feng Lin, San Diego, CA (US); Niranjan Sardesai, Blue Bell, PA (US); Xuefei Shen, San Diego, CA (US)

(21) Appl. No.: 14/235,176

(22) PCT Filed: Jul. 27, 2012
(86) PCT No.: PCT/US12/48494

§ 371 Date: Apr. 30, 2014

Related U.S. Application Data
(60) Provisional application No. 61/513,133, filed on Jul. 29, 2011.

(51) Int. Cl. C07K 14/005; A61N 1/32; C12N 9/24

(52) U.S. Cl. CPC C07K 14/005; C12N 9/2402; A61N 1/327
USPC 604/20; 435/320.1; 604/501

(57) ABSTRACT

Provided herein are linear nucleic expression cassettes and methods of using same in a non-invasive method of vaccination. The method combines electroporation and linear DNA constructs encoding antigens to elicit antigen-specific immune responses.

### FIELD OF THE INVENTION

The present invention relates to linear expression cassettes that expresse one or more antigens, and methods of non-invasively vaccinating a subject with same linear expression cassettes.

### BACKGROUND

Many vaccines rely on the ‘predict and produce’ approach. For example, influenza vaccines are generated based on the hemagglutinin and neuraminidase sequences of virus strains that are the most likely to spread across the globe during flu season. However, changes in a circulating virus or the emergence of a pandemic strain with major changes in its glycoproteins would render such vaccines ineffective. In addition, the current egg-based influenza vaccine manufacturing technology depends on the ability of the flu strain to replicate in eggs and takes at least six months to manufacture sufficient doses for the seasonal vaccination campaign.

The production capacity for current vaccines is estimated to be lower that what is required to vaccinate the present global population. For example, the current influenza vaccine globally is estimated to be approximately 826 million seasonal influenza vaccine doses (inactivated and live) which is far less than what is required to vaccinate the global population of 6.3 billion. Further, vaccine production capacity is concentrated mostly in North America, Europe, Australia, Japan, Russia, and China. Accordingly, the capacity for increasing dose production in case of a pandemic virus outbreak for the U.S. and world population is limited and usually strain dependent.

Therefore, faster and simplified vaccine manufacturing technologies are needed for influenza and non-influenza-related vaccine strategies to be a viable solution in the event of a future pandemic. In addition, there is a need for a method to quickly and efficiently administer such vaccinations in a prophylactic setting.

### SUMMARY OF THE INVENTION

Provided herein are nucleic acid constructs comprising linear expression cassettes that express one or more antigens. The linear expression cassette is capable of expressing a desired antigen in cells of a subject, and the antigen encoding sequence can be one of, or a plurality of, nucleic acid sequences, comprising an antigen encoding sequence selected from the group consisting of SEQ ID NOs: 3, 5, 7, 9, 11, 13, 15, 17, 19, 21, 23, 25, 27, 29, 31, 33, 35, 38, 44, 46, 194, 196, 198, 200, 202, 204, 206, 208, 210, 212, 214, 216, 218, 220, 222, 224, 226, 228, 230, 232, 234, 236, 238, 240, 242, 244, 246, 248, and sequences that are at least 98% similar thereof; or antigen encoding sequence encoding an antigen selected from the group consisting of SEQ ID NOs: 1, 2, 4, 6, 8, 10, 12, 14, 16, 18, 20, 22, 24, 26, 28, 30, 32, 34, 36, 39-43, 45, 47, 48-193, 195, 197, 199, 201, 203, 205, 207, 209, 211, 213, 215, 217, 219, 221, 223, 225, 227, 229, 231, 233, 235, 237, 239, 241, 243, 245, 247, and 249, and sequences that are at least 98% similar thereof.

Preferably the antigen encoding sequence is SEQ ID NO:1 or SEQ ID NO:2.

There are also provided herein methods of non-vasive vaccination comprising the linear expression cassettes provided. The methods comprise electroporating a linear expression cassette into a subject in need thereof. The linear expression cassette (LEC) may be a nucleic acid that encodes one or more antigens. The LEC may be electroporated intradermally and/or intramuscularly. The antigen may be any antigen capable of eliciting an immune response in the subject. The antigen may be associated with influenza, human papillomavirus, hepatitis C virus, flea allergen FSA1, Der p1, type 1 diabetes mellitus, multiple sclerosis, autoimmune ovarian disease, myocarditis, rheumatoid arthritis, thyroiditis, myasthenia gravis, autoimmune uveitis, hTERT, PSA, PSMA, STEAP, PSCA, or a Foot and mouth disease virus. The antigen may be M2, LACK, HBV, neuraminidase, hemagglutinin, or a variant thereof or a consensus thereof, for example. Expression of the antigen may be driven by a promoter such as a CMV promoter, SV40 early promoter, SV40 later promoter, metallothionein promoter, murine mammary tumor virus promoter, Rous sarcoma virus promoter, or polyhedrin promoter. The LEC may be electroporated via a minimally-invasive electroporation device. The LEC may be perNP or perM2.

Also provided herein is a kit that may comprise an electroporation device and a linear expression cassette, wherein the linear expression cassette comprises a nucleic acid encoding one or more antigens as described herein. The electroporation device may be a minimally-invasive electroporation device. The LEC may be perNP or perM2.

### BRIEF DESCRIPTION OF THE DRAWINGS

FIG. 1 shows maps of plasmid expression vectors encoding influenza nucleoprotein (“NP”) and M2 antigens and the corresponding linear expression cassettes. The linear expression cassette perNP or perM2 contain CMV promoter, intron for splicing, full length gene of NP or M2 with stop codon and polyadenylation signal.

FIG. 2 shows in vitro expression of linear DNA expression cassettes. (A) HK 293 cells were transfected with mock DNA, pNP or perNP on chamber slides. 24 hours after transfection, slides or resuspended cells were stained for intracellular NP. Cells were visualized under fluorescence microscopy and fraction of NP expressing cells were analyzed by flow cytometry analysis. (B) HK 293 cells were transfected with no DNA, pM2 or lecM2 on chamber slides. 24 hours after transfection, slides or resuspended cells were stained for surface and intracellular M2. Resuspended cells were also stained for surface M2. Cells were visualized under fluorescence microscopy and fraction of M2 expressing cells were analyzed by flow cytometry analysis.

FIG. 3 shows Balb/c Mice in group of 5 were immunized once with pNP or lecNP using intradermal electroporation (“ID EP”). Two or five weeks after immunization, anti-NP responses and NP specific CTL responses were measured for each group of mice.

FIG. 4 shows Balb/c Mice in group of 10 were immunized as in Table 1. 2 week after the last immunization, (A) Anti-NP antibody responses, (B) Anti-M2e antibody responses were measured for each mice. (C) NP specific cytotoxic T lymphocytes (“CTL”) and (D) M2e specific HTL responses were measured for each group of mice. E. 10 week after the last immunization, the immunized and naive mice were challenged with 5×105 TCID/Mouse of H1N1 influenza strain A/Canada/AB/RV1532/2009 and body weight change of each mouse were monitored.

FIG. 5 shows mice in group of 10 were immunized with pNP and pM2 or equal moles of lecNP and lecM2 using IM EP or ID EP on week 0, 3, and 10. (A) On week 15, the immunized and naive mice were challenged with 100×LD50 of H5N1 influenza strain VN/1203/04 and body weights of each mouse were monitored. (B) Mortality of each mouse was monitored for three weeks after the challenge.

### DETAILED DESCRIPTION

The inventors have made the surprising discovery that linear nucleic acid vaccines can elicit antigen-specific antibody responses, which are sustainable for longer periods of time as compared to plasmid-based vaccines, when efficiently delivered by electroporation. The herein described method uses a minimally-invasive electroporation technique to deliver a linear nucleic acid vaccine (a linear expression cassette or “LEC”), which provides a longer lasting antigen-specific immune response that is well tolerated by the subject population. The present invention is also directed to a number of antigens that can be expressed from the LEC.

### 1. Definitions

The terminology used herein is for the purpose of describing particular embodiments only and is not intended to be limiting. As used in the specification and the appended claims, the singular forms “a,” “and” and “the” include plural references unless the context clearly dictates otherwise.

a. Consensus or Consensus Sequence

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes or sertypes of a particular antigen. Synthetic antigens, such as fusion proteins, may be manipulated to consensus sequences (or consensus antigens).

b. Variant

“Variant” as used herein may mean a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6,9, and 7.0 are explicitly contemplated.

### 2. Method of Vaccination

Provided herein is a method of vaccinating a subject. The method uses electroporation as a mechanism to deliver a linear nucleic acid vaccine. The electroporation may be carried out via a minimally invasive device.

a. Linear Nucleic Acid Vaccine

Provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

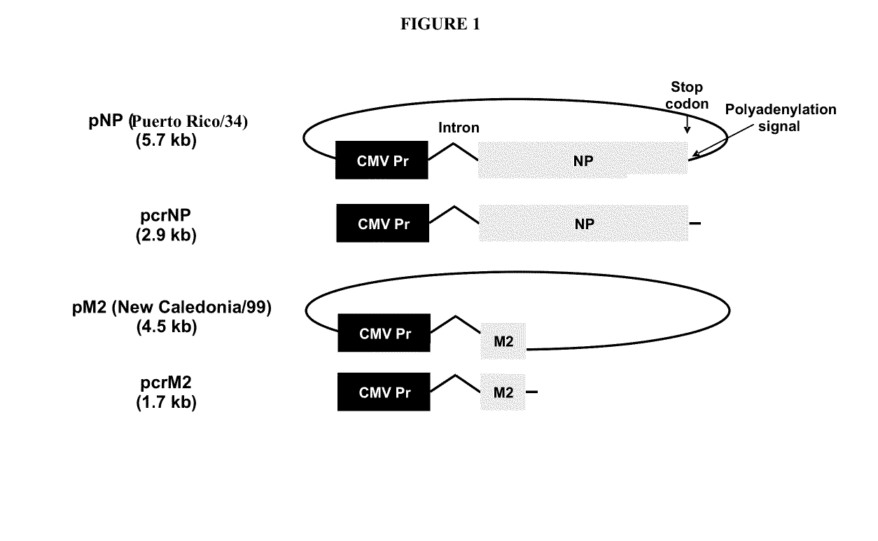

The LEC may be derived from any plasmid capable of being linearized. The plasmid may be capable of expressing the antigen. The plasmid may be pNP (Puerto Rico/34) or pM2 (New Caledonia/99). See FIG. 1. The plasmid may be pVAX, pcDNA3.0, or provax, or any other expression vector capable of expressing the DNA and enabling a cell to translate the sequence to a antigen that is recognized by the immune system.

The LEC may be perM2 as shown in FIG. 1. The LEC may be perNP as shown in FIG. 1. perNP and perMR may be derived from pNP (Puerto Rico/34) and pM2 (New Caledonia/99), respectively. See FIG. 1. The LEC may be combined with antigen at a mass ratio of between 5:1 and 1:5, or of between 1:1 to 2:1.

(1) Promotor, Intron, Stop Codon, and Polyadenylation Signal

The promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the LEC as it is from the transcription start site in its natural setting. However, variation in this distance may be accommodated without loss of promoter function.

The LEC thus contains a promoter operably linked to the nucleic acid sequence encoding the antigen and signals required for efficient polyadenylation of the transcript, ribosome binding sites, and translation termination. Additional elements of the LEC may include an enhancer and an intron with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

(2) Antigen

Provided herein is an antigen, which may be encoded by any DNA and/or RNA sequence. The antigen may be a peptide or protein that causes an immune response. The antigen may trigger the production of an antibody by the immune system. The antibody may then kill or neutralize the antigen that is recognized as a foreign and potentially harmful invader. The antigen may be any molecule or molecular fragment that can be bound by a major histocompatibility complex (MHC) and presented to a T-cell receptor. The antigen may be an immunogen, which may be a molecule that is able to provoke an adaptive immune response if injected on its own.

The antigen may be associated with influenza, autoimmune disease, human papillomavirus, hepatitis C virus, visceral leishmaniasis, type 1 diabetes mellitus, multiple sclerosis, autoimmune ovarian disease, myocarditis, rheumatoid arthritis, thyroiditis, myasthenia gravis, or autoimmune uveitis. The antigen may be flea allergen FSA1, Der p1, human telomerase reverse transcriptase antigen (hTERT), prostate specific antigen (PSA), prostate specific membrane antigen (PSMA), six transmembrane epithelial antigen of the prostate (STEAP), prostate stem cell antigen (PSCA), foot and mouth disease antigen, M2, LACK, HBV, neuraminidase, hemagglutinin, or consensus thereof, a fragment thereof, or a variant thereof, for example.

The antigen may be an autologous antigen, and may induce antigen-specific iTreg cells that inhibit antigen-specific T cell function. The iTreg cells may be CD4⁺CD25⁺ and also exhibit high expression of Foxp3. The iTreg cells may be capable of specific prevention of and interference with unwanted immunity in the absence of general immunosuppression. Proliferation of the iTreg cells may be induced by high doses of interleukin 2 (IL-2). The iTreg cells may be capable of suppressing effector T cells by virtue of the presence of CD80 and CD86 ligands on activated CD4⁺ effector T cells. Once the iTreg cells are activated by a T cell receptor ligand, the presence of an antigen presenting cell may or may not be necessary in the suppression of effector T cells. After, antigenic stimulation, the iTreg cells may home to antigen-draining lymph nodes and may accumulate through cell division at the same rate as naïve T cells.

Production of the iTreg cells may require MHC Class II expression on cortical epithelial cells. The receptors may be MHC restricted, and the iTreg cells may be specific for the antigen. It may be possible via an IL-10-based mechanism to induce the iTreg cells to participate in bystander-mediated regulation.

The antigen may be associated with allergy, asthma, or an autoimmune disease. The antigen may affect a mammal, which may be a human, chimpanzee, dog, cat, horse, cow, mouse, or rat. The antigen may be contained in a protein from a mammal, which may be a human, chimpanzee, dog, cat, horse, cow, pig, sheep, mouse, or rat.

Also provided herein is a DNA that encodes the antigen. The DNA may include an encoding sequence that encodes the antigen. The DNA may also include additional sequences that encode linker or tag sequences that are linked to the antigen by a peptide bond.

Some embodiments relate to nucleic acid molecules that encode immunogenic proteins that have 95% homology to the nucleic acid coding sequences herein. Some embodiments relate to nucleic acid molecules that encode immunogenic proteins that have 96% homology to the nucleic acid coding sequences herein. Some embodiments relate to nucleic acid molecules that encode immunogenic proteins that have 97% homology to the nucleic acid coding sequences herein. Some embodiments relate to nucleic acid molecules that encode immunogenic proteins that have 98% homology to the nucleic acid coding sequences herein. Some embodiments relate to nucleic acid molecules that encode immunogenic proteins that have 99% homology to the nucleic acid coding sequences herein. In some embodiments, the nucleic acid molecules with coding sequences disclosed herein that are homologous to a coding sequence of a consensus protein disclosed herein include sequences encoding an IgE leader sequence linked to the 5′ end of the coding sequence encoding the homologous protein sequences disclosed herein.

(a) Influenza

Provided herein are antigens capable of eliciting an immune response in a mammal against one or more influenza serotypes. The antigen may be capable of eliciting an immune response in a mammal against one or more influenza serotypes, including against one or more pandemic strains, such as 209 H1N1 swine originated influenza. The antigen may be capable of eliciting an immune response in a mammal against one or more influenza serotype, including against one or more strains of swine derived human influenza. The antigen can comprise epitopes that make it particularly effective as immunogens against which anti-influenza immune response can be induced.

The antigen may be a peptide, or variant or fragment or consensus thereof, encoded by the influenza virus. The antigen may be a recombinant antigen. The antigen may be M2, neuraminidase, hemagglutinin, or a variant or consensus or fragment thereof. The neuraminidase antigen may be NP 147, which has the amino acid sequence: TYQRTRALV (SEQ ID NO:1). The neuraminidase antigen may be PR/8 IIMR-274), which is a recombinant sequence and may be purchased from Imgenex (San Diego, Calif., USA). M2 antigen may be M2e, which has the amino acid sequence: SLLTEVETPIRNEWGCRCNDSSD (SEQ ID NO:2). The influenza antigen may be from the following table.

| | SEQ |
|---|---|
| Antigenic Influenza Sequence | ID NO. |
| DNA H1 | 3 |
| Protein H1 | 4 |
| DNA H2 | 5 |
| Protein H2 | 6 |
| DNA H1 hybrid U2 | 7 |
| Protein H1 hybrid U2 | 8 |
| DNA type B hemagluttinin | 9 |
| Protein type B hemagluttinin | 10 |
| Subtype A consensus Envelope DNA sequence construct | 11 |
| Subtype A consensus Envelope protein sequence construct | 12 |
| Subtype B consensus Envelope DNA sequence construct | 13 |
| Subtype B consensus Envelope protein sequence construct | 14 |
| Subtype C consensus Envelope DNA sequence construct | 15 |
| Subtype C consensus Envelope protein sequence construct - | 16 |
| Subtype D consensus Envelope DNA sequence construct - | 17 |
| Subtype D consensus Envelope protein sequence construct - | 18 |
| Subtype B consensus Nef-Rev DNA sequence construct - | 19 |
| Subtype B consensus Nef-Rev protein sequence construct - | 20 |
| Gag consensus DNA sequence of subtype A, B, C and D | 21 |
| construct - | |
| Gag consensus protein sequence of subtype A, B, C and D | 22 |
| construct - | |
| Subtype A consensus Envelope protein sequence - | 23 |
| Subtype B consensus Envelope protein sequence | 24 |
| Subtype C consensus Envelope protein sequence - | 25 |
| Subtype D consensus Envelope protein sequence - | 26 |
| Subtype B consensus Nef-Rev protein sequence - | 27 |
| Gag consensus protein sequence of subtype A, B, C and D | 28 |
| Influenza H5N1 HA consensus sequence | 29 |
| Influenza H5N1 HA consensus sequence | 30 |
| Influenza H1N1&H5N1 NA consensus Sequence | 31 |
| Influenza H1N1&H5N1 NA consensus sequence - | 32 |
| Influenza H1N1&H5N1 M1 consensus sequence | 33 |
| Influenza H1N1&H5N1 M1 consensus sequence | 34 |
| Influenza H5N1 M2E-NP consensus sequence | 35 |
| Influenza H5N1 M2E-NP consensus sequence - | 36 |

Any of SEQ ID NOs:3-36 may comprise the IgE leader sequence: Met Asp Trp Thr Trp Ile Leu Phe Leu Val Ala Ala Ala Thr Arg Val His Ser (SEQ ID NO:37).

(b) Human Papillomavirus (HPV)

The antigen may be encoded by a human papillomavirus (HPV) sequence. The nucleic acid and/or peptide/protein sequence may be an isolated or consensus sequence. The HPV antigenic sequence may be from the following table.

| | SEQ |
|---|---|
| Antigenic HPV Sequence | ID NO |
| HPV genotype 16 E6-E7 DNA sequence - | 38 |
| HPV genotype 16 E6-E7 protein sequence - | 39 |
| HPV E6 immunodominant epitope - | 40 |
| HPV E7 immunodominant epitope | 41 |
| HPV E6 consensus sequence | 42 |
| HPV E7 consensus sequence - | 43 |

(c) Hepatitis C Virus

The antigen may be encoded by a hepatitis C virus (HCV) sequence. The nucleic acid and/or peptide/protein sequence may be an isolated or consensus sequence. The HCV antigenic sequence may be from the following table.

| | SEQ |
|---|---|
| Antigenic HCV Sequence | ID NO. |
| HCV genotype 1a and 1b consensus E1-E2 DNA sequence | 44 |
| HCV genotype 1a and 1b consensus E1-E2 protein sequence | 45 |
| HCV E1 consensus sequence | 46 |
| HCV E2 consensus sequence | 47 |

(d) FSA1

The antigen may be a peptide of the flea allergen FSA1, or a variant thereof, which may have amino acids 66-80 or amino acids 100-114 of FSA1.

(e) Der p1

The antigen may also be a peptide of Der p1, or a variant thereof. The Der p1 may have the sequence of GeneBank Access No. EU092644, the contents of which are incorporated herein by reference.

(f) Type 1 Diabetes Mellitus

The antigen may be an autoantigen involved in type 1 diabetes mellitus, or a variant thereof. The antigen may be a peptide of insulin, and may be proinsulin. The proinsulin antigen may have the sequence MALWMRLLPLLALLALWGPDPAAAFVNQHLCGSHLVEALYLVC GERGFFYTPKTRREAEDLQVGQVELGGGPGAGSLQPLALEGSLQKRGIVEQCCTSICS LYQLENYCN (SEQ ID NO:48), which may be encoded by a sequence contained in GenBank Accession No. NM_{—}000207, the contents of which are incorporated by reference herein. The antigen may be human B9-23. The insulin antigen may also have the sequence MRLLPLLALLASHLVEALYLVCGERG (SEQ ID NO:49), or LYLVCGERG (SEQ ID NO:50). The antigen may also be a insulin antigen disclosed in Wong S F, TRENDS in Molecular Medicine, 2005; 11(10), the contents of which are incorporated herein by reference. The insulin antigen may have the amino acid sequence GIVEQCCTSICSLYQ (SEQ ID NO:51).

The antigen may be a sequence of a glucose-6-phosphatase (G6P), as described in The Journal of Immunology, 2006; 176:2781-9, the contents of which are incorporated herein by reference. The G6P antigen may have the sequence of IGRP₁₃₋₂₅ (QHLQKDYRAYYTF) (SEQ ID NO:52), IGRP₂₃₋₃₅ (YTFLNFMSNVGDP) (SEQ ID NO:53), IGRP₂₂₆₋₂₃₈ (RVLNIDLLWSVPI) (SEQ ID NO:54), IGRP₂₄₇₋₂₅₉ (DWIHIDTTPFAGL) (SEQ ID NO:55), G6Pase-α₂₂₈₋₂₄₀ (KGLGVDLLWTLEK) (SEQ ID NO:56), G6Pase-α₂₄₉₋₂₆₁ (EWVHIDTTPFASL) (SEQ ID NO:57), UGRP₂₁₈₋₂₃₀ (FTLGLDLSWSISL) (SEQ ID NO:58), and UGRP₂₃₉₋₂₅₁ (EWIHVDSRPFASL) (SEQ ID NO:59).

The antigen may also be a peptide of glutamic acid decarboxylase or heat shock protein.

(g) Multiple Sclerosis

The antigen may be an autoantigen involved in multiple sclerosis (MS). The antigen may be a peptide of myelin oligodendrocyte glycoprotein (MOG), myelin basic protein (MBP), proteolipid protein (PLP), myelin-associated oligodendrocyte basic protein (MOBP), or oligodendrocyte-specific protein (OSP), or a variant thereof. The MBP antigen may be MBP66-88, MBP85-99, MBP86-105, MBP143-168, MBP83-97, or MBP85-96. The PLP antigen may be PLP30-49, PLP40-60, PLP180-199, PLP184-199, or PLP190-209. The MOG antigen may be MOG1-22, MOG34-56, or MOG64-96. The MOG antigen may also have the sequence HPIRALVGDEVELP (SEQ ID NO:60), VGWYRPPFSRVVHLYRNGKD(SEQ ID NO:61), or LKVEDPFYWVSPGVLVLLAVLPVLLL (SEQ ID NO:62). The MS antigen may also have a sequence described in Schmidt S, Mult Scler., 1999; 5(3):147-60, the contents of which are incorporated herein by reference.

(h) Autoimmune Ovarian Disease

The antigen may be an autoantigen involved in autoimmune ovarian disease. The antigen may be a peptide contained in zonapellucida (ZP) 1, 2 or 3. The ZP peptide may have the sequence of NCBI Reference Sequences NP_{—}003451.1, NP_{—}009086.4, or NP_{—}997224.2. The ZP antigen may a ZP3 peptide having the sequence ZP3 330-342 (NSSSSQFQIHGPR) (SEQ ID NO:63), ZP3 335-342 (QFQIHGPR) (SEQ ID NO:64), or ZP3 330-340 (NSSSSQFQIHG) (SEQ ID NO:65). The ZP antigen may be a peptide disclosed in Lou Y, The Journal of Immunology, 2000; 164:5251-7, the contents of which are incorporated herein by reference.

(i) Myocarditis

The antigen may be an autoantigen involved in myocarditis. The antigen may be a peptide described in Smith S C, Journal of Immunology, 1991; 147(7):2141-7, the contents of which are incorporated herein by reference. The antigen may be a peptide contained in human myosin, which may have the sequence of GeneBank Accession No. CAA86293.1. The antigen may be a peptide contained within α-myosin, and may have the sequence Ac-SLKLMATLFSTYASADTGDSGKGKGGKKKG (amino acids 614-643; where Ac is an acetyl group) (SEQ ID NO:66), GQFIDSGKAGAEKL (amino acids 735-747) (SEQ ID NO:67), or DECSELKKDIDDLE (amino acids 947-960) (SEQ ID NO:68), as disclosed in Pummerer, C L, J. Clin. Invest. 1996; 97:2057-62, the contents of which are incorporated herein by reference. The antigen may also be a Coxsackievirus B4 structural protein peptide having one of the following sequences.

TABLE 1

| Coxsackievirus B4 | Amino | |
|---|---|---|
| Structural Protein | Acids | Sequence |
| VP4 (SEQ ID NO: 69) | 1-20 | MGAQVSTQKTGAHETSLSAS |
| VP4 (SEQ ID NO: 70) | 21-40 | GNSIIHYTNINYYKDAASNS |
| VP4 (SEQ ID NO: 71) | 31-50 | NYYKDAASNSANRQDFTQDP |
| VP4 (SEQ ID NO: 72) | 41-60 | ANRQDFTQDPSKFTEPVKDV |
| VP4 (SEQ ID NO: 73) | 51-70 | SKFTEPVKDVMIKSLPALNS |
| VP2 (SEQ ID NO: 74) | 61-80 | MIKSLPALNSPTVEECGYSD |
| VP2 (SEQ ID NO: 75) | 71-90 | PTVEECGYSDRVRSITLGNS |
| VP2 (SEQ ID NO: 76) | 81-100 | RVRSITLGNSTITTQECANV |
| VP2 (SEQ ID NO: 77) | 91-110 | TITTQECANVVVGYGVWPDY |
| VP2 (SEQ ID NO: 78) | 111-130 | LSDEEATAEDQPTQPDVATC |
| VP2 (SEQ ID NO: 79) | 121-140 | QPTQPDVATCRFYTLNSVKW |
| VP2 (SEQ ID NO: 80) | 131-150 | RFYTLNSVKWEMQSAGWWWK |
| VP2 (SEQ ID NO: 81) | 151-170 | FPDALSEMGLFGQNMQYHYL |
| VP2 (SEQ ID NO: 82) | 161-180 | FGQNMQYHYLGRSGYTIHVQ |
| VP2 (SEQ ID NO: 83) | 171-190 | GRSGYTIHVQCNASKFHQGC |
| VP2 (SEQ ID NO: 84) | 181-200 | CNASKFHQGCLLVVCVPEAE |
| VP2 (SEQ ID NO: 85) | 211-230 | AYGDLCGGETAKSFEQNAAT |
| VP2 (SEQ ID NO: 86) | 221-240 | AKSFEQNAATGKTAVQTAVC |
| VP2 (SEQ ID NO: 87) | 231-250 | GKTAVQTAVCNAGMGVGVGN |
| VP2 (SEQ ID NO: 88) | 251-270 | LTIYPHQWINLRTNNSATIV |
| VP2 (SEQ ID NO: 89) | 261-280 | LRTNNSATIVMPYINSVPMD |
| VP2 (SEQ ID NO: 90) | 271-290 | MPYINSVPMDNMFRHNNFTL |
| VP2 (SEQ ID NO: 91) | 281-300 | NMFRHNNFTLMIIPFAPLDY |
| VP3 (SEQ ID NO: 92) | 321-340 | YNGLRLAGHQGLPTMLTPGS |
| VP3 (SEQ ID NO: 93) | 351-370 | SPSAMPQFDVTPEMNIPGQV |
| VP3 (SEQ ID NO: 94) | 361-380 | TPEMNIPGQVRNLMEIAEVD |
| VP3 (SEQ ID NO: 95) | 371-390 | RNLMEIAEVDSVVPINNLKA |
| VP3 (SEQ ID NO: 96) | 381-400 | SVVPINNLKANLMTMEAYRV |
| VP3 (SEQ ID NO: 97) | 391-410 | NLMTMEAYRVQVRSTDEMGG |
| VP3 (SEQ ID NO: 98) | 401-420 | QVRSTDEMGGQIFGFPLQPG |
| VP3 (SEQ ID NO: 99) | 411-430 | QIFGFPLQPGASSVLQRTLL |
| VP3 (SEQ ID NO: 100) | 421-440 | ASSVLQRTLLGEILNYYTHW |
| VP3 (SEQ ID NO: 101) | 431-450 | GEILNYYTHWSGSLKLTFVF |
| VP3 (SEQ ID NO: 102) | 441-460 | SGSLKLTFVFCGSAMATGKF |
| VP3 (SEQ ID NO: 103) | 511-530 | DDKYTASGFISCWYQTNVIV |
| VP3 (SEQ ID NO: 104) | 541-560 | MCFVSACNDFSVRMLRDTQF |
| VP1 (SEQ ID NO: 105) | 671-690 | LRRKMEMFTYIRCDMELTFV |
| VP1 (SEQ ID NO: 106) | 721-740 | VPTSVNDYVWQTSTNPSIFW |
| VP1 (SEQ ID NO: 107) | 731-750 | QTSTNPSIFWTEGNAPPRMS |
| VP1 (SEQ ID NO: 108) | 741-760 | TEGNAPPRMSIPFMSIGNAY |
| VP1 (SEQ ID NO: 109) | 751-770 | IPFMSIGNAYTMFYDGWSNF |
| VP1 (SEQ ID NO: 110) | 771-790 | SRDGIYGYNSLNNMGTIYAR |
| VP1 (SEQ ID NO: 111) | 781-800 | LNNMGTIYARHVNDSSPGGL |
| VP1 (SEQ ID NO: 112) | 791-810 | HVNDSSPGGLTSTIRIYFKP |
| VP1 (SEQ ID NO: 113) | 831-850 | SVNFDVEAVTAERASLITTG |

The antigen may be a peptide contained in a Coxsackie virus B4 structural protein as disclosed in Marttila J, Virology, 2000; 293:217-24, the contents of which are incorporated herein by reference.

The antigen may also be a peptide from group A streptococcal M5 protein. The M5 peptide may have one of the following sequences: NT4 (GLKTENEGLKTENEGLKTE) (SEQ ID NO:114), NT5 (KKEHEAENDKLKQQRDTL) (SEQ ID NO:115), B1B2 (VKDKIAKEQENKETIGTL) (SEQ ID NO:116), B2 (TIGTLKKILDETVKDKIA) (SEQ ID NO:117), B3A (IGTLKKILDETVKDKLAK) (SEQ ID NO:118), and C3 (KGLRRDLDASREAKKQ) (SEQ ID NO:119). The antigen may also be a M5 peptide from the following table.

TABLE 2

| M5 epitope | |
|---|---|
| position | Sequence |
| 27-44 | LKTKNEGLKTENEGLKTE |
| | (SEQ ID NO: 120) |
| 59-76 | KKEHEAENDKLKQQRDTL |
| (NT5) | (SEQ ID NO: 121) |
| 72-89 | QRDTLSTQKETLEREVQN |
| (NT6) | (SEQ ID NO: 122) |
| 85-102 | REVQNTQYNNETLKIKNG |
| (NT7) | (SEQ ID NO: 123) |
| 98-115 | KIKNGDLTKELNKTRQEL |
| (NT8) | (SEQ ID NO: 124) |
| 111-129 | TRQELANKQQESKENEKAL |
| (B1A) | (SEQ ID NO: 125) |
| 150-167 | TIGTLKKILDETVKDKIA |
| (B2) | (SEQ ID NO: 126) |
| 176-193 | IGTLKKILDETVKDKLAK |
| (B3A) | (SEQ ID NO: 127) |
| 1-35 | AVTRGTINDPQRAKEALDKYELENHDLKTKNEGLK |
| | (SEQ ID NO: 128) |
| 28-54 | KTKNEGLKTENEGLKTENEGLKTENEG |
| | (SEQ ID NO: 129) |
| 55-70 | LKTEKKEHEAENDKLK |
| | (SEQ ID NO: 130) |
| 103-132 | DLTKELNKTRQELANKQQESKENEKAINEL |
| | (SEQ ID NO: 131) |
| 133-162 | LEKTVKDKIAKEQENKETIGTLKKILDETV |
| | (SEQ ID NO: 132) |
| 209-223 | TIGTLKKILDETVKDK |
| | (SEQ ID NO: 133) |
| 217-237 | ISDASRKGLRRDLDASREAKK |
| | (SEQ ID NO: 134) |
| 300-319 | DASREAKKQVEKAIEEANSK |
| | (SEQ ID NO: 135) |
| 312-331 | ALEEANSKLAALEKLNKELE |
| | (SEQ ID NO: 136) |
| 329-359 | ELEESKKLTEKEKAELQAKLEAEAKQLKEQL |
| | (SEQ ID NO: 137) |
| 359-388 | AKQAEELAKLRAGKASDSQTPDTKPGNKAV |
| | (SEQ ID NO: 138) |
| 389-425 | VPGKGQAPQAGTKPNQNKAPMKETKRQLPSTGETANP |
| | (SEQ ID NO: 139) |
| 295-313 | LRRDLDASREAKKQVEKAI |
| | (SEQ ID NO: 140) |
| 305-324 | AKKQVEKALEEANSKLAALE |
| | (SEQ ID NO: 141) |
| 335-354 | KLTEKEKAELQAKLEAEAKA |
| | (SEQ ID NO: 142) |
| 345-364 | QAKLEAEAKALKEQLAKQAE |
| | (SEQ ID NO: 143) |
| 355-374 | LKEQLAKQAEELAKLRAGKA |
| | (SEQ ID NO: 144) |
| 1-25 | TVTRGTISDPQRAKEALDKYELENH |
| | (SEQ ID NO: 145) |
| 81-96 | DKLKQQRDTLSTQKETLEREVQNI |
| | (SEQ ID NO: 146) |
| 163-177 | ETIGTLKKILDETVK |
| | (SEQ ID NO: 147) |
| 1-18 | AVTRGTINDPQRAKEALD |
| | (SEQ ID NO: 148) |
| 14-31 | KEALDKYELENHDLKTKN |
| | (SEQ ID NO: 149) |
| 27-44 | LKTKNEGLKTENEGLKTE |
| | (SEQ ID NO: 150) |
| 40-58 | GLKTENEGLKTENEGLKTE |
| | (SEQ ID NO: 151) |
| 59-76 | KKEHEAENDKLKQQRDTL |
| | (SEQ ID NO: 152) |
| 72-89 | QRDTLSTQKETLEREVQN |
| | (SEQ ID NO: 153) |
| 85-102 | REVQNTQYNNETLKIKNG |
| | (SEQ ID NO: 154) |
| 98-115 | KIKNGDLTKELNKTRQEL |
| | (SEQ ID NO: 155) |
| 111-129 | TRQELANKQQESKENEKAL |
| | (SEQ ID NO: 156) |
| 124-141 | ENEKALNELLEKTVKDKI |
| | (SEQ ID NO: 157) |
| 137-154 | VKDKIAKEQENKETIGTL |
| | (SEQ ID NO: 158) |
| 150-167 | TIGTLKKILDETVKDKIA |
| | (SEQ ID NO: 159) |
| 163-180 | KDKIAKEQENKETIGTLK |
| | (SEQ ID NO: 160) |
| 176-193 | IGTLKKILDETVKDKLAK |
| | (SEQ ID NO: 161) |
| 189-206 | DKLAKEQKSKQNIGALKQ |
| | (SEQ ID NO: 162) |
| 202-219 | GALKQELAKKDEANKISD |
| | (SEQ ID NO: 163) |
| 215-232 | NKISDASRKGLRRDLDAS |
| | (SEQ ID NO: 164) |
| 228-245 | DLDASREAKKQLEAEHQK |
| | (SEQ ID NO: 165) |
| 241-258 | AEHQKLEEQNKISEASRK |
| | (SEQ ID NO: 166) |
| 254-271 | EASRKGLRRDLDASREAK |
| | (SEQ ID NO: 167) |
| 267-284 | SREAKKQLEAEQQKLEEQ |
| | (SEQ ID NO: 168) |
| 280-297 | KLEEQNKISEASRKGLRR |
| | (SEQ ID NO: 169) |
| 293-308 | KGLRRDLDASREAKKQ |
| | (SEQ ID NO: 170) |

The peptide may also be a sequence disclosed in Cunningham M W, INFECTION AND IMMUNITY, 1997; 65(9):3913-23, the contents of which are incorporated herein by reference.

(j) Rheumatoid Arthritis

The antigen may be an autoantigen involved in rheumatoid arthritis (RA). The antigen may be a peptide having the sequence Q/R, K/R, R, A, and A, described in Fox D A, Arthritis and Rheumatism, 1997; 40(4):598-609, Mackay I R, J Rheumatol, 2008; 35;731-733, or Hill J A, The Journal of Immunology, 2003; 171:538-41, the contents of which are incorporated herein by reference. The antigen may be a peptide of type II collagen, which may have the sequence of amino acids 263-270 or 184-198 of type II collagen. The type II collagen antigen may be a peptide disclosed in Staines N A, Clin. Exp. Immunol., 1996; 103:368-75 or Backlund J, PNAS, 2002; 99(15):9960-5, the contents of which are incorporated herein by reference. The type II collagen antigen may also have the sequence of amino acid residues 359-369 [C1^{III}] of type II collagen, as disclosed in Burkhardt, H, ARTHRITIS & RHEUMATISM, 2002; 46(9):2339-48, the contents of which are incorporated herein by reference.

(k) Thyroiditis

The antigen may be an autoantigen involved in thyroiditis, and may be a peptide contained in thyroid peroxidase (TPO), thyroglobulin, or Pendrin. The antigen may be described in Daw K, Springer SeminImmunopathol, 1993, 14:285-307; “Autoantigens in autoimmune thyroid diseases, The Japanese Journal of Clinical Pathology, 1989; 37(8): 868-74; Fukuma N, Clin. Exp. Immunol., 1990; 82(2):275-83; or Yoshida A, The Journal of Clinical Endocrinology & Metabolism, 2009; 94(2):442-8, the contents of which are incorporated herein by reference.

The thyroglobulin antigen may have the sequence, NIFET4QVDAQPL (SEQ ID NO:171), YSLEHSTDDT4ASFSRALENATR (SEQ ID NO:172), RALENATRDT4FIICPIIDMA (SEQ ID NO:173), LLSLQEPGSKTT4SK (SEQ ID NO:174), or EHSTDDT4ASFSRALEN (SEQ ID NO:175), where T4 is 3,5,3′,5′-tetraiodothyronine (thyroxine). The TPO antigen may have the sequence LKKRGILSPAQLLS (SEQ ID NO:176), SGVIARAAEIMETSIQ (SEQ ID NO:177), PPVREVTRHVIQVS (SEQ ID NO:178), PRQQMNGLTSFLDAS (SEQ ID NO:179), LTALHTLWLREHNRL (SEQ ID NO:180), HNRLAAALKALNAHW (SEQ ID NO:181), ARKVVGALHQIITL (SEQ ID NO:182), LPGLWLHQAFFSPWTL (SEQ ID NO:183), MNEELTERLFVLSNSST (SEQ ID NO:184), LDLASINLQRG (SEQ ID NO:185), RSVADKILDLYKHPDN (SEQ ID NO:186), or IDVWLGGLAENFLP (SEQ ID NO:187). The Pendrin antigen may have the sequence QQQHERRKQERK (SEQ ID NO:188) [amino acids 34-44 in human pendrin (GenBank AF030880)], PTKEIEIQVDWNSE (SEQ ID NO:189) [amino acids 630-643 in human pendrin], or NCBI GenBank Accession No. NP_{—}000432.1.

(l) Myasthenia Gravis

The antigen may be an autoantigen involved in myasthenia gravis (MG), and may be contained in acetylcholine receptor (AChR). The antigen may be a peptide described in Protti M A, Immunology Today, 1993; 14(7):363-8; Hawke S, Immunology Today, 1996; 17(7):307-11, the contents of which are incorporated herein by reference. The AChR antigen may be amino acids 37-429, 149-156, 138-167, 149-163, 143-156, 1-181, or 1-437 of human AChR alpha subunit.

(m) Autoimmune Uveitis

The antigen may be an autoantigen involved in autoimmune uveitis (AU), and may be contained in Human S-Antigen. The antigen may have the sequence of Peptide 19 (181-VQHAPLEMGPQPRAEATWQF-200) (SEQ ID NO:190), Peptide 35 (341-GFLGELTSSEVATEVPFRLM-356) (SEQ ID NO:191), or Peptide 36 (351-VATEVPFRLMHPQPEDPAKE-370) (SEQ ID NO:192). The antigen may be described in de Smet M D, J Autoimmun. 1993; 6(5):587-99, the contents of which are incorporated herein by reference. The antigen may also be contained in Human IRBP, and may have the sequence 521-YLLTSHRTATAAEEFAFLMQ-540 (SEQ ID NO:193). The antigen may be described in Donoso L A, J Immunol., 1989; 143(1):79-83, the contents of which are incorporated herein by reference.

(n) Other Antigens

The antigen may also be an antigen as disclosed in U.S. Patent Application Publication No. 20100143401, the contents of which are incorporated herein by reference. Other antigens may include the LACK antigen (Leishmania analogue of the receptors of activated C kinase) (36 kDa), which is highly conserved among Leishmania species and expressed by both the promastigote and amastigote forms of the parasite. Still other antigens may include human telomerase reverse transcriptase antigen (hTERT), Prostate specific antigen (PSA), prostate specific membrane antigen (PSMA), Six Transmembrane Epithelial Antigen of the Prostate (STEAP), prostate stem cell antigen (PSCA), and/or foot and mouth disease antigen.

| | SEQ |
|---|---|
| Antigenic Sequence | ID NO. |
| hTERT nucleic acid | 194 |
| hTERT amino acid sequence | 195 |
| PSA antigen 1 nucleic acid | 196 |
| PSA antigen 1 amino acid sequence | 197 |
| PSA antigen 2 nucleic acid | 198 |
| PSA antigen 2 amino acid | 199 |
| PSMA antigen 1 nucleic acid sequence | 200 |
| PSMA antigen 1 amino | 201 |
| PSMA antigen 2 nucleic acid sequence | 202 |
| PSMA antigen 2 amino acid sequence | 203 |
| STEAP antigen 1 nucleic acid sequence | 204 |
| STEAP antigen 1 amino acid sequence | 205 |
| STEAP antigen 2 nucleic acid sequence | 206 |
| STEAP antigen 2 amino acid sequence | 207 |
| PSCA antigen nucleic acid sequence | 208 |
| PSCA antigen amino acid sequence | 209 |
| Consensus VP1-VP4 subtype A and consensus C3 nucelic acid | 210 |
| sequence | |
| Consensus VP1-VP4 subtype A and consensus C3 protein | 211 |
| sequence | |
| Consensus VP1-VP4 subtype Asia 1 and consensus C3 nucelic | 212 |
| acid sequence | |
| Consensus VP1-VP4 subtype Asia 1 and consensus C3 protein | 213 |
| sequence | |
| Consensus VP1-VP4 subtype C and consensus C3 nucleic acid | 214 |
| sequence | |
| Consensus VP1-VP4 subtype C and consensus C3 protein | 215 |
| sequence | |
| Consensus VP1-VP4 subtype O and consensus C3 nucleic acid | 216 |
| sequence | |
| Consensus VP1-VP4 subtype O and consensus C3 protein | 217 |
| sequence | |
| Consensus VP1-VP4 subtype SAT 1 and consensus C3 nucleic | 218 |
| acid sequence | |
| Consensus VP1-VP4 subtype SAT 1 and consensus C3 protein | 219 |
| sequence | |
| Consensus VP1-VP4 subtype SAT2 and consensus C3 nucleic | 220 |
| acid sequence | |
| Consensus VP1-VP4 subtype SAT2 and consensus C3 protein | 221 |
| sequence | |
| Consensus VP1-VP4 subtype SAT3 and consensus C3 nucleic | 222 |
| acid sequence | |
| Consensus VP1-VP4 subtype SAT3 and consensus C3 protein | 223 |
| sequence | |
| consensus C3 nucleic acid sequence | 224 |
| consensus C3 protein sequence | 225 |
| Consensus VP1-VP4 subtype A nucleic acid sequence | 226 |
| Consensus VP1-VP4 subtype A protein sequence | 227 |
| Consensus VP1-VP4 subtype Asia 1 nucleic acid sequence | 228 |
| Consensus VP1-VP4 subtype Asia 1 protein sequence | 229 |
| Consensus VP1-VP4 subtype C nucleic acid sequence | 230 |
| Consensus VP1-VP4 subtype C protein sequence | 231 |
| Consensus VP1-VP4 subtype O nucleic acid sequence | 232 |
| Consensus VP1-VP4 subtype O protein sequence | 233 |
| Consensus VP1-VP4 subtype SAT1 | 234 |
| Consensus VP1-VP4 subtype SAT1 protein sequence | 235 |
| Consensus VP1-VP4 subtype SAT2 nucleic acid sequence | 236 |
| Consensus VP1-VP4 subtype SAT2 protein sequence | 237 |
| Consensus VP1-VP4 subtype SAT3 nucleic acid sequence | 238 |
| Consensus VP1-VP4 subtype SAT3 protein sequence | 239 |
| VP1 Asia subtype nucleic acid sequence | 240 |
| VP1 Asia subtype protein sequence | 241 |
| VP1 O subtype nucleic acid sequence | 242 |
| VP1 O subtype protein sequence | 243 |
| VP1 A subtype nucleic acid sequence | 244 |
| VP1 A subtype protein sequence | 245 |
| VP1 C subtype nucleic acid sequence | 246 |
| VP1 C subtype protein sequence | 247 |
| VP1 A subtype + VP1 C subtype nucleic acid sequence | 248 |
| VP1 A subtype + VP1 C subtype protein sequence | 249 |

(o) MHC Class II Binding Affinity

The antigen may have a high affinity for MHC Class II (MHC-II), which may increase induction of iTreg cells. The MHC-II affinity of the antigen may be an IC₅₀ of less than or equal to 50 nM. The affinity may also be an IC₅₀ of less than or equal to 100, 95, 90, 85, 80, 75, 70, 65, 60, 55, 50, 45, 40, 35, 30, 25, 20, 15, 10, 9, 8, 7, 6, 4, 3, 2, 1, 0.9, 0.8, 0.7, 0.6, 0.5, 0.4, 0.3, 0.2, or 0.1 nM.

The affinity of the antigen for MCH-II may be predicted using a computer algorithm. The algorithm may be MHCPred, as described by Guan P, Doytchinova I A, Zygouri C, Flower D R, MHCPred: bringing a quantitative dimension to the online prediction of MHC binding, Appl Bioinformatics. 2003 2:63-66; Guan P, Doytchinova I A, Zygouri C, Flower D R, MHCPred: A server for quantitative prediction of peptide-MHC binding, Nucleic Acids Res. 2003 31:3621-3624; and Hattotuwagama C K, Guan P, Doytchinova I A, Zygouri C, Flower D R, Quantitative online prediction of peptide binding to the major histocompatibility complex, J Mol Graph Model. 2004 22:195-207, the contents of which are incorporated herein by reference. The algorithm may also be NN-align or SMM-align, as described by Nielsen M and Lund O, NN-align, A neural network-based alignment algorithm for MHC class II peptide binding prediction, BMC Bioinformatics. 2009; 10:296; and Nielsen M, Lundegaard C, Lund O, Prediction of MHC class II binding affinity using SMM-align, a novel stabilization matrix alignment method, BMC Bioinformatics. 2007; 8:238, the contents of which are incorporated herein by reference.

(3) Other Components of Vaccine-Adjuvants, Excipients

The vaccine may comprise an adjuvant, which may be any nonspecific immune stimulating compound such as an interferon, and may be one or more resorcinols, or non-ionic surfactants such as polyoxyethylene oleyl ether and n-hexadecyl polyethylene ether. The adjuvant may be Freund's complete adjuvant. The adjuvant may also be IL-15, GM-CSF, LTB, mineral oil, vegetable oil, alum, aluminum compound, bentonite, silica, muramyl dipeptide derivative, thymosin, interleukin, CpG adjuvant, or MPL-TDM adjuvant (monophosphoryl Lipid A, synthetic trehalose dicorynomycolate). The vaccine may also comprise a suitable carrier, diluent, or excipient such as sterile water, physiological saline, or glucose. The vaccine may additionally be complexed with other components such as lipids, peptides, polypeptides and carbohydrates.

b. Minimally Invasive Electroporation Device

The minimally invasive electroporation device (“MID”) may be an apparatus for injecting the linear expression cassette (“LEC”) described above and associated fluid into body tissue. The device may comprise a hollow needle; and LEC and fluid delivery means, wherein the device is adapted to actuate the fluid delivery means in use so as to concurrently (preferably automatically) inject LEC into body tissue during insertion of the needle into the said body tissue. This has the advantage that the ability to inject the LEC and associated fluid gradually while the needle is being inserted leads to a more even distribution of the fluid through the body tissue. The pain experienced during injection may be reduced due to the distribution of the LEC being injected over a larger area.

The MID may inject LEC into tissue without the use of a needle. The MID may inject the LEC as a small stream or jet with such force that the LEC pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. No. 6,520,950; U.S. Pat. No. 7,171,264; U.S. Pat. No. 6,208,893; U.S. Pat. No. 6,009,347; U.S. Pat. No. 6,120,493; U.S. Pat. No. 7,245,963; U.S. Pat. No. 7,328,064; and U.S. Pat. No. 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired LEC in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the LEC into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver LECs to all types of tissues, particularly to skin and mucosa. In some embodiments, a needle-free injector may be used to propel a liquid that contains the LEC the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the LEC. By pulsing between multiple pairs of electrodes in a multiple electrode array, preferably set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle DNA injectors that deliver the LEC and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA® device and system (Inovio Pharmaceuticals, Inc., Blue Bell, Pa.), which is a a modular electrode system, that facilitates the introduction of a macromolecule, such as a LEC, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

(1) Elgen 1000 System

The MID may be an Elgen 1000 system (Inovio Pharmaceuticals, Inc., Blue Bell, Pa.). The Elgen 1000 system may comprise device that provides a hollow needle; and fluid delivery means, wherein the apparatus is adapted to actuate the fluid delivery means in use so as to concurrently (preferably automatically) inject fluid into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumour tissue, skin or liver tissue but will preferably be muscle tissue.

Preferably the apparatus further comprises needle insertion means for guiding insertion of the needle into the body tissue The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus preferably includes means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. Preferably therefore, the fluid delivery means comprise piston driving means adapted to inject fluid at a controlled rate. The piston driving means could for example be activated by a servo motor. Preferably however, the piston driving means are actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it preferably further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so user's have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### 3. Kit

Provided herein is a kit, which may be used for vaccinating a subject. The kit may comprise an LEC and a MID. The kit can further comprise instructions for using the kit and conducting the analysis, monitoring, or treatment, including prophylactic vaccination.

The kit may also comprise one or more containers, such as vials or bottles, with each container containing a separate reagent. The kit may further comprise written instructions, which may describe how to perform or interpret an analysis, monitoring, treatment, or method described herein.

The present invention has multiple aspects, illustrated by the following non-limiting examples.

### EXAMPLES

### Example 1

### Materials and Methods

The following materials and methods were used in Example 1. Plasmids and LEC's. The backbones of pNP and pM2 are pMB76.5. pNP encodes NP of influenza A/Puerto Rico/34 and pM2 encodes M2 of New Caledonia/99. The plasmids were constructed by inserting NP or M2 gene produced by GENEART into the multiple cloning site of pMB76.5. The linear expression cassette lecNP or lecM2 contains CMV promoter, intron for splicing, full length gene of NP or M2 with stop codon and polyadenylation signal. Plasmids were prepared in house using QIAGEN endotoxin free plasmid kits. The LECs used in this research were manufactured by Vandalia, Va. PCR amplification products were purified from unincorporated dNTPs and primers using membrane filtration and ethanol precipitation. The integrity of the PCR products was assessed using agarose gel electrophoresis and Agilent Bioanalyzer microfluidic chips. The quantity of DNA were confirmed via PicoGreen Fluorimetry and using a Nanodrop UV spectrophotometer. The sequence of the bulk linear PCR products was confirmed by DNA sequencing.

Synthetic peptides and recombinant protein. NP147 (TYQRTRALV) (SEQ ID NO:1) and peptides corresponding to the M2e (SLLTEVETPIRNEWGCRCNDSSD) (SEQ ID NO:2) of influenza were synthesized by Invitrogen. The recombinant NP (rNP) corresponding to the NP amino acid sequence of PR/8 (IMR-274) were purchased from Imgenex (San Diego, Calif., USA).

In vitro antigen expression. Appropriate expression of the antigens by both plasmid and LEC forms were confirmed by in vitro transfection of HEK 293 cells with TransIT-293 Transfection Reagent (Mirus, Madison, Wis.). HEK 293 cells were grown on regular tissue culture plates for flow cytometry and on chamber slides (Nalge Nunc International) for immunofluorescence microscopy. Transfection was done according to manufacture's protocol. Expression of antigens was detected by surface and intracellular staining (CytoFix/CytoPerm kit BD Biosciences) with antigen specific monoclonal antibodies first. To stain for NP, anti Influenza anti-influenza A group-FITC (clone IA52, Argene Inc. North Massapequa—N.Y.) were used. To stain M2e, anti-Influenza A M2 (clone 14C2, Abcam, Cambridge, Mass.) and then anti-mouse IgG (Fab)-FITC (Sigma-Aldrich, Saint Louis, Mo.) were used. Stained cells were then analyzed by flow cytometry or immunofluorescence microscopy.

Minimally Invasive Device. Electrode arrays consisting of a 4×4 gold plated trocar needle of 0.0175 inch diameter at a 1.5 mm spacing were constructed to be used in conjunction with the ELGEN1000 (Inovio Pharm., San Diego) pulse generator.

Mice and immunizations. All immunizations were conducted at Bioquant (San Diego, Calif., USA) according ethical guidelines that had been approved by the ethical committee of Bioquant. Female Balb C mice (6-10 weeks old) were purchased from Harlan Teklad and were shaved prior to treatment.

Mice were injected intradermally (Mantoux method (needle parallel to skin)—29 gauge Insulin needle) with 50 μl of 1× PBS containing the desired dose of plasmid or equal mole of each LEC. Mice were injected intramuscularly into the quadriceps with 50 μl of 1× PBS containing the desired dose of plasmid.

Dermal Device Electroporation—Immediately following injection of DNA, the MID-II dermal device was applied to the site of dermal injection. The array was “wiggled” at the injection site to ensure good contact and electrotransfer achieved through pulse generation from the ELGEN 1000. The parameters used were three 15 volt pulses of 100 ms duration.

ELISA for detection of anti-NP and anti-M2e. Antibody responses against NP and M2 were evaluated by ELISA using serum from immunized mice. Mice were bled retro-orbital two weeks after last immunization. M2e peptides (15 μg/mL) or rNP (5 μg/mL) were coated to the plate by filling the microwells of a Nunc Maxi-Sorp Immuno Plate with 50 uL of the diluted antigen. The plates were incubated at 4° C. overnight. Unbound antigens were washed off the plate by an automatic plate wash using PBS with 0.05% Tween-20. The plates were blocked for non-specific binding by adding 200 uL of PBS with 0.5% BSA for one hour at 37° C. After washing as above, serum was diluted 1:50 in PBS with 0.2% BAA and 0.005% Tween-20 and added to the first well. A serial dilution was done by diluting 1:5 for every well. The serum was incubated for two hours at 37° C. before washing. Anti-mouse IgG-biotin (B9904-5 ml; Sigma-Aldrich, St Louis, Mo., USA) was diluted 1:10000 and 50 μL is added to each well and incubated one hour at 37° C. before washing. This was followed by adding 50 μL streptavidin-HRP (Southern Biotech, Birmingham, Ala., USA) diluted 1:1000 to each well and incubated one hour at 37° C. before washing. The final step was done by adding 50 μL HRP substrate (P-9187, Sigma-Aldrich) and incubating at room temperature in the dark for 10 minutes before reading the optical density (OD) at 450 nm. A reading was considered positive if the OD was three times higher then the OD from naïve mice serum. Results were presented as end-point titer, i.e. the last dilution where the OD was more or equal to three times higher then the naïve serum.

Intracellular cytokine staining and flow cytometry analysis. CTL responses against NP147 (TYQRTRALV) (SEQ ID NO:1) and M2e (MSLLTEVETPIRNEWGCRCNDSSD) (SEQ ID NO:2) were studied by intracellular cytokine staining (ICS).

Single cell suspension of mouse spleen was used for ICS and flow cytometry analysis. In each well of a 24-well plate, 10 million of splenocytes were stimulated in 0.5 ml T cell culture media (RPMI medium with 10% FBS, 2 mM L-glutamine, 0.1 mM MEM non-essential amino acid, 1 mM MEM sodium pyruvate, 0.05 mM 2-ME, 100 U/ml penicillin, and 100 μg/ml streptomycin) containing GolgiStop (4 μl/6 ml), and stimulating peptides (each peptide: 10 μg/ml) at 37° C. for 8 hr. Irrelevant peptide were used for negative control. PMA and ionomycin (PMA: 10 ng/ml, ionomycin: 500 ng/ml) were used for positive control.

1-5 million stimulated splenocytes were transferred into the wells of U-bottom 96-well plate. Red blood cells were lysed and then cells were washed three times. Cells were then surface stained with anti-CD3-PerCP, anti-CD4-PE and anti-CD8-APC on ice in the dark for 15 min and then washed twice. IFNγ production was measured by ICS using anti-IFNγ-FITC and Cytofix/Cytoperm kits following manufacture's protocol. All monoclonal antibodies (BD Pharmingen, San Diego, Calif.) for ICS were used at 0.5 μg/ml.

Net numbers of IFNγ positive CD8 cells per million splenocytes were calculated by subtracting the negative peptide control for each group of mice.

Influenza virus challenge. Balb/c mice in groups of 10 were immunized by MID-II on week 0, 3, and 6. 10 μg of each plasmid and equal mole of each LEC were used per mouse for each immunization. On week 8, blood samples were taken from each mouse to study antibody responses against our antigens. Equal amount of blood from each mouse were pooled for each group to study T cell responses against flu. On week 10, Flu challenge experiments were conducted using BSL IV protocols at The National Microbiology Laboratory, Public Health Agency of Canada, Winnipeg, MB, Canada according to ethical guidelines that have been approved by their ethical committee. 5×10⁵ TCID/Mouse of H1N1 influenza strain A/Canada/AB/RV1532/2009 or 100×LD50 H5N1 strain A/Vietnam/1203/04 were used for each mouse. Mice were monitored for survival and body-weight every day for 21 days after challenge.

### Example 2

### LEC Synthesis and In Vitro Expression

pNP, a plasmid encoding NP of influenza A/Puerto Rico/34 was used to synthesize the corresponding LEC lecNP using PCR based technology (Vandalia). And pM2 a plasmid encoding M2 of influenza A/New Caledonia/99 was used to synthesize the corresponding LEC perM2. As shown in FIG. 1, the linear expression cassette contains elements essential for expression in mammalian cells: CMV promoter, intron, and gene of interest followed by SV40 polyadenylation signal.

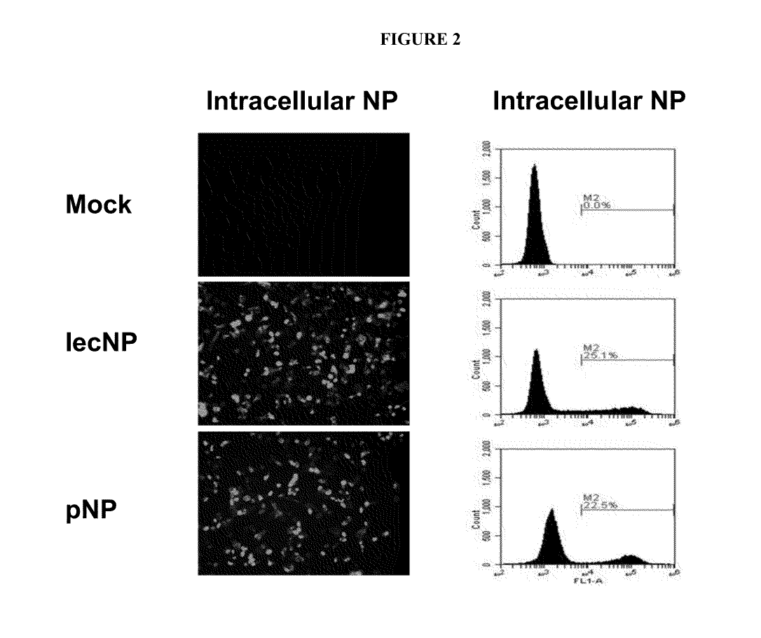

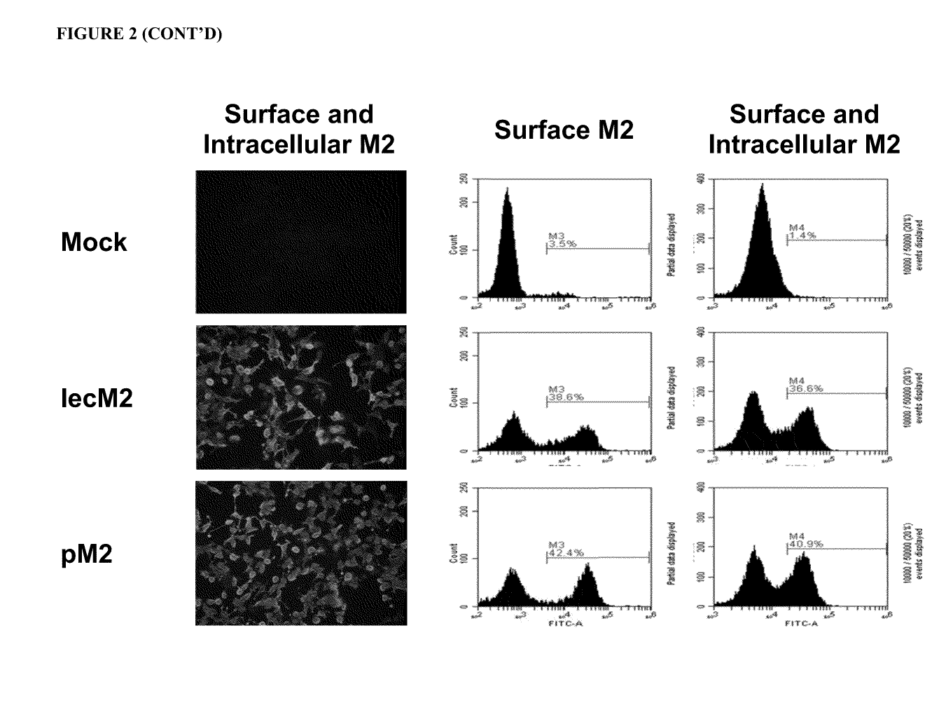

In vitro expression of the influenza antigens using LEC encoding NP or M2 was studied. HK 293 cells were transfected with LEC encoding NP and compared to expression from plasmid DNA expressing NP. In vitro expression experiments have strict DNA and lipid dose requirements, therefore equal weights of DNA were used in this study. Twenty-four hours after transfection, cells were fixed and permeablized and expression of intracellular NP was observed by immunofluorescence microscopy. As shown in FIG. 2A, appropriate expression of NP antigen using pNP and lecNP were observed by immunofluorescence microscopy after fixing/permeabilizing the transfected cells and staining with FITC conjugated anti-NP. The fraction of cells expressing NP was analyzed by flow cytometry analysis following transfection with plasmids or LEC constructs and intracellular staining of NP antigen. 25% of cells were transfected by the lecNP, very similar to 22.5% of tranfection of cells achieved by pNP.

Intracellular expression of M2 in HK 293 cells was also achieved following transfection of cells using lecM2. Intracellular staining of M2 antigen was carried out before immunofluorescence microscopy and flow cytometry analysis. No significant difference in terms of fraction of antigen positive cells (about 40%) and mean fluorescence intensity was observed between the plasmid and LEC forms of the M2 DNA vaccine. Correct surface expression of M2 following transfection by LEC is also confirmed by antibody surface staining without fixation and permeabilization. In corroboration of the ICS data, flow cytometry analysis showed no significant difference in terms of fraction of antigen positive cells (about 40%) and mean fluorescence intensity between the lecM2 and pM2. Therefore, these results indicate appropriate expression of NP and M2 antigens following transfection by the LEC forms of the DNA vaccines in HK293 cells.

### Example 3

### Sustained Antibody Response and Protective Immunity

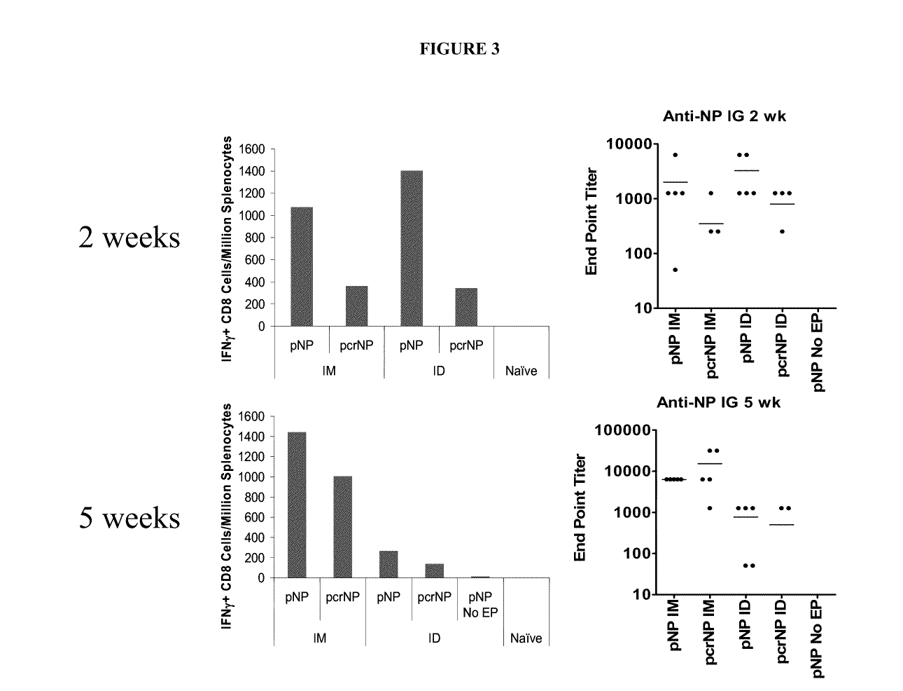

Having established appropriate expression of the LEC forms of the influenza antigens in cells in culture, we were curious to investigate the immune response induced following in vivo expression of these constructs in mouse model. Balb/c mice (5 per group) were immunized once with 10 ug plasmid pNP or equal mole of LEC lecNP (5.1 ug) delivered via intradermal electroporation (ID EP) using MID-II or ID delivery with no electroporation. Two weeks following the immunization, serum samples were taken to evaluate antibody responses and splenocytes were used to evaluate cellular responses against NP. As shown in FIG. 3, following in vivo plasmid transfection, strong antibody and cellular immune responses were induced via ID EP. Equal molar LEC delivery followed by ID EP also resulted in detectible antibody and cellular immune responses but responses were weaker compared to the responses induced by plasmid. For both antibody and T cell responses, plasmid induced approximately 4 fold higher responses than LEC. Mice who received plasmid vaccination without ID EP did not elicit significant antibody or cellular responses.

Five weeks following the initial immunization, both humoral and cellular responses were detectible in the LEC ID EP group and plasmid ID EP group. Although there was a significant difference in titers between the LEC and plasmid groups at two weeks post vaccination, the titers were more comparable at week 5 although the plasmid immunizations continued to elicit stronger responses. For antibody responses, plasmid was 1.5 fold higher than LEC. For T cell response, plasmid was 2 fold higher than LEC. Although the plasmid responses were higher at both time points, the LEC responses appeared more sustained over time. Again, immune responses in the group which received plasmid without EP were very low at week five.

In the previous experiment, we observed that mice immunized with the LEC DNA vaccine induced immune responses that might decrease at a slower rate compared to responses induced by plasmid immunization. This more sustained response could be due to low-anti vector immunity, making this platform ideal for prime/boost vaccination strategies.

In this experiment, we studied the immunogenicity of LEC in a DNA prime DNA boost setting. Balb/c mice (10 per group) were immunized with 10 μg pNP plus 30 μg pM2 DNA or equal mole of lecNP (5.1 ug) and lecM2 (11.3 ug). Both DNA vaccines were delivered via the MID-II ID EP delivery system or ID delivery without EP.

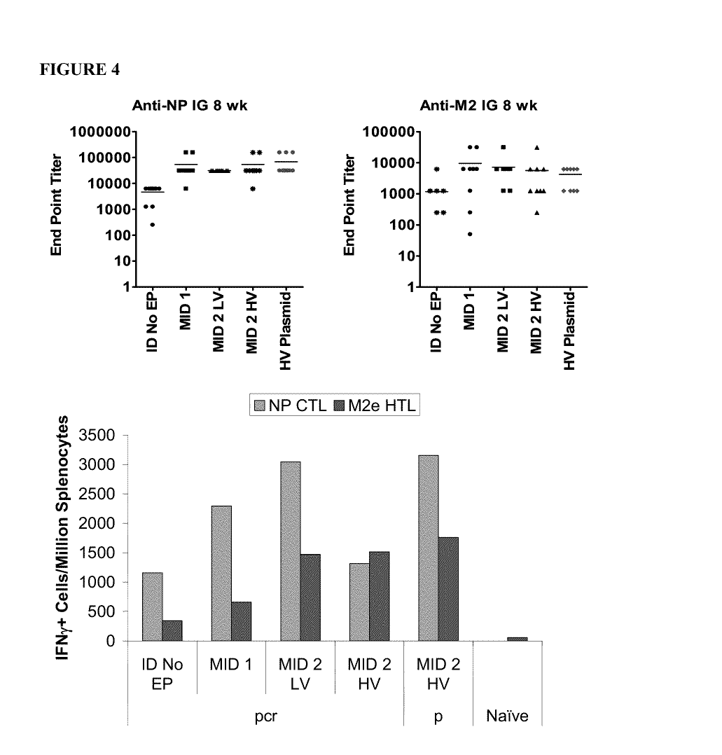

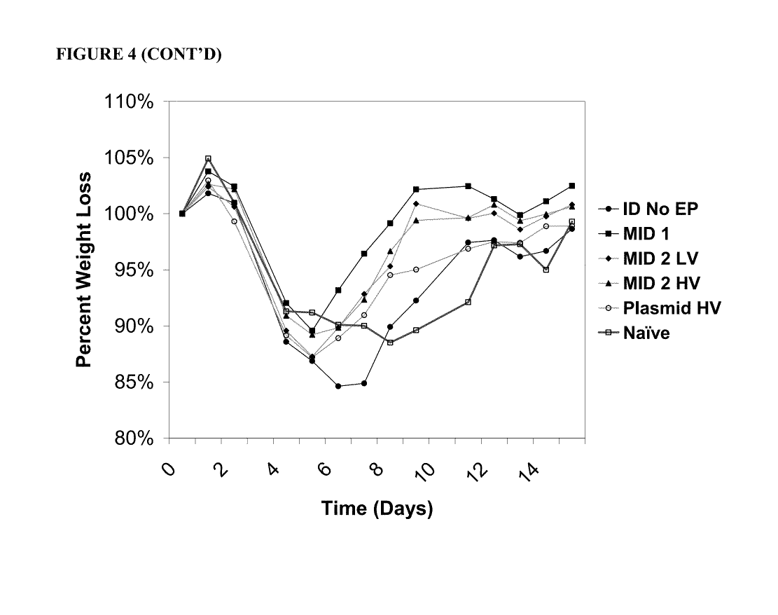

Mice were primed at week zero and boosted at week three and week six. Mice were bled two weeks after the last boost and antibody responses against NP and M2 were measured by ELISA. As shown in FIG. 4A, strong and consistent levels of antibody responses were induced by LECs delivered via MID-II ID EP. The antibody titers are significantly higher than ID delivery of LECs without EP. Most importantly, when MID-II ID EP was used to deliver DNA, LEC induced similar levels of antibody responses compared to plasmid. T cell responses were measured by intracellular cytokine staining of IFNγ using pooled blood for each group. CTL responses against NP 147 and HTL response M2e are shown in FIGS. 4B & 4C. The results indicated T cell responses can be induced by LEC. The responses are further enhanced by EP when LECs were delivered ID EP. LEC induced similar immune responses compared to equal mole of plasmid after multiple immunizations. In a bid to establish if these responses were protective, the mice were then challenged with 5×10⁵ TCID/Mouse of the current human/swine Flu strain A/Canada/AB/RV1532/2009 10 weeks after the last immunization. Since this strain is non lethal in a mouse model, only the body weights of the mice were monitored as a readout of morbidity. As shown in FIG. 4D, although all groups of mice lost weight initially, mice immunized with LECs combined with MID-II ID EP recovered faster than naïve or mice immunized ID without EP and the elicited protection correlated with immune response data well.

### Example 4

### Electroporation Delivery of LEC

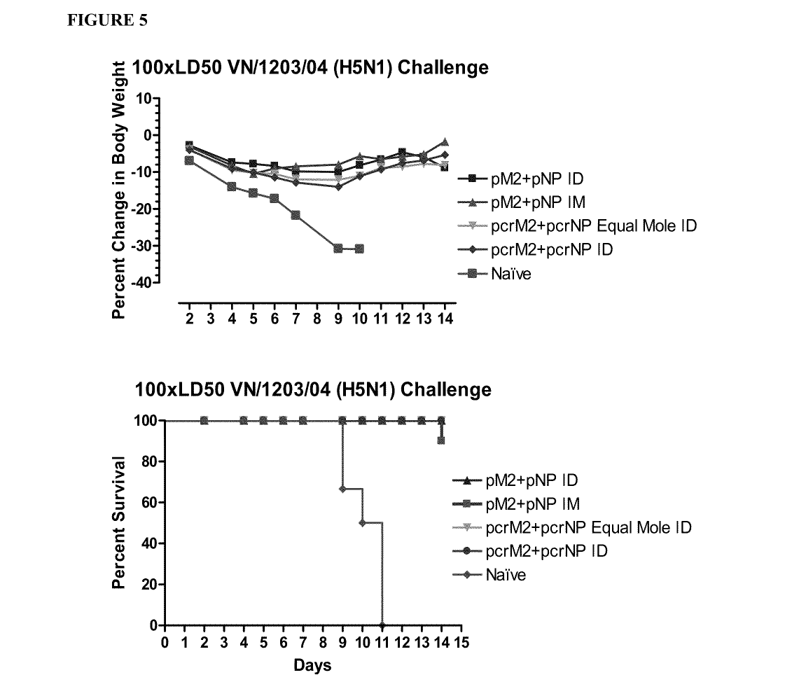

In a further study, Balb/c mice were immunized with 30 μg pNP plus 30 μg pM2 or equal moles of lecNP plus lecM2 DNA via the MID-II ID EP device. The mice were boosted three weeks later. Ten weeks after the first boost the animals were boosted again with 100 μg pNP plus 100 μg pM2 or equal moles of lecNP plus lecM2 DNA via ID EP. On week fifteen, animals were challenged with 100×LD50 H5N1 strain A/Vietnam/1203/04. As expected, all naïve mice died. However, the immunized mice demonstrated 100% survival rates following the lethal challenge as shown in FIG. 5B. Importantly, mice showed only very minor weight loss as shown in FIG. 5A suggesting that the vaccine platform also reduced morbidity.

We claim:

1. A nucleic acid construct capable of expressing a desired antigen in cells of a subject, comprising:
a linear expression cassette comprising an antigen encoding sequence selected from the group consisting of SEQ ID NOs: 3, 5, 7, 9, 11, 13, 15, 17, 19, 21, 23, 25, 27, 29, 31, 33, 35, 38, 44, 46, 194, 196, 198, 200, 202, 204, 206, 208, 210, 212, 214, 216, 218, 220, 222, 224, 226, 228, 230, 232, 234, 236, 238, 240, 242, 244, 246, 248, and sequences that are at least 98% similar thereof; or antigen encoding sequence encoding an antigen selected from the group consisting of SEQ ID NOs: 1, 2, 4, 6, 8, 10, 12, 14, 16, 18, 20, 22, 24, 26, 28, 30, 32, 34, 36, 39-43, 45, 47, 48-193, 195, 197, 199, 201, 203, 205, 207, 209, 211, 213, 215, 217, 219, 221, 223, 225, 227, 229, 231, 233, 235, 237, 239, 241, 243, 245, 247, and 249, and sequences that are at least 98% similar thereof.

2. The linear expression cassette of claim 1, wherein the antigen encoding sequence is SEQ ID NO:1 or SEQ ID NO:2.

3. A non-invasive method of vaccination comprising electroporating a linear expression cassette into a subject in need thereof, wherein the linear expression cassette comprises a nucleic acid encoding one or more antigens.

4. The method of claim 3, wherein the electroporation route is selected from the group consisting of intradermal and intramuscular.

5. The method of claim 3, wherein the antigen is selected from the group consisting of M2, LACK, HBV, neuraminidase, hemagglutinin, a variant thereof, and a consensus thereof.

6. The method of claim 3, wherein the linear expression cassette further comprises a promoter selected from the group consisting of CMV, SV40 early promoter, SV40 later promoter, metallothionein promoter, murine mammary tumor virus promoter, Rous sarcoma virus promoter, and polyhedrin promoter.

7. The method of claim 3, wherein the linear expression cassette is electroporated with a minimally-invasive electroporation device.

8. The method of claim 3, wherein the linear expression cassette is selected from the group consisting of perNP and perM2.

9. A vaccination kit comprising an electroporation device and a linear expression cassette, wherein the linear expression cassette comprises a nucleic acid encoding one or more antigens.

10. The kit of claim 9, wherein the one or more antigens is selected from the group consisting of M2, LACK, HBV, neuraminidase, hemagglutinin, a variant thereof, and a consensus thereof.

11. The kit of claim 9, wherein the electroporation device is a minimally-invasive electroporation device.

12. The kit of claim 9, wherein the one or more linear expression cassettes is selected from the group consisting of perNP and perM2.


SEQUENCE LISTING (249 sequences)

SEQ ID NO: 1 - protein, 9 aa, Artificial Sequence
  neuraminidase antigen  NP147
TYQRTRALV

SEQ ID NO: 2 - protein, 23 aa, Artificial Sequence
  M2 antigen M2e
SLLTEVETPIRNEWGCRCNDSSD

SEQ ID NO: 3 - DNA, 1695 bp, Artificial Sequence
  DNA H1
atgaaggctatcctcgtcgtgctgctgtacaccttcgccaccgccaacgccgataccctgtgcatcggctaccacgccaacaacagcaccgacaccgtggataccgtgctggaaaagaacgtgaccgtgacccacagcgtgaacctgctggaagataagcacaacggcaagctgtgcaagctgagaggcgtggcccctctgcacctgggcaagtgcaatatcgccggctggattctgggcaaccccgagtgcgagagcctgtctaccgctagctcctggtcctacatcgtggagacaagcagcagcgacaacggcacctgttaccccggcgacttcatcgactacgaggaactgcgggagcagctgagcagcgtgtccagcttcgagcggttcgagatcttccccaagaccagctcctggcccaaccacgacagcaacaagggcgtgaccgccgcctgtcctcacgctggcgccaagagcttctacaagaacctgatctggctggtcaagaagggcaacagctaccccaagctgagcaagagctacatcaacgacaagggcaaagaggtcctcgtcctctggggcatccaccaccctagcaccagcgccgaccagcagagcctgtaccagaacgccgacgcctacgtgttcgtgggctcatctcggtacagcaagaagttcaagcccgagatcgccatcagacccaaagtgcgggaccaggaaggccggatgaactactactggaccctggtggagcccggcgacaagatcaccttcgaggccaccggcaatctggtggtgcccagatacgccttcgccatggaaagaaacgccggcagcggcatcatcatcagcgacacccccgtgcacgactgcaacaccacctgtcagacccccaagggcgccatcaacaccagcctgcccttccagaacatccaccccatcaccatcggcaagtgccctaagtacgtgaagtccactaagctcagactggccaccggcctgagaaacgtgcccagcatccagagcagaggcctgtttggcgccattgccggctttatcgagggcggctggaccggaatggtggacgggtggtacggctaccaccaccagaatgagcagggcagcggctacgccgccgacctgaagtccacacagaacgccatcgacgagatcaccaacaaagtgaacagcgtgatcgagaagatgaacacccagttcaccgccgtgggcaaagagttcaaccacctggaaaagcggatcgagaacctgaacaagaaggtggacgacggcttcctggacatctggacctacaacgccgagctgctggtgctgctggaaaacgagcggaccctggactaccacgactccaacgtgaagaatctgtacgagaaagtgcggagccagctgaagaacaacgccaaagagatcggcaacggctgcttcgagttctaccacaagtgcgacaacacctgtatggaaagcgtgaagaacggcacctacgactaccccaagtacagcgaggaagccaagctgaaccgggaagagatcgacggcgtgaagctggaaagcacccggatctaccagatcctggccatctactctactgtggccagctcactggtgctggtggtgtccctgggcgccatctccttttggatgtgctccaacggcagcctgcagtgccggatctgc

SEQ ID NO: 4 - protein, 566 aa, Artificial Sequence
  protein H1
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 5 - DNA, 1719 bp, Artificial Sequence
  DNA H2
ggtaccaagcttgccaccatggccatcatctacctgatcctgctgttcaccgccgtgcggggcgaccagatctgcatcggctaccacgccaacaacagcaccgagaaggtggacaccatcctggaacggaacgtgaccgtgacccacgccaaggacatcctggaaaagacccacaacggcaagctgtgcaagctgaacggcatcccccccctggaactgggcgactgcagcattgccggctggctgctgggcaaccccgagtgcgaccggctgctgtccgtgcccgagtggagctacatcatggaaaaagagaacccccgggacggcctgtgctaccccggcagcttcaacgactacgaggaactgaagcacctgctgtccagcgtgaagcacttcgagaaggtgaaaatcctgcccaaggaccggtggacccagcacaccaccaccggcggcagcagagcctgtgccgtgagcggcaaccccagcttcttccggaacatggtgtggctgaccaagaagggcagcaactaccccgtggccaagggcagctacaacaacacctccggagaacagatgctgatcatctggggcgtgcaccaccccaacgacgagacagagcagcggaccctgtaccagaacgtgggcacctacgtgagcgtgggcaccagcaccctgaacaagcggagcacccccgagatcgccacccggcccaaggtgaacggcctgggcagccggatggaattcagctggaccctgctggacatgtgggacaccatcaacttcgagagcaccggcaacctgatcgcccccgagtacggcttcaagatcagcaagcggggcagcagcggcatcatgaaaaccgagggcaccctggaaaactgcgagacaaagtgccagacccccctgggcgccatcaacaccaccctgcccttccacaacgtgcaccccctgaccatcggcgagtgccccaagtacgtgaagagcgagaagctggtgctggccaccggcctgcggaacgtgccccagatcgagagcaggggcctgttcggcgccattgccggattcatcgagggcggctggcagggcatggtggacgggtggtacggctaccaccacagcaacgaccagggcagcggctacgccgccgacaaagagagcacccagaaggccttcgacggcatcaccaacaaggtgaacagcgtgatcgagaagatgaacacccagttcgaggccgtgggcaaagagttcagcaacctggaacggcggctggaaaacctgaacaagaaaatggaagatggcttcctggacgtgtggacctacaacgccgagctgctggtgctgatggaaaacgagaggaccctggacttccacgacagcaacgtgaagaacctgtacgacaaagtgcggatgcagctgcgggacaacgtgaaagagctgggcaacggctgcttcgagttctaccacaagtgcgacgacgagtgcatgaactccgtgaagaacggcacctacgactaccctaagtacgaggaagagtccaagctgaaccggaacgagatcaagggcgtgaagctgtccagcatgggcgtgtaccagatcctggccatctacgccaccgtggccggcagcctgagcctggctattatgatggctggcatcagcttttggatgtgcagcaacggcagcctgcagtgccggatctgcatctgatgactcgagctc

SEQ ID NO: 6 - protein, 562 aa, Artificial Sequence
  protein H2
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 7 - DNA, 1695 bp, Artificial Sequence
  DNA H1 hybrid U2
aaggccaagctgctggtgctgctgtgcaccttcgccgccaccaacgccgacaccatctgcatcggctaccacgccaacaacagcaccgacaccgtggataccgtgctggaaaagaacgtgaccgtgacccacagcgtgaacctgctggaagataagcacaacggcaagctgtgcaagctgaagggaatcgcccccctgcagctgggcaagtgcaatatcgccggctggattctgggcaaccccgagtgcgagagcctgagcagcaagagcagctggtcctacatcgtggaaacccccaacagcgagaacggcacctgttaccccggcgacttcgccgactacgaggaactgcgcgagcagctgagcagcgtgtccagcttcgagagattcgagatcttccccaagaccagcagctggcccaaccacgacgtgaccaagggcgtgaccgctagctgtagccacgcaggcgccagcagcttctacaagaacctgctgtggctgaccaagaagaacggcagctaccccaagctgagcaagagctacatcaacaacaaagaaaaagaggtgctggtcctctggggcgtccaccaccccagcacaatcgccgaccagcagagcctgtaccagaacgagaacgcctacgtgtccgtgggcagcagccactacagccggaagttcacccccgagatcgccaagcggcccaaagtgcgggaccaggaaggccggatcaactactactggaccctgctggaacccggcgacaccatcatcttcgaggccaacggcaacctgatcgcccccagatacgccttcgccctgagcagaggcttcggcagcggcatcatcatcagcaacgcccccatgcacgactgcgacaccaagtgccagacccctcagggcgccatcaacagcagcctgcccttccagaacatccaccccgtgaccatcggcgagtgccccaaatacgtgcggagcaccaagctgcggatggccaccggcctgcggaacatccccagcatccagagcagaggcctgttcggcgccattgccggcttcatcgagggcggctggaccggaatggtggacgggtggtacggctaccaccaccagaatgagcagggcagcggctacgccgccgaccagaagtccacccagaacgccatcgacggcatcaccaacaaagtgaacagcgtgatcgagaagatgaacacccagttcaccgccgtgggcaaagagttcaacaagctggaaaagcggatggaaaacctgaacaagaaggtggacgacggcttcctggacatctggacctacaacgccgaactgctcgtgctgctggaaaacgagcggaccctggacttccacgacagcaacgtgaagaacctgtacgagaaagtgaagtcccagctgaagaacaacgccaaagagatcggcaacggctgcttcgagttctaccacaagtgcaacaacgagtgcatggaaagcgtgaagaacggaacctacgactaccccaagtacagcgaggaaagcaagctgaaccgggaagagatcgacggcgtgaagctggaatccatgggcgtgtaccagatcctggccatctacagcaccgtggctagcagcctggtgctgctggtgtccctgggcgccatctccttttggatgtgctccaacggcagcctgcagtgccggatctgcatc

SEQ ID NO: 8 - protein, 565 aa, Artificial Sequence
  Protein H1 hybrid U2
KAKLLVLLCTFAATNADTICIGYHANNSTDTVDTVLEKNVTVTHSVNLLEDKHNGKLCKLKGIAPLQLGKCNIAGWILGNPECESLSSKSSWSYIVETPNSENGTCYPGDFADYEELREQLSSVSSFERFEIFPKTSSWPNHDVTKGVTASCSHAGASSFYKNLLWLTKKNGSYPKLSKSYINNKEKEVLVLWGVHHPSTIADQQSLYQNENAYVSVGSSHYSRKFTPEIAKRPKVRDQEGRINYYWTLLEPGDTIIFEANGNLIAPRYAFALSRGFGSGIIISNAPMHDCDTKCQTPQGAINSSLPFQNIHPVTIGECPKYVRSTKLRMATGLRNIPSIQSRGLFGAIAGFIEGGWTGMVDGWYGYHHQNEQGSGYAADQKSTQNAIDGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREEIDGVKLESMGVYQILAIYSTVASSLVLLVSLGAISFWMCSNGSLQCRICI

SEQ ID NO: 9 - DNA, 1749 bp, Artificial Sequence
  DNA type B hemagluttinin
aaggccatcatcgtgctgctgatggtggtcacaagcaacgccgaccggatctgcaccggcatcaccagcagcaacagcccccacgtggtcaaaaccgccacccagggcgaagtgaacgtgaccggcgtgatccccctgaccaccacccccaccaagagccacttcgccaacctgaagggcaccaagacccggggaaagctgtgccccaagtgcctgaactgcaccgacctggacgtggccctgggcagacctatgtgcgtgggcaccacccctagcgccaaggccagcatcctgcacgaagtgcggcccgtgaccagcggctgcttccccatcatgcacgaccggaccaagatccggcagctccccaacctgctgcggggctacgagaacatccggctgagcacccagaacgtgatcaacgccgagaaggcccctggcggcccttacagactgggcacaagcggctcttgccccaacgccaccagcaagagcggctttttcgccacaatggcctgggccgtgcccaaggacaacaacaagaccgccaccaaccccctgaccgtggaagtgccctacatctgcaccgagggcgaggaccagatcaccgtgtggggcttccacagcgataacaagacccagatgaagaacctgtacggcgacagcaacccccagaagttcaccagctccgccaacggcgtgaccacccactacgtgtcccagatcggcggcttccccgaccagacagaggatggcggcctgccccagagcggcagaatcgtggtggactacatggtgcagaagcccggcaagaccggcaccatcgtgtaccagcggggcatcctgctgccccagaaagtgtggtgcgccagcggccggtccaaagtgatcaagggcagcctgcctctgatcggcgaggccgattgcctgcacgagaagtacggcggcctgaacaagagcaagccctactacaccggcgagcacgccaaagccatcggcaactgccccatctgggtcaaaacccccctgaagctggccaacggcaccaagtaccggcctcccgccaagctgctgaaagagcggggcttcttcggcgctatcgccggctttctggaaggcggctgggagggcatgatcgccggctggcacggctacacatctcacggcgctcatggcgtggccgtggccgctgatctgaagtccacccaggaagccatcaacaagatcaccaagaacctgaacagcctgagcgagctggaagtgaagaatctgcagcggctgagcggcgccatggacgagctgcacaacgagatcctggaactggacgagaaggtggacgacctgcgggccgacaccatctccagccagatcgagctggccgtgctgctgtccaacgagggcatcatcaacagcgaggacgagcatctgctggccctggaacggaagctgaagaagatgctgggccctagcgccgtggacatcggcaacggctgcttcgagacaaagcacaagtgcaaccagacctgcctggaccggatcgctgccggcaccttcaacgccggcgagttcagcctgcccaccttcgacagcctgaacatcaccgccgccagcctgaacgacgacggcctggacaaccacaccatcctgctgtactacagcaccgcagcctccagcctggccgtgaccctgatgatcgccatcttcatcgtgtacatggtgtctcgggacaacgtgtcctgcagcatctgcctg

SEQ ID NO: 10 - protein, 583 aa, Artificial Sequence
  protein type B hemagluttinin
KAIIVLLMVVTSNADRICTGITSSNSPHVVKTATQGEVNVTGVIPLTTTPTKSHFANLKGTKTRGKLCPKCLNCTDLDVALGRPMCVGTTPSAKASILHEVRPVTSGCFPIMHDRTKIRQLPNLLRGYENIRLSTQNVINAEKAPGGPYRLGTSGSCPNATSKSGFFATMAWAVPKDNNKTATNPLTVEVPYICTEGEDQITVWGFHSDNKTQMKNLYGDSNPQKFTSSANGVTTHYVSQIGGFPDQTEDGGLPQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 11 - DNA, 2142 bp, Artificial Sequence
  Subtype A consensus Envelope DNA sequence construct
ggatccatggactggacctggattctgttcctggtggccgccgccaccagagtgcacagcagagtgatgggcatccagcggaattgccagcacctgtggagatggggcaccatgatcctgggcatgatcatcatctgctctgccgccgagaacctgtgggtgaccgtgtactacggcgtgcctgtgtggaaggacgccgagaccaccctgttctgcgccagcgacgccaaggcctacgataccgaagtgcacaatgtgtgggccacccacgcctgcgtgcctaccgatcccaacccccaggagatcaacctggagaacgtgaccgaggagttcaacatgtggaagaacaacatggtggagcagatgcacaccgacatcatcagcctgtgggaccagagcctgaagccttgcgtgaagctgacccctctgtgcgtgaccctgaactgcagcaacgtgaacgtgaccaccaacatcatgaagggcgagatcaagaactgcagcttcaacatgaccaccgagctgcgggacaagaagcagaaagtgtacagcctgttctacaagctggacgtggtgcagatcaacaagagcaacagcagcagccagtaccggctgatcaactgcaacaccagcgccatcacccaggcctgccccaaagtgagcttcgagcccatccccatccactactgcgcccctgccggcttcgccatcctgaagtgcaaggacaaggagtttaacggcaccggcccctgcaagaatgtgagcaccgtgcagtgcacccacggcatcaagcccgtggtgtccacccagctgctgctgaacggcagcctggccgaggaggaagtgatgatccggagcgagaacatcaccaacaacgccaagaacatcatcgtgcagctgaccaagcccgtgaagatcaattgcacccggcccaacaacaacacccggaagagcatcagaatcggccctggccaggccttctacgccaccggcgacatcatcggcgatatcaggcaggcccactgcaatgtgagccggaccgagtggaacgagaccctgcagaaagtggccaagcagctgcggaagtacttcaacaacaagaccatcatcttcaccaacagcagcggcggcagactgagaatcaccacccacagcttcaattgtggcggcgagttcttctactgcaatacctccggcctgttcaacagcacctggaacggcaacggcaccaagaagaagaacagcaccgagagcaacgacaccatcaccctgccctgccggatcaagcagatcatcaatatgtggcagagggtgggccaggccatgtacgcccctcccatccagggcgtgatcagatgcgagagcaacatcaccggcctgctgctgaccagagatggcggcgacaacaacagcaagaacgagaccttcagacctggcggcggagacatgagggacaactggcggagcgagctgtacaagtacaaagtggtgaagatcgagcccctgggcgtggcccccaccaaggccaagagaagagtggtggagcgggagaagagagctgtgggcatcggcgccgtgttcctgggcttcctgggagccgccggaagcaccatgggagccgccagcatcaccctgaccgtgcaggccagacagctgctgagcggcattgtgcagcagcagagcaacctgctgagagccatcgaggcccagcagcacctgctgaagctgacagtgtggggcatcaaacagctgcaggcccgcgtgctggccgtggagagatacctgaaggaccagcagctgctgggcatctggggctgcagcggcaagctgatctgcaccaccaacgtgccctggaatagcagctggagcaacaagagccagagcgagatctgggacaacatgacctggctgcagtgggacaaggagatcagcaactacaccgatatcatctacaacctgatcgaggagagccagaaccagcaggagaagaacgagcaggatctgctggccctggacaagtgggccaacctgtggaactggttcgacatcagcaactggctgtggtacatcaagatcttcatcatgattgtgggcggcctgatcggcctgagaatcgtgttcgccgtgctgtctgtgtgactcgag

SEQ ID NO: 12 - protein, 709 aa, Artificial Sequence
  Subtype A consensus Envelope protein sequence construct
MDWTWILFLVAAATRVHSRVMGIQRNCQHLWRWGTMILGMIIICSAAENLWVTVYYGVPVWKDAETTLFCASDAKAYDTEVHNVWATHACVPTDPNPQEINLENVTEEFNMWKNNMVEQMHTDIISLWDQSLKPCVKLTPLCVTLNCSNVNVTTNIMKGEIKNCSFNMTTELRDKKQKVYSLFYKLDVVQINKSNSSSQYRLINCNTSAITQACPKVSFEPIPIHYCAPAGFAILKCKDKEFNGTGPCKNVSTVQCTHGIKPVVSTQLLLNGSLAEEEVMIRSENITNNAKNIIVQLTKPVKINCTRPNNNTRKSIRIGPGQAFYATGDIIGDIRQAHCNVSRTEWNETLQKVAKQLRKYFNNKTIIFTNSSGGRLRITTHSFNCGGEFFYCNTSGLFNSTWNGNGTKKKNSTESNDTITLPCRIKQIINMWQRVGQAMYAPPIQGVIRCESNITGLLLTRDGGDNNSKNETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQSEIWDNMTWLQWDKEISNYTDIIYNLIEESQNQQEKNEQDLLALDKWANLWNWFDISNWLWYIKIFIMIVGGLIGLRIVFAVLSV

SEQ ID NO: 13 - DNA, 2734 bp, Artificial Sequence
  Subtype B consensus Envelope DNA sequence construct
ggatccgccaccatggactggacctggattctgttcctggtggccgccgccaccagagtgcacagcagagtgaagggcatccggaagaactaccagcacctgtggagatggggcaccatgctgctgggcatgctgatgatctgttctgccgccgagaagctgtgggtgaccgtgtactacggcgtgcctgtgtggaaggaggccaccaccaccctgttctgcgccagcgacgccaaggcctacgataccgaagtgcacaatgtgtgggccacccacgcctgcgtgcctaccgatcccaaccctcaggaagtggtgctggagaacgtgaccgagaacttcaacatgtggaagaacaacatggtggagcagatgcacgaggacatcatcagcctgtgggaccagagcctgaagccttgcgtgaagctgacccctctgtgcgtgaccctgaactgcaccgacctgagcggcgagaagatggagaagggcgagatcaagaactgcagcttcaacatcaccacctccatccgggacaaagtgcagaaggagtacgccctgttctacaagctggacgtggtgcccatcgacaacgacaacaccagctaccggctgatcagctgcaacaccagcgtgatcacccaggcctgccccaaagtgagcttcgagcccatccccatccactactgcgcccctgccggcttcgccatcctgaagtgcaacgacaagaagttcaacggcaccggcccttgcaccaatgtgagcaccgtgcagtgcacccacggcatcagacccgtggtgtccacccagctgctgctgaacggcagcctggccgaggaagaagtggtgatccggagcgagaatttcaccaacaacgccaagaccatcatcgtgcagctgaacgagagcgtggagatcaactgcacccggcccaacaacaatacccggaagagcatccacatcggccctggccaggccttctacaccaccggcgagatcatcggcgatatcaggcaggcccactgcaatatcagccgggccaagtggaacaacaccctgaagcagatcgtgaagaagctgcgggagcagttcggcaacaagaccatcgtgttcaaccagagcagcggcggcagacctagaatcgtgatgcacagcttcaactgtggcggcgagttcttctactgcaacacaacccagctgttcaacagcacctggaacgtgaacgggacctggaacaacaacaccgagggcaacgacaccatcaccctgccctgccggatcaagcagatcatcaatatgtggcaggaggtgggcaaggccatgtacgcccctcccatcagaggccagatccggtgcagcagcaatatcaccggcctgctgctgaccagagatggcggcaacaataacaccaacgagaccgagatctttagacctggcggcggagacatgagggacaactggcggagcgagctgtacaagtacaaagtggtgaagatcgagcccctgggcgtggcccccaccaaggccaagagaagagtggtgcagcgggagaagagagctgtgggcatcggcgccatgtttctgggctttctgggagccgccggaagcaccatgggagccgccagcatgaccctgaccgtgcaggccagacagctgctgagcggcatcgtgcagcagcagaacaacctgctgagagccatcgaggcccagcagcacctgctgcagctgacagtgtggggcatcaagcagctgcaggcccgcgtgctggccgtggagagatacctgaaggaccagcagctgctgggaatctggggctgcagcggcaagctgatctgcaccaccaccgtgccctggaacgccagctggagcaacaagagcctggacgagatctgggacaacatgacctggatggagtgggagcgggagatcgacaactacaccagcctgatctacaccctgatcgaggagagccagaaccagcaggagaagaacgagcaggagctgctggagctggacaagtgggccagcctgtggaactggttcgacatcaccaactggctgtggtacatcaagatcttcatcatgattgtgggcggcctgatcggcctgagaatcgtgttcgccgtgctgagcatctacccctacgacgtgcccgattacgcctgagaattcgtaagtaagtgtcatatgggagagctcgactagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctaccggtatcgatgttaacgtcgaccccgggctcgaggtaagtaagtgtcatatgggagagctcgactagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctgcggccgc

SEQ ID NO: 14 - protein, 715 aa, Artificial Sequence
  Subtype B consensus Envelope protein sequence construct
MDWTWILFLVAAATRVHSRVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLSGEKMEKGEIKNCSFNITTSIRDKVQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTNNAKTIIVQLNESVEINCTRPNNNTRKSIHIGPGQAFYTTGEIIGDIRQAHCNISRAKWNNTLKQIVKKLREQFGNKTIVFNQSSGGRPRIVMHSFNCGGEFFYCNTTQLFNSTWNVNGTWNNNTEGNDTITLPCRIKQIINMWQEVGKAMYAPPIRGQIRCSSNITGLLLTRDGGNNNTNETEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTTVPWNASWSNKSLDEIWDNMTWMEWEREIDNYTSLIYTLIEESQNQQEKNEQELLELDKWASLWNWFDITNWLWYIKIFIMIVGGLIGLRIVFAVLSIYPYDVPDYA

SEQ ID NO: 15 - DNA, 2140 bp, Artificial Sequence
  Subtype C consensus Envelope DNA sequence construct
ggatccgccaccatggattggacctggattctgttcctggtggccgccgccacaagagtgcacagcagagtgcggggcatcctgagaaattgccagcagtggtggatctggggcattctggggttctggatgctgatgatctgcaacgtgatgggcaacctgtgggtgaccgtgtactacggcgtgcctgtgtggaaggaggccaagaccaccctgttctgtgccagcgatgccaaggcctacgagaccgaggtgcacaatgtgtgggccacccacgcctgtgtgcccaccgatcccaaccctcaggagatggtgctggagaacgtgaccgagaacttcaacatgtggaagaacgacatggtggaccagatgcacgaggacatcatcagcctgtgggaccagagcctgaagccttgcgtgaagctgacccctctgtgcgtgaccctgaactgccggaacaacgtgaacaacaacaacaccatgaaggaggagatcaagaactgcagcttcaacatcaccaccgagctgcgggacaagaagcagaaggtgtacgccctgttctaccggctggacatcgtgcccctgaacgagaagaacaacagcaacgactaccggctgatcaactgcaacaccagcgccatcacccaggcctgtcccaaggtgtccttcgaccccatccccatccactattgtgcccctgccggctacgccatcctgaagtgcaacaacaagaccttcaacggcaccggcccctgcaataatgtgagcaccgtgcagtgtacccacggcatcaagcctgtggtgtccacccagctgctgctgaatggcagcctggccgaggaggagattatcatccggagcgagaacctgaccaacaacgccaagaccatcattgtgcacctgaatgagagcgtggagatcgtgtgtacccggcccaacaacaatacccggaagagcatcagaatcggccctggccagaccttttacgccaccggcgacatcatcggcgatatcaggcaggcccactgcaatatcagcgaggagaagtggaacaagaccctgcagcgggtgtccgagaagctgaaggagcacttccccaataagaccatcaagttcgcccctagcagcggcggcagactggagatcaccacccacagcttcaactgcaggggcgagttcttctactgcaataccagcaagctgttcaacagcacctacatgcccaacagcaccaacaataccaacaccaccatcaccctgccctgccggatcaagcagatcatcaatatgtggcaggaagtgggcagagccatgtacgcccctcccatcgagggcaacatcacctgcaagtccaacatcaccggcctgctgctgacaagagatggcggcaagaacgacaccaatgacaccgagaccttcagacctggcggcggagacatgagggacaactggcggagcgagctgtacaagtacaaggtggtggagatcaagcctctgggcgtggcccctaccaaggccaagaggagagtggtggagagggagaagagagccgtgggcatcggcgccgtgtttctgggctttctgggagccgccggatctacaatgggagccgccagcatcacactgaccgtgcaggccagacagctgctgagcggcatcgtgcagcagcagagcaatctgctgagagccatcgaggcccagcagcacatgctgcagctgacagtgtggggcatcaagcagctgcagaccagagtgctggccatcgagcgctacctgaaggatcagcagctgctgggcatctggggctgtagcggcaagctgatctgtaccaccgccgtgccttggaatagcagctggagcaacaagagccaggaggacatctgggacaacatgacctggatgcagtgggaccgggagatcagcaactacaccgacaccatctacaggctgctggaggacagccagaaccagcaggagaagaacgagaaggacctgctggccctggacagctggaagaacctgtggaactggttcgacatcaccaactggctgtggtacatcaagatcttcatcatgattgtgggcggcctgatcggcctgagaatcatcttcgccgtgctgagcatctgatagcggccgc

SEQ ID NO: 16 - protein, 705 aa, Artificial Sequence
  Subtype C consensus Envelope protein sequence construct
MDWTWILFLVAAATRVHSRVRGILRNCQQWWIWGILGFWMLMICNVMGNLWVTVYYGVPVWKEAKTTLFCASDAKAYETEVHNVWATHACVPTDPNPQEMVLENVTENFNMWKNDMVDQMHEDIISLWDQSLKPCVKLTPLCVTLNCRNNVNNNNTMKEEIKNCSFNITTELRDKKQKVYALFYRLDIVPLNEKNNSNDYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEEKWNKTLQRVSEKLKEHFPNKTIKFAPSSGGRLEITTHSFNCRGEFFYCNTSKLFNSTYMPNSTNNTNTTITLPCRIKQIINMWQEVGRAMYAPPIEGNITCKSNITGLLLTRDGGKNDTNDTETFRPGGGDMRDNWRSELYKYKVVEIKPLGVAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSI

SEQ ID NO: 17 - DNA, 2089 bp, Artificial Sequence
  Subtype D consensus Envelope DNA sequence construct
gggcatcaagcggaattaccagcacctgtggaagtggggcaccatgctgctgggcatgctgatgacctgcagcgtggccgagaacctgtgggtgaccgtgtactacggcgtgcctgtgtggaaggaagccaccaccaccctgttctgcgccagcgatgccaagagctacaagaccgaggcccacaatatctgggccacccacgcctgcgtgcctaccgatcccaaccctcaggagatcgagctggagaacgtgaccgagaacttcaacatgtggaagaacaacatggtggagcagatgcacgaggacatcatcagcctgtgggaccagagcctgaagccttgcgtgaagctgacccctctgtgcgtgaccctgaactgcaccgacggcatgaggaacgacaccaacgataccaacgtgaccatggaggagggcgagatgaagaactgcagcttcaacatcaccaccgaagtgcgggacaagaagaagcaggtgcacgccctgttctacaagctggacgtggtgcccatcgacgacaacaacaccaacaacagcaactaccggctgatcaactgcaacaccagcgccatcacccaggcctgccccaaagtgaccttcgagcccatccccatccactactgcgcccctgccggcttcgccatcctgaagtgcaaggataagaagttcaacggcaccggcccctgcaagaatgtgagcaccgtgcagtgcacccacggcatcagacccgtggtgtccacccagctgctgctgaacggcagcctggccgaggaggagatcatcatccggagcgagaacctgaccaacaacgccaagatcatcattgtgcagctgaacgagagcgtgaccatcaattgcacccggccctacaacaatacccggaagcgcatccccatcggcctgggccaggccttctacaccaccagaggcatcatcggcgacatcagacaggcccactgcaatatcagcggagccgagtggaataagaccctgcagcaggtggccaagaagctgggcgacctgctgaacaagaccaccatcatcttcaagcctagcagcggcggcagacctagaatcaccacccacagcttcaattgtggcggcgagttcttctactgcaataccagccggctgttcaacagcacctggagcaagaacagcaccagcaactccaccaaggagaacaacaccatcaccctgccctgccggatcaagcagatcatcaatatgtggcagggagtgggcaaggccatgtacgcccctcccatcgagggcctgatcaagtgcagcagcaacatcaccggcctgctgctgaccagagatggcggagccaacaactcccacaacgagaccttcagacctggcggcggagacatgagggacaactggcggagcgagctgtacaagtacaaagtggtgaagatcgagcccctgggcgtggcccccaccagagccaagagaagagtggtggagcgggagaagagagccatcggactgggcgccatgttcctgggcttcctgggagccgccggaagcaccatgggagccgccagcctgaccctgaccgtgcaggccagacagctgctgagcggcatcgtgcagcagcagaacaacctgctgagagccattgaggcccagcagcacctgctgcagctgacagtgtggggcattaagcagctgcaggccaggattctggccgtggagcgctacctgaaggatcagcagctgctgggaatctggggctgcagcggcaagcacatctgcaccaccaccgtgccttggaatagcagctggagcaacaagagcctggacgagatctggaacaacatgacctggatggagtgggagagggagatcgacaactacaccggcctgatctacagcctgatcgaggagagccagacccagcaggagaagaacgagcaggagctgctggagctggacaagtgggccagcctgtggaactggttcagcatcacccagtggctgtggtacatcaagatcttcatcatgattgtgggcggcctgatcggcctgagaatcgtgttcgccgtgctgagcctgtgactcgag

SEQ ID NO: 18 - protein, 714 aa, Artificial Sequence
  Subtype D consensus Envelope protein sequence construct
MDWTWILFLVAAATRVHSRVRGIKRNYQHLWKWGTMLLGMLMTCSVAENLWVTVYYGVPVWKEATTTLFCASDAKSYKTEAHNIWATHACVPTDPNPQEIELENVTENFNMWKNNMVEQMHEDIISLWDQSLKPCVKLTPLCVTLNCTDGMRNDTNDTNVTMEEGEMKNCSFNITTEVRDKKKQVHALFYKLDVVPIDDNNTNNSNYRLINCNTSAITQACPKVTFEPIPIHYCAPAGFAILKCKDKKFNGTGPCKNVSTVQCTHGIRPVVSTQLLLNGSLAEEEIIIRSENLTNNAKIIIVQLNESVTINCTRPYNNTRKRIPIGLGQAFYTTRGIIGDIRQAHCNISGAEWNKTLQQVAKKLGDLLNKTTIIFKPSSGGRPRITTHSFNCGGEFFYCNTSRLFNSTWSKNSTSNSTKENNTITLPCRIKQIINMWQGVGKAMYAPPIEGLIKCSSNITGLLLTRDGGANNSHNETFRPGGGDMRDNWRSELYKYKVVKIEPLGVAPTRAKRRVVEREKRAIGLGAMFLGFLGAAGSTMGAASLTLTVQARQLLSGIVQQQNNLLRAIEAQQHLLQLTVWGIKQLQARILAVERYLKDQQLLGIWGCSGKHICTTTVPWNSSWSNKSLDEIWNNMTWMEWEREIDNYTGLIYSLIEESQTQQEKNEQELLELDKWASLWNWFSITQWLWYIKIFIMIVGGLIGLRIVFAVLSL

SEQ ID NO: 19 - DNA, 1049 bp, Artificial Sequence
  Subtype B consensus Nef-Rev DNA sequence construct
ggatccgccaccatggactggacctggattctgttcctggtggccgctgccaccagagtgcacagcagcaagagaagcgtggtgggttggcctacagtgcgggagaggatgagaagagccgagcctgccgccgatggagtgggcgccgtgtctagagatctggagaagcacggcgccatcaccagcagcaataccgccgccaacaatgccgactgcgcctggctggaggcccaggaggaggaggaagtgggcttccctgtgagagcccaggtggccctgagagccatgacctacaaggccgccgtggatctgagccacttcctgaaggagaagggcggcctggagggcctgatctacagccagaagcggcaggacatcctggatctgtgggtgtaccacacccagggctacttccccgactggcagaattacacccctggccctggcatcagataccctctgaccttcggctggtgcttcaagctggtgcctgtggagcctgagaaagtggaggaggccaacgagggcgagaacaattctgccgcccaccctatgagcctgcacggcatggacgatcccgagagggaagtgctggtgtggaagttcgacagcaggctggccttccaccacatggccagagagctgcaccccgagtactacaaggactgccggggcaggaagagaagaagcgccggcagaagcggcgacagcgacgaggagctgctgaaaacagtgcggctgatcaagttcctgtaccagagcaaccctcctcccagccccgagggcaccagacaggcccggagaaaccggaggaggcggtggagagagaggcagcggcagatcagaagcatcagcgagtggattctgagcacctacctgggcagacccgccgagcccgtgcccctgcagctgccccccctggagagactgaccctggactgcaacgaggactgcggcaccagcggcacccagggagtgggcagcccccagatcctggtggagagccctgccgtgctggagagcggcaccaaggagtgatgagcggccgc

SEQ ID NO: 20 - protein, 341 aa, Artificial Sequence
  Subtype B consensus Nef-Rev protein sequence construct
MDWTWILFLVAAATRVHSSKRSVVGWPTVRERMRRAEPAADGVGAVSRDLEKHGAITSSNTAANNADCAWLEAQEEEEVGFPVRAQVALRAMTYKAAVDLSHFLKEKGGLEGLIYSQKRQDILDLWVYHTQGYFPDWQNYTPGPGIRYPLTFGWCFKLVPVEPEKVEEANEGENNSAAHPMSLHGMDDPEREVLVWKFDSRLAFHHMARELHPEYYKDCRGRKRRSAGRSGDSDEELLKTVRLIKFLYQSNPPPSPEGTRQARRNRRRRWRERQRQIRSISEWILSTYLGRPAEPVPLQLPPLERLTLDCNEDCGTSGTQGVGSPQILVESPAVLESGTKE

SEQ ID NO: 21 - DNA, 1863 bp, Artificial Sequence
  Gag consensus DNA sequence of subtype A, B, C and D construct
ggatccgccaccatggactggacctggattctgtttctggtcgccgccgccacaagagtgcacagcggcgccagagccagcgtgctgtccggcggcaagctggacgcctgggagaagatcagactgaggcctggcggcaagaagaagtaccggctgaagcaccttgtgtgggccagcagagagctggagagattcgccctgaatcctggcctgctggagaccagcgagggctgtaagcagatcatcggccagctgcagcccgccctgcagaccggcagcgaggagctgagaagcctgtacaacaccgtggccaccctgtactgcgtgcacgagaagatcgaggtgaaggacaccaaggaggccctggacaagatcgaggaggagcagaacaagagcaagcagaaggcccagcaggccgccgccgacaccggcaacagcagccaggtgtcccagaactaccccatcgtgcagaatctgcagggccagatggtgcaccaggccatcagccccagaaccctgaatgcctgggtgaaggtgatcgaggagaaggccttcagccctgaggtgatccctatgttcagcgccctgagcgagggcgccacacctcaggacctgaacaccatgctgaacacagtggggggccaccaggccgccatgcagatgctgaaggataccatcaacgaggaggccgccgagtgggacagactgcaccccgtgcacgccggacctatcgcccctggccagatgagagagcccagaggcagcgacatcgccggcaccacctccaccctgcaagagcagatcggctggatgaccagcaacccccccatccctgtgggcgacatctacaagcggtggatcatcctgggcctgaacaagattgtgaggatgtacagccccgtgtccatcctggatatcaggcagggccccaaggagcccttcagagactacgtggaccggttcttcaagaccctgagagccgagcaggccagccaggacgtgaagaactggatgaccgagaccctgctggtgcagaacgccaaccccgactgtaagaccatcctgagagccctgggccctggcgccaccctggaggagatgatgaccgcctgccagggagtgggcggacccggccacaaggccagagtgctggccgaggccatgagccaggccaccaacagcaacatcatgatgcagcggggcaacttcagaggccccaggaggatcgtgaagtgcttcaactgtggcaaggagggccacatcgccagaaactgtagggcccccaggaagaagggctgctggaagtgtggcaaagaggggcaccagatgaaggactgtaccgagcggcaggccaatttcctggggaagatctggcccagccacaagggcagacccggcaatttcctgcagagcagacctgagcccaccgcccctcccgccgagagcttcggcttcggcgaggagatcacccccagccccaagcaggagcccaaggacagagagctgtaccctctggccagcctgaagagcctgttcggcaacgatcccctgagccagtacccctacgacgtgcccgattacgcctgagaattcgtaagtaagtgtcatatgggagagctcgactagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctgcggccgc

SEQ ID NO: 22 - protein, 524 aa, Artificial Sequence
  Gag consensus protein sequence of subtype A, B, C and D construct
MDWTWILFLVAAATRVHSGARASVLSGGKLDAWEKIRLRPGGKKKYRLKHLVWASRELERFALNPGLLETSEGCKQIIGQLQPALQTGSEELRSLYNTVATLYCVHEKIEVKDTKEALDKIEEEQNKSKQKAQQAAADTGNSSQVSQNYPIVQNLQGQMVHQAISPRTLNAWVKVIEEKAFSPEVIPMFSALSEGATPQDLNTMLNTVGGHQAAMQMLKDTINEEAAEWDRLHPVHAGPIAPGQMREPRGSDIAGTTSTLQEQIGWMTSNPPIPVGDIYKRWIILGLNKIVRMYSPVSILDIRQGPKEPFRDYVDRFFKTLRAEQASQDVKNWMTETLLVQNANPDCKTILRALGPGATLEEMMTACQGVGGPGHKARVLAEAMSQATNSNIMMQRGNFRGPRRIVKCFNCGKEGHIARNCRAPRKKGCWKCGKEGHQMKDCTERQANFLGKIWPSHKGRPGNFLQSRPEPTAPPAESFGFGEEITPSPKQEPKDRELYPLASLKSLFGNDPLSQYPYDVPDYA

SEQ ID NO: 23 - protein, 692 aa, Artificial Sequence
  Subtype A consensus Envelope protein sequence
SRVMGIQRNCQHLWRWGTMILGMIIICSAAENLWVTVYYGVPVWKDAETTLFCASDAKAYDTEVHNVWATHACVPTDPNPQEINLENVTEEFNMWKNNMVEQMHTDIISLWDQSLKPCVKLTPLCVTLNCSNVNVTTNIMKGEIKNCSFNMTTELRDKKQKVYSLFYKLDVVQINKSNSSSQYRLINCNTSAITQACPKVSFEPIPIHYCAPAGFAILKCKDKEFNGTGPCKNVSTVQCTHGIKPVVSTQLLLNGSLAEEEVMIRSENITNNAKNIIVQLTKPVKINCTRPNNNTRKSIRIGPGQAFYATGDIIGDIRQAHCNVSRTEWNETLQKVAKQLRKYFNNKTIIFTNSSGGRLRITTHSFNCGGEFFYCNTSGLFNSTWNGNGTKKKNSTESNDTITLPCRIKQIINMWQRVGQAMYAPPIQGVIRCESNITGLLLTRDGGDNNSKNETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQSEIWDNMTWLQWDKEISNYTDIIYNLIEESQNQQEKNEQDLLALDKWANLWNWFDISNWLWYIKIFIMIVGGLIGLRIVFAVLSV

SEQ ID NO: 24 - protein, 697 aa, Artificial Sequence
  Subtype B consensus Envelope protein sequence
RVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLSGEKMEKGEIKNCSFNITTSIRDKVQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTNNAKTIIVQLNESVEINCTRPNNNTRKSIHIGPGQAFYTTGEIIGDIRQAHCNISRAKWNNTLKQIVKKLREQFGNKTIVFNQSSGGRPRIVMHSFNCGGEFFYCNTTQLFNSTWNVNGTWNNNTEGNDTITLPCRIKQIINMWQEVGKAMYAPPIRGQIRCSSNITGLLLTRDGGNNNTNETEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTTVPWNASWSNKSLDEIWDNMTWMEWEREIDNYTSLIYTLIEESQNQQEKNEQELLELDKWASLWNWFDITNWLWYIKIFIMIVGGLIGLRIVFAVLSIYPYDVPDYA

SEQ ID NO: 25 - protein, 687 aa, Artificial Sequence
  Subtype C consensus Envelope protein sequence
RVRGILRNCQQWWIWGILGFWMLMICNVMGNLWVTVYYGVPVWKEAKTTLFCASDAKAYETEVHNVWATHACVPTDPNPQEMVLENVTENFNMWKNDMVDQMHEDIISLWDQSLKPCVKLTPLCVTLNCRNNVNNNNTMKEEIKNCSFNITTELRDKKQKVYALFYRLDIVPLNEKNNSNDYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEEKWNKTLQRVSEKLKEHFPNKTIKFAPSSGGRLEITTHSFNCRGEFFYCNTSKLFNSTYMPNSTNNTNTTITLPCRIKQIINMWQEVGRAMYAPPIEGNITCKSNITGLLLTRDGGKNDTNDTETFRPGGGDMRDNWRSELYKYKVVEIKPLGVAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSI

SEQ ID NO: 26 - protein, 696 aa, Artificial Sequence
  Subtype D consensus Envelope protein sequence
RVRGIKRNYQHLWKWGTMLLGMLMTCSVAENLWVTVYYGVPVWKEATTTLFCASDAKSYKTEAHNIWATHACVPTDPNPQEIELENVTENFNMWKNNMVEQMHEDIISLWDQSLKPCVKLTPLCVTLNCTDGMRNDTNDTNVTMEEGEMKNCSFNITTEVRDKKKQVHALFYKLDVVPIDDNNTNNSNYRLINCNTSAITQACPKVTFEPIPIHYCAPAGFAILKCKDKKFNGTGPCKNVSTVQCTHGIRPVVSTQLLLNGSLAEEEIIIRSENLTNNAKIIIVQLNESVTINCTRPYNNTRKRIPIGLGQAFYTTRGIIGDIRQAHCNISGAEWNKTLQQVAKKLGDLLNKTTIIFKPSSGGRPRITTHSFNCGGEFFYCNTSRLFNSTWSKNSTSNSTKENNTITLPCRIKQIINMWQGVGKAMYAPPIEGLIKCSSNITGLLLTRDGGANNSHNETFRPGGGDMRDNWRSELYKYKVVKIEPLGVAPTRAKRRVVEREKRAIGLGAMFLGFLGAAGSTMGAASLTLTVQARQLLSGIVQQQNNLLRAIEAQQHLLQLTVWGIKQLQARILAVERYLKDQQLLGIWGCSGKHICTTTVPWNSSWSNKSLDEIWNNMTWMEWEREIDNYTGLIYSLIEESQTQQEKNEQELLELDKWASLWNWFSITQWLWYIKIFIMIVGGLIGLRIVFAVLSL

SEQ ID NO: 27 - protein, 323 aa, Artificial Sequence
  Subtype B consensus Nef-Rev protein sequence
SKRSVVGWPTVRERMRRAEPAADGVGAVSRDLEKHGAITSSNTAANNADCAWLEAQEEEEVGFPVRAQVALRAMTYKAAVDLSHFLKEKGGLEGLIYSQKRQDILDLWVYHTQGYFPDWQNYTPGPGIRYPLTFGWCFKLVPVEPEKVEEANEGENNSAAHPMSLHGMDDPEREVLVWKFDSRLAFHHMARELHPEYYKDCRGRKRRSAGRSGDSDEELLKTVRLIKFLYQSNPPPSPEGTRQARRNRRRRWRERQRQIRSISEWILSTYLGRPAEPVPLQLPPLERLTLDCNEDCGTSGTQGVGSPQILVESPAVLESGTKE

SEQ ID NO: 28 - protein, 506 aa, Artificial Sequence
  Gag consensus protein sequence of subtype A, B, C and D
GARASVLSGGKLDAWEKIRLRPGGKKKYRLKHLVWASRELERFALNPGLLETSEGCKQIIGQLQPALQTGSEELRSLYNTVATLYCVHEKIEVKDTKEALDKIEEEQNKSKQKAQQAAADTGNSSQVSQNYPIVQNLQGQMVHQAISPRTLNAWVKVIEEKAFSPEVIPMFSALSEGATPQDLNTMLNTVGGHQAAMQMLKDTINEEAAEWDRLHPVHAGPIAPGQMREPRGSDIAGTTSTLQEQIGWMTSNPPIPVGDIYKRWIILGLNKIVRMYSPVSILDIRQGPKEPFRDYVDRFFKTLRAEQASQDVKNWMTETLLVQNANPDCKTILRALGPGATLEEMMTACQGVGGPGHKARVLAEAMSQATNSNIMMQRGNFRGPRRIVKCFNCGKEGHIARNCRAPRKKGCWKCGKEGHQMKDCTERQANFLGKIWPSHKGRPGNFLQSRPEPTAPPAESFGFGEEITPSPKQEPKDRELYPLASLKSLFGNDPLSQYPYDVPDYA

SEQ ID NO: 29 - DNA, 1707 bp, Artificial Sequence
  Influenza H5N1 HA consensus sequence
atggaaaagatcgtgctgctgttcgccatcgtgagcctggtgaagagcgaccagatctgcatcggctaccacgccaacaacagcaccgagcaggtggacaccatcatggaaaaaaacgtgaccgtgacccacgcccaggacatcctggaaaagacccacaacggcaagctgtgcgacctggacggcgtgaagcccctgatcctgcgggactgcagcgtggccggctggctgctgggcaaccccatgtgcgacgagttcatcaacgtgcccgagtggagctacatcgtggagaaggccaaccccgtgaacgacctgtgctaccccggcgacttcaacgactacgaggaactgaagcacctgctgtcccggatcaaccacttcgagaagatccagatcatccccaagagcagctggtccagccacgaggccagcctgggcgtgagcagcgcctgcccataccagggcaagtccagcttcttccggaacgtggtgtggctgatcaagaagaacagcacctaccccaccatcaagcggagctacaacaacaccaaccaggaagatctgctggtcctgtggggcatccaccaccccaacgacgccgccgagcagaccaagctgtaccagaaccccaccacctacatcagcgtgggcaccagcaccctgaaccagcggctggtgccccggatcgccacccggtccaaggtgaacggccagagcggccggatggaattcttctggaccatcctgaagcccaacgatgccatcaacttcgagagcaacggcaacttcatcgcccccgagtacgcctacaagatcgtgaagaagggcgacagcaccatcatgaagagcgagctggaatacggcaactgcaacaccaagtgccagacccccatgggcgccatcaacagcagcatgcccttccacaacatccaccccctgaccatcggcgagtgccccaagtacgtgaagagcaacaggctggtgctggccaccggcctgcggaacagcccccagcgggagcggcgggccgccgcccggggcctgttcggcgccatcgccggcttcatcgagggcggctggcagggcatggtggacgggtggtacggctaccaccacagcaatgagcagggcagcggctacgccgccgacaaagagagcacccagaaggccatcgacggcgtcaccaacaaggtgaacagcatcatcgacaagatgaacacccagttcgaggccgtgggccgggagttcaacaacctggaacggcggatcgagaacctgaacaagaaaatggaagatggcttcctggacgtgtggacctacaacgccgagctgctggtgctgatggaaaacgagcggaccctggacttccacgacagcaacgtgaagaacctgtacgacaaagtgcggctgcagctgcgggacaacgccaaagagctgggcaacggctgcttcgagttctaccacaagtgcgacaacgagtgcatggaaagcgtgcggaacggcacctacgactacccccagtacagcgaggaagcccggctgaagcgggaggaaatcagcggcgtgaaactggaaagcatcggcatctaccagatcctgagcatctacagcaccgtggccagcagcctggccctggccatcatggtggccggcctgagcctgtggatgtgcagcaacggcagcctgcagtgccggatctgcatctag

SEQ ID NO: 30 - protein, 567 aa, Artificial Sequence
  Influenza H5N1 HA consensus sequence
EKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRAAARGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 31 - DNA, 1466 bp, Artificial Sequence
  Influenza H1N1&H5N1 NA consensus Sequence
ggtaccgaattcgccaccatggactggacctggatcctgttcctggtggccgctgccacccgggtgcacagcatgaaccccaaccagaagatcatcaccatcggcagcatctgcatggtgatcggcatcgtgagcctgatgctgcagatcggcaacatgatcagcatctgggtgtcccacagcatccagaccggcaaccagcaccaggccgagcccatcagcaacaccaactttctgaccgagaaggccgtggccagcgtgaccctggccggcaacagcagcctgtgccccatcagcggctgggccgtgtacagcaaggacaacagcatccggatcggcagcaagggcgacgtgttcgtgatccgggagcccttcatcagctgcagccacctggaatgccggaccttcttcctgacccagggggccctgctgaacgacaagcacagcaacggcaccgtgaaggacagaagcccctaccggaccctgatgagctgccccgtgggcgaggcccccagcccctacaacagccggttcgagagcgtggcctggtccgccagcgcctgccacgacggcaccagctggctgaccatcggcatcagcggccctgacaacggcgccgtggccgtgctgaagtacaacggcatcatcaccgacaccatcaagagctggcggaacaacatcctgcggacccaggaaagcgagtgcgcctgcgtgaacggcagctgcttcaccgtgatgaccgacggccccagcaacggccaggccagctacaagatcttcaagatggaaaagggcaaggtggtgaagagcgtggagctggacgcccccaactaccactacgaggaatgcagctgctaccccgacgccggcgagatcacctgcgtgtgccgggacaactggcacggcagcaaccggccctgggtgtccttcaaccagaacctggaataccagatcggctacatctgcagcggcgtgttcggcgacaaccccaggcccaacgatggcaccggcagctgcggccctgtgagcgccaacggcgcctacggcgtgaagggcttcagcttcaagtacggcaacggcgtgtggatcggccggaccaagagcaccaacagcagatccggcttcgagatgatctgggaccccaacggctggaccgagaccgacagcagcttcagcgtgaagcaggacatcgtggccatcaccgactggtccggctacagcggcagcttcgtgcagcaccccgagctgaccggcctggactgcatccggccctgcttttgggtggagctgatcagaggcaggcccaaagagagcaccatctggaccagcggcagcagcatcagcttttgcggcgtgaacagcgacaccgtgagctggtcctggcccgacggcgccgagctgcccttcaccatcgacaagtacccctacgacgtgcccgactacgcctgatgagcggccgcgagctc

SEQ ID NO: 32 - protein, 476 aa, Artificial Sequence
  Influenza H1N1&H5N1 NA consensus protein Sequence
MDWTWILFLVAAATRVHSMNPNQKIITIGSICMVIGIVSLMLQIGNMISIWVSHSIQTGNQHQAEPISNTNFLTEKAVASVTLAGNSSLCPISGWAVYSKDNSIRIGSKGDVFVIREPFISCSHLECRTFFLTQGALLNDKHSNGTVKDRSPYRTLMSCPVGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELDAPNYHYEECSCYPDAGEITCVCRDNWHGSNRPWVSFNQNLEYQIGYICSGVFGDNPRPNDGTGSCGPVSANGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVSWSWPDGAELPFTIDKYPYDVPDYA

SEQ ID NO: 33 - DNA, 875 bp, Artificial Sequence
  Influenza H1N1&H5N1 M1 consensus sequence
ggtaccggatccgccaccatggactggacctggattctgttcctggtggccgctgccacccgggtgcacagcatgagcctgctgaccgaggtggagacctacgtgctgtccatcatccccagcggccctctgaaggccgagatcgcccagcggctggaagatgtgttcgccggcaagaacaccgacctggaagccctgatggaatggctgaaaacccggcccatcctgagccccctgaccaagggcatcctgggcttcgtgttcaccctgaccgtgcccagcgagcggggcctgcagcggcggagattcgtgcagaacgccctgaacggcaacggcgaccccaacaacatggaccgggccgtgaagctgtacaagaagctgaagcgggagatcaccttccacggcgccaaagaggtggccctgagctacagcacaggcgccctggccagctgcatgggcctgatctacaaccggatgggcaccgtgaccaccgaggtggccttcggcctggtgtgcgccacctgcgagcagatcgccgacagccagcacagatcccaccggcagatggccaccaccaccaaccccctgatccggcacgagaaccggatggtcctggcctccaccaccgccaaggccatggaacagatggccggcagcagcgagcaggccgccgaagccatggaagtggccagccaggccaggcagatggtgcaggccatgcggaccatcggcacccaccccagcagcagcgccggactgcgggacgacctgctggaaaacctgcaggcctaccagaaacggatgggcgtgcagatgcagcggttcaagtacccctacgacgtgcccgactacgcctgatgagcggccgcgagctc

SEQ ID NO: 34 - protein, 279 aa, Artificial Sequence
  Influenza H1N1&H5N1 M1 consensus protein sequence
MDWTWILFLVAAATRVHSMSLLTEVETYVLSIIPSGPLKAEIAQRLEDVFAGKNTDLEALMEWLKTRPILSPLTKGILGFVFTLTVPSERGLQRRRFVQNALNGNGDPNNMDRAVKLYKKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLRDDLLENLQAYQKRMGVQMQRFKYPYDVPDYA

SEQ ID NO: 35 - DNA, 1700 bp, Artificial Sequence
  Influenza H5N1 M2E-NP consensus sequence
ggtaccgaattcgccaccatggactggacctggatcctgttcctggtcgctgccgccaccagggtgcacagcagcctgctgaccgaggtggagacccccacccggaacgagtggggctgccggtgcagcgacagcagcgaccggggcaggaagcggagaagcgccagccagggcaccaagcggagctacgagcagatggaaacaggcggcgagcggcagaacgccaccgagatccgggccagcgtgggcagaatggtcggcggcatcggccggttctacatccagatgtgcaccgagctgaagctgtccgactacgagggccggctgatccagaacagcatcaccatcgagcggatggtgctgtccgccttcgacgagcggcggaacagatacctggaagagcaccccagcgccggcaaggaccccaagaaaaccggcggacccatctaccggcggagggacggcaagtgggtgcgggagctgatcctgtacgacaaagaggaaatccggcggatctggcggcaggccaacaacggcgaggacgccacagccggcctgacccacctgatgatctggcacagcaacctgaacgacgccacctaccagcggacaagggctctggtccggaccggcatggacccccggatgtgcagcctgatgcagggcagcacactgcccagaagaagcggagccgctggcgcagccgtgaagggcgtgggcaccatggtgatggaactgatccggatgatcaagcggggcatcaacgaccggaatttttggaggggcgagaacggcaggcggacccggatcgcctacgagcggatgtgcaacatcctgaagggcaagttccagacagccgcccagcgggccatgatggaccaggtccgggagagccggaaccccggcaacgccgagatcgaggacctgatcttcctggccagaagcgccctgatcctgcggggcagcgtggcccacaagagctgcctgcccgcctgcgtgtacggactggccgtggccagcggctacgacttcgagcgggagggctacagcctggtcggcatcgaccccttccggctgctgcagaactcccaggtgttcagcctgatccggcccaacgagaaccccgcccacaagtcccagctggtctggatggcctgccacagcgccgccttcgaggatctgagagtgagcagcttcatccggggcaccagagtggtgcccaggggccagctgtccaccaggggcgtgcagatcgccagcaacgagaacatggaagccatggacagcaacaccctggaactgcggagccggtactgggccatccggaccagaagcggcggcaacaccaaccagcagcgggccagcgccggacagatcagcgtgcagcccaccttctccgtgcagcggaacctgcccttcgagagggccaccatcatggccgccttcaccggcaacaccgagggccggaccagcgacatgcggaccgagatcatcaggatgatggaaagcgccaggcccgaggacgtgagcttccagggcaggggcgtgttcgagctgtccgatgagaaggccaccaaccccatcgtgcccagcttcgacatgaacaacgagggcagctacttcttcggcgacaacgccgaggaatacgacaactacccctacgacgtgcccgactacgcctgatgagcggccgcgagctc

SEQ ID NO: 36 - protein, 554 aa, Artificial Sequence
  Influenza H5N1 M2E-NP consensus protein sequence
MDWTWILFLVAAATRVHSSLLTEVETPTRNEWGCRCSDSSDRGRKRRSASQGTKRSYEQMETGGERQNATEIRASVGRMVGGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEEYDNYPYDVPDYA

SEQ ID NO: 37 - protein, 18 aa, Artificial Sequence
  IgE leader sequence
MDWTWILFLVAAATRVHS

SEQ ID NO: 38 - DNA, 818 bp, Artificial Sequence
  HPV genotype 16 E6-E7 DNA sequence
gaattcgccaccatggactggacctggatcctgttcctggtggccgccgccacacgggtgcacagcttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctgagaggccggaagagaagaagccacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccctgatgagcggccgc

SEQ ID NO: 39 - protein, 264 aa, Artificial Sequence
  HPV genotype 16 E6-E7 protein sequence
MDWTWILFLVAAATRVHSFQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLRGRKRRSHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 40 - protein, 15 aa, Artificial Sequence
  HPV E6 immunodominant epitope
LCIVYRDGNPYAVCD

SEQ ID NO: 41 - protein, 15 aa, Artificial Sequence
  HPV E7 immunodominant epitope
AEPDRAHYNIVTFCC

SEQ ID NO: 42 - protein, 142 aa, Artificial Sequence
  HPV E6 consensus sequence
FQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQL

SEQ ID NO: 43 - protein, 97 aa, Artificial Sequence
  HPV E7 consensus sequence
HGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 44 - DNA, 1766 bp, Artificial Sequence
  HCV genotype 1a and 1b consensus E1-E2 DNA sequence
gaattcgccaccatggactggacctggatcctgttcctggtggccgctgcaacacgggtgcacagctaccaagtgaggaatagcagcggcctgtaccacgtgaccaacgactgctccaacagcagcatcgtgtacgaggccgccgacatgatcatgcacacccccggctgtgtgccctgtgtgagagagggcaacagctccagatgctgggtggccctgacccctaccgtggccgccagagatggcagcctgcccaccaccaccctgaggagacacgtggacctgcttgtgggcagcgccaccctgtgtagcgccatgtatgtgggcgatctgtgtggcagcgtgtttcttgtgggccagctgttcaccttcagccccagaaggcactggaccgtgcaggactgtaactgctccatctaccccggccacatcaccggccacagaatggcctgggacatgatgatgaactggagccctaccaccgccctggtggtgtcccagctgctgagaatccctcaggccatcgtggacatggtggccggagcccactggggcgtgctggccggcatcgcctacttcagcatggtgggcaactgggccaaggtgctcgtggtgctgctgctgttcgccggcgtggacggcagaggcaggaagagaaggagcgagacccacgtgaccggcggcaccgccggcagaaccacagccggccttgtgggcctgttcacccctggcgccaagcagaacatccagctgatcaacaccaacggcagctggcacatcaacagcaccgccctgaactgtaacgacagcctgaacaccggctggctggccggcctgttctaccagcacaagttcaacagcagcggctgccccgagagaatggccagctgtagacccctggatgagttcgcccagggctggggccccatcacctacgccaatggcagcggccctgaccagagaccctactgctggcactacgcccccagaccctgtggcatcgtgcccgccaagagcgtgtgtggccccgtgtactgcttcacccctagccccgtggttgtgggcaccaccgacagaagcggagcccccacctacagctggggcgagaacgagaccgacgtgctggtgctgaacaacaccagaccccccctgggcaattggttcggctgtacctggatgaacagcaccggcttcaccaaagtgtgtggcgcccctccctgtgtgatcggcggagtgggcaacaacaccctgacctgccccaccgactgcttcagaaagcaccccgaggccacctactccagatgtggcagcggaccttggctgacccccagatgtatggtggactacccctacaggctgtggcactacccctgtaccgtgaacttcaccatcttcaaagtgaggatgtatgtggggggcgtggagcacagactggaggccgcctgtaattggaccaggggcgagagatgtgacctggaggaccgggatagaagcgagctgtcccctctgctgctgtccaccaccgagtggcaggtgctgccttgtagcttcaccaccctgcccgccctgagcaccggcctgatccacctgcaccagaacatcgtggacgtgcagtacctgtacggagtgggctctagcatcgtgtcctgggccatcaagtgggagtacgtggtgctgctgttcctgctgctggccgacgccagagtgtgtagctgcctgtggatgatgctgctgatcagccaggccgaggcctgatgagcggccgc

SEQ ID NO: 45 - protein, 580 aa, Artificial Sequence
  HCV genotype 1a and 1b consensus E1-E2 protein sequence
MDWTWILFLVAAATRVHSYQVRNSSGLYHVTNDCSNSSIVYEAADMIMHTPGCVPCVREGNSSRCWVALTPTVAARDGSLPTTTLRRHVDLLVGSATLCSAMYVGDLCGSVFLVGQLFTFSPRRHWTVQDCNCSIYPGHITGHRMAWDMMMNWSPTTALVVSQLLRIPQAIVDMVAGAHWGVLAGIAYFSMVGNWAKVLVVLLLFAGVDGRGRKRRSETHVTGGTAGRTTAGLVGLFTPGAKQNIQLINTNGSWHINSTALNCNDSLNTGWLAGLFYQHKFNSSGCPERMASCRPLDEFAQGWGPITYANGSGPDQRPYCWHYAPRPCGIVPAKSVCGPVYCFTPSPVVVGTTDRSGAPTYSWGENETDVLVLNNTRPPLGNWFGCTWMNSTGFTKVCGAPPCVIGGVGNNTLTCPTDCFRKHPEATYSRCGSGPWLTPRCMVDYPYRLWHYPCTVNFTIFKVRMYVGGVEHRLEAACNWTRGERCDLEDRDRSELSPLLLSTTEWQVLPCSFTTLPALSTGLIHLHQNIVDVQYLYGVGSSIVSWAIKWEYVVLLFLLLADARVCSCLWMMLLISQAEA

SEQ ID NO: 46 - protein, 192 aa, Artificial Sequence
  HCV E1 consensus sequence
YQVRNSSGLYHVTNDCSNSSIVYEAADMIMHTPGCVPCVREGNSSRCWVALTPTVAARDGSLPTTTLRRHVDLLVGSATLCSAMYVGDLCGSVFLVGQLFTFSPRRHWTVQDCNCSIYPGHITGHRMAWDMMMNWSPTTALVVSQLLRIPQAIVDMVAGAHWGVLAGIAYFSMVGNWAKVLVVLLLFAGVDG

SEQ ID NO: 47 - protein, 363 aa, Artificial Sequence
  HCV E2 consensus sequence
ETHVTGGTAGRTTAGLVGLFTPGAKQNIQLINTNGSWHINSTALNCNDSLNTGWLAGLFYQHKFNSSGCPERMASCRPLDEFAQGWGPITYANGSGPDQRPYCWHYAPRPCGIVPAKSVCGPVYCFTPSPVVVGTTDRSGAPTYSWGENETDVLVLNNTRPPLGNWFGCTWMNSTGFTKVCGAPPCVIGGVGNNTLTCPTDCFRKHPEATYSRCGSGPWLTPRCMVDYPYRLWHYPCTVNFTIFKVRMYVGGVEHRLEAACNWTRGERCDLEDRDRSELSPLLLSTTEWQVLPCSFTTLPALSTGLIHLHQNIVDVQYLYGVGSSIVSWAIKWEYVVLLFLLLADARVCSCLWMMLLISQAEA

SEQ ID NO: 48 - protein, 110 aa, Artificial Sequence
  proinsulin antigen
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 49 - protein, 26 aa, Artificial Sequence
  insulin antigen 1
MRLLPLLALLASHLVEALYLVCGERG

SEQ ID NO: 50 - protein, 9 aa, Artificial Sequence
  insulin antigen 2
LYLVCGERG

SEQ ID NO: 51 - protein, 15 aa, Artificial Sequence
  insulin antigen 3
GIVEQCCTSICSLYQ

SEQ ID NO: 52 - protein, 13 aa, Artificial Sequence
  G6P antigen IGRP13-25
QHLQKDYRAYYTF

SEQ ID NO: 53 - protein, 13 aa, Artificial Sequence
  G6P antigen IGRP23-35
YTFLNFMSNVGDP

SEQ ID NO: 54 - protein, 13 aa, Artificial Sequence
  G6P antigen IGRP226-238
RVLNIDLLWSVPI

SEQ ID NO: 55 - protein, 13 aa, Artificial Sequence
  G6P antigen IGRP247-259
DWIHIDTTPFAGL

SEQ ID NO: 56 - protein, 13 aa, Artificial Sequence
  G6Pase-alpha228-240
KGLGVDLLWTLEK

SEQ ID NO: 57 - protein, 13 aa, Artificial Sequence
  G6Pase-alpha249-261
EWVHIDTTPFASL

SEQ ID NO: 58 - protein, 13 aa, Artificial Sequence
  UGRP218-230
FTLGLDLSWSISL

SEQ ID NO: 59 - protein, 13 aa, Artificial Sequence
  UGRP239-251
EWIHVDSRPFASL

SEQ ID NO: 60 - protein, 14 aa, Artificial Sequence
  MOG antigen 1
HPIRALVGDEVELP

SEQ ID NO: 61 - protein, 20 aa, Artificial Sequence
  MOG antigen 2
VGWYRPPFSRVVHLYRNGKD

SEQ ID NO: 62 - protein, 26 aa, Artificial Sequence
  MOG antigen 3
LKVEDPFYWVSPGVLVLLAVLPVLLL

SEQ ID NO: 63 - protein, 13 aa, Artificial Sequence
  ZP3 PEPTIDE 330-342
NSSSSQFQIHGPR

SEQ ID NO: 64 - protein, 8 aa, Artificial Sequence
  ZP3 PEPTIDE 335-342
QFQIHGPR

SEQ ID NO: 65 - protein, 11 aa, Artificial Sequence
  ZP3 PEPTIDE 330-340
NSSSSQFQIHG

SEQ ID NO: 66 - protein, 30 aa, Artificial Sequence
  myocarditis  antigen with amino acids 614-643
SLKLMATLFSTYASADTGDSGKGKGGKKKG

SEQ ID NO: 67 - protein, 14 aa, Artificial Sequence
  myocarditis antigen with amino acids 735-747
GQFIDSGKAGAEKL

SEQ ID NO: 68 - protein, 14 aa, Artificial Sequence
  myocarditis antigen with amino acids 947-960
DECSELKKDIDDLE

SEQ ID NO: 69 - protein, 20 aa, Artificial Sequence
  VP4  1-20
MGAQVSTQKTGAHETSLSAS

SEQ ID NO: 70 - protein, 20 aa, Artificial Sequence
  VP4 21-40
GNSIIHYTNINYYKDAASNS

SEQ ID NO: 71 - protein, 20 aa, Artificial Sequence
  VP4  31-50
NYYKDAASNSANRQDFTQDP

SEQ ID NO: 72 - protein, 20 aa, Artificial Sequence
  VP4  41-60
ANRQDFTQDPSKFTEPVKDV

SEQ ID NO: 73 - protein, 20 aa, Artificial Sequence
  VP4 51-70
SKFTEPVKDVMIKSLPALNS

SEQ ID NO: 74 - protein, 20 aa, Artificial Sequence
  VP2  61-80
MIKSLPALNSPTVEECGYSD

SEQ ID NO: 75 - protein, 20 aa, Artificial Sequence
  VP2  71-90
PTVEECGYSDRVRSITLGNS

SEQ ID NO: 76 - protein, 20 aa, Artificial Sequence
  VP2 81-100
RVRSITLGNSTITTQECANV

SEQ ID NO: 77 - protein, 20 aa, Artificial Sequence
  VP2  91-110
TITTQECANVVVGYGVWPDY

SEQ ID NO: 78 - protein, 20 aa, Artificial Sequence
  VP2  111-130
LSDEEATAEDQPTQPDVATC

SEQ ID NO: 79 - protein, 20 aa, Artificial Sequence
  VP2  121-140
QPTQPDVATCRFYTLNSVKW

SEQ ID NO: 80 - protein, 20 aa, Artificial Sequence
  VP2  131-150
RFYTLNSVKWEMQSAGWWWK

SEQ ID NO: 81 - protein, 20 aa, Artificial Sequence
  VP2  151-170
FPDALSEMGLFGQNMQYHYL

SEQ ID NO: 82 - protein, 20 aa, Artificial Sequence
  VP2  161-180
FGQNMQYHYLGRSGYTIHVQ

SEQ ID NO: 83 - protein, 20 aa, Artificial Sequence
  VP2  171-190
GRSGYTIHVQCNASKFHQGC

SEQ ID NO: 84 - protein, 20 aa, Artificial Sequence
  VP2 181-200
CNASKFHQGCLLVVCVPEAE

SEQ ID NO: 85 - protein, 20 aa, Artificial Sequence
  VP2 211-230
AYGDLCGGETAKSFEQNAAT

SEQ ID NO: 86 - protein, 20 aa, Artificial Sequence
  VP2 221-240
AKSFEQNAATGKTAVQTAVC

SEQ ID NO: 87 - protein, 20 aa, Artificial Sequence
  VP2 231-250
GKTAVQTAVCNAGMGVGVGN

SEQ ID NO: 88 - protein, 20 aa, Artificial Sequence
  VP2 251-270
LTIYPHQWINLRTNNSATIV

SEQ ID NO: 89 - protein, 20 aa, Artificial Sequence
  VP2 261-280
LRTNNSATIVMPYINSVPMD

SEQ ID NO: 90 - protein, 20 aa, Artificial Sequence
  VP2 271-290
MPYINSVPMDNMFRHNNFTL

SEQ ID NO: 91 - protein, 20 aa, Artificial Sequence
  VP2 281-300
NMFRHNNFTLMIIPFAPLDY

SEQ ID NO: 92 - protein, 20 aa, Artificial Sequence
  VP3 321-340
YNGLRLAGHQGLPTMLTPGS

SEQ ID NO: 93 - protein, 20 aa, Artificial Sequence
  VP3 351-370
SPSAMPQFDVTPEMNIPGQV

SEQ ID NO: 94 - protein, 20 aa, Artificial Sequence
  VP3 361-380
TPEMNIPGQVRNLMEIAEVD

SEQ ID NO: 95 - protein, 20 aa, Artificial Sequence
  VP3 371-390
RNLMEIAEVDSVVPINNLKA

SEQ ID NO: 96 - protein, 20 aa, Artificial Sequence
  VP3 381-400
SVVPINNLKANLMTMEAYRV

SEQ ID NO: 97 - protein, 20 aa, Artificial Sequence
  VP3 391-410
NLMTMEAYRVQVRSTDEMGG

SEQ ID NO: 98 - protein, 20 aa, Artificial Sequence
  VP3 401-420
QVRSTDEMGGQIFGFPLQPG

SEQ ID NO: 99 - protein, 20 aa, Artificial Sequence
  VP3 411-430
QIFGFPLQPGASSVLQRTLL

SEQ ID NO: 100 - protein, 20 aa, Artificial Sequence
  VP3 421-440
ASSVLQRTLLGEILNYYTHW

SEQ ID NO: 101 - protein, 20 aa, Artificial Sequence
  VP3 431-450
GEILNYYTHWSGSLKLTFVF

SEQ ID NO: 102 - protein, 20 aa, Artificial Sequence
  VP3 441-460
SGSLKLTFVFCGSAMATGKF

SEQ ID NO: 103 - protein, 20 aa, Artificial Sequence
  VP3 511-530
DDKYTASGFISCWYQTNVIV

SEQ ID NO: 104 - protein, 20 aa, Artificial Sequence
  VP3 541-560
MCFVSACNDFSVRMLRDTQF

SEQ ID NO: 105 - protein, 20 aa, Artificial Sequence
  VP1 671-690
LRRKMEMFTYIRCDMELTFV

SEQ ID NO: 106 - protein, 20 aa, Artificial Sequence
  VP1 721-740
VPTSVNDYVWQTSTNPSIFW

SEQ ID NO: 107 - protein, 20 aa, Artificial Sequence
  VP1 731-750
QTSTNPSIFWTEGNAPPRMS

SEQ ID NO: 108 - protein, 20 aa, Artificial Sequence
  VP1 741-760
TEGNAPPRMSIPFMSIGNAY

SEQ ID NO: 109 - protein, 20 aa, Artificial Sequence
  VP1 751-770
IPFMSIGNAYTMFYDGWSNF

SEQ ID NO: 110 - protein, 20 aa, Artificial Sequence
  VP1 771-790
SRDGIYGYNSLNNMGTIYAR

SEQ ID NO: 111 - protein, 20 aa, Artificial Sequence
  VP1 781-800
LNNMGTIYARHVNDSSPGGL

SEQ ID NO: 112 - protein, 20 aa, Artificial Sequence
  VP1 791-810
HVNDSSPGGLTSTIRIYFKP

SEQ ID NO: 113 - protein, 20 aa, Artificial Sequence
  VP1 831-850
SVNFDVEAVTAERASLITTG

SEQ ID NO: 114 - protein, 19 aa, Artificial Sequence
  group A streptococcal M5 protein  NT4
GLKTENEGLKTENEGLKTE

SEQ ID NO: 115 - protein, 18 aa, Artificial Sequence
  group A streptococcal M5 protein NT5
KKEHEAENDKLKQQRDTL

SEQ ID NO: 116 - protein, 18 aa, Artificial Sequence
  group A streptococcal M5 protein B1B2
VKDKIAKEQENKETIGTL

SEQ ID NO: 117 - protein, 18 aa, Artificial Sequence
  group A streptococcal M5 protein B2
TIGTLKKILDETVKDKIA

SEQ ID NO: 118 - protein, 18 aa, Artificial Sequence
  group A streptococcal M5 protein  B3A
IGTLKKILDETVKDKLAK

SEQ ID NO: 119 - protein, 16 aa, Artificial Sequence
  group A streptococcal M5 protein C3
KGLRRDLDASREAKKQ

SEQ ID NO: 120 - protein, 18 aa, Artificial Sequence
  M5 epitope protein 27-44
LKTKNEGLKTENEGLKTE

SEQ ID NO: 121 - protein, 18 aa, Artificial Sequence
  M5 epitope protein 59-76 (NT5)
KKEHEAENDKLKQQRDTL

SEQ ID NO: 122 - protein, 18 aa, Artificial Sequence
  M5 epitope protein 72-89 (NT6)
QRDTLSTQKETLEREVQN

SEQ ID NO: 123 - protein, 18 aa, Artificial Sequence
  M5 epitope protein 85-102 (NT7)
REVQNTQYNNETLKIKNG

SEQ ID NO: 124 - protein, 18 aa, Artificial Sequence
  M5 epitope protein 98-115 (NT8)
KIKNGDLTKELNKTRQEL

SEQ ID NO: 125 - protein, 19 aa, Artificial Sequence
  M5 epitope protein  111-129 (B1A)
TRQELANKQQESKENEKAL

SEQ ID NO: 126 - protein, 18 aa, Artificial Sequence
  M5 epitope protein 150-167 (B2)
TIGTLKKILDETVKDKIA

SEQ ID NO: 127 - protein, 18 aa, Artificial Sequence
  M5 epitope protein 176-193 (B3A)
IGTLKKILDETVKDKLAK

SEQ ID NO: 128 - protein, 35 aa, Artificial Sequence
  M5 epitope protein 1-35
AVTRGTINDPQRAKEALDKYELENHDLKTKNEGLK

SEQ ID NO: 129 - protein, 27 aa, Artificial Sequence
  M5 epitope protein 28-54
KTKNEGLKTENEGLKTENEGLKTENEG

SEQ ID NO: 130 - protein, 16 aa, Artificial Sequence
  M5 epitope protein 55-70
LKTEKKEHEAENDKLK

SEQ ID NO: 131 - protein, 30 aa, Artificial Sequence
  M5 epitope protein 103-132
DLTKELNKTRQELANKQQESKENEKAINEL

SEQ ID NO: 132 - protein, 30 aa, Artificial Sequence
  M5 epitope protein  133-162
LEKTVKDKIAKEQENKETIGTLKKILDETV

SEQ ID NO: 133 - protein, 16 aa, Artificial Sequence
  M5 epitope protein 209-223
TIGTLKKILDETVKDK

SEQ ID NO: 134 - protein, 21 aa, Artificial Sequence
  M5 epitope protein 217-237
ISDASRKGLRRDLDASREAKK

SEQ ID NO: 135 - protein, 20 aa, Artificial Sequence
  M5 epitope protein 300-319
DASREAKKQVEKAIEEANSK

SEQ ID NO: 136 - protein, 20 aa, Artificial Sequence
  M5 epitope protein 312-331
ALEEANSKLAALEKLNKELE

SEQ ID NO: 137 - protein, 31 aa, Artificial Sequence
  M5 epitope protein 329-359
ELEESKKLTEKEKAELQAKLEAEAKQLKEQL

SEQ ID NO: 138 - protein, 30 aa, Artificial Sequence
  M5 epitope protein 359-388
AKQAEELAKLRAGKASDSQTPDTKPGNKAV

SEQ ID NO: 139 - protein, 37 aa, Artificial Sequence
  M5 epitope protein 389-425
VPGKGQAPQAGTKPNQNKAPMKETKRQLPSTGETANP

SEQ ID NO: 140 - protein, 19 aa, Artificial Sequence
  M5 epitope protein 295-313
LRRDLDASREAKKQVEKAI

SEQ ID NO: 141 - protein, 20 aa, Artificial Sequence
  M5 epitope protein 305-324
AKKQVEKALEEANSKLAALE

SEQ ID NO: 142 - protein, 20 aa, Artificial Sequence
  M5 epitope protein 335-354
KLTEKEKAELQAKLEAEAKA

SEQ ID NO: 143 - protein, 20 aa, Artificial Sequence
  M5 epitope protein 345-364
QAKLEAEAKALKEQLAKQAE

SEQ ID NO: 144 - protein, 20 aa, Artificial Sequence
  M5 epitope protein 355-374
LKEQLAKQAEELAKLRAGKA

SEQ ID NO: 145 - protein, 25 aa, Artificial Sequence
  M5 epitope protein 1-25
TVTRGTISDPQRAKEALDKYELENH

SEQ ID NO: 146 - protein, 24 aa, Artificial Sequence
  M5 epitope protein 81-96
DKLKQQRDTLSTQKETLEREVQNI

SEQ ID NO: 147 - protein, 15 aa, Artificial Sequence
  M5 epitope protein 163-177
ETIGTLKKILDETVK

SEQ ID NO: 148 - protein, 18 aa, Artificial Sequence
  M5 epitope protein 1-18
AVTRGTINDPQRAKEALD

SEQ ID NO: 149 - protein, 18 aa, Artificial Sequence
  M5 epitope protein 14-31
KEALDKYELENHDLKTKN

SEQ ID NO: 150 - protein, 18 aa, Artificial Sequence
  M5 epitope protein 27-44
LKTKNEGLKTENEGLKTE

SEQ ID NO: 151 - protein, 19 aa, Artificial Sequence
  M5 epitope protein 40-58
GLKTENEGLKTENEGLKTE

SEQ ID NO: 152 - protein, 18 aa, Artificial Sequence
  M5 epitope protein 59-76
KKEHEAENDKLKQQRDTL

SEQ ID NO: 153 - protein, 18 aa, Artificial Sequence
  M5 epitope protein 72-89
QRDTLSTQKETLEREVQN

SEQ ID NO: 154 - protein, 18 aa, Artificial Sequence
  M5 epitope protein 85-102
REVQNTQYNNETLKIKNG

SEQ ID NO: 155 - protein, 18 aa, Artificial Sequence
  M5 epitope protein 98-115
KIKNGDLTKELNKTRQEL

SEQ ID NO: 156 - protein, 19 aa, Artificial Sequence
  M5 epitope protein 111-129
TRQELANKQQESKENEKAL

SEQ ID NO: 157 - protein, 18 aa, Artificial Sequence
  M5 epitope protein 124-141
ENEKALNELLEKTVKDKI

SEQ ID NO: 158 - protein, 18 aa, Artificial Sequence
  M5 epitope protein 137-154
VKDKIAKEQENKETIGTL

SEQ ID NO: 159 - protein, 18 aa, Artificial Sequence
  M5 epitope protein 150-167
TIGTLKKILDETVKDKIA

SEQ ID NO: 160 - protein, 18 aa, Artificial Sequence
  M5 epitope protein 163-180
KDKIAKEQENKETIGTLK

SEQ ID NO: 161 - protein, 18 aa, Artificial Sequence
  M5 epitope protein 176-193
IGTLKKILDETVKDKLAK

SEQ ID NO: 162 - protein, 18 aa, Artificial Sequence
  M5 epitope protein 189-206
DKLAKEQKSKQNIGALKQ

SEQ ID NO: 163 - protein, 18 aa, Artificial Sequence
  M5 epitope protein 202-219
GALKQELAKKDEANKISD

SEQ ID NO: 164 - protein, 18 aa, Artificial Sequence
  M5 epitope protein 215-232
NKISDASRKGLRRDLDAS

SEQ ID NO: 165 - protein, 18 aa, Artificial Sequence
  M5 epitope protein 228-245
DLDASREAKKQLEAEHQK

SEQ ID NO: 166 - protein, 18 aa, Artificial Sequence
  M5 epitope protein 241-258
AEHQKLEEQNKISEASRK

SEQ ID NO: 167 - protein, 18 aa, Artificial Sequence
  M5 epitope protein 254-271
EASRKGLRRDLDASREAK

SEQ ID NO: 168 - protein, 18 aa, Artificial Sequence
  M5 epitope protein 267-284
SREAKKQLEAEQQKLEEQ

SEQ ID NO: 169 - protein, 18 aa, Artificial Sequence
  M5 epitope protein 280-297
KLEEQNKISEASRKGLRR

SEQ ID NO: 170 - protein, 16 aa, Artificial Sequence
  M5 epitope protein 293-308
KGLRRDLDASREAKKQ

SEQ ID NO: 171 - protein, 12 aa, Artificial Sequence
  thyroglobulin antigen 1
NIFEXQVDAQPL

SEQ ID NO: 172 - protein, 22 aa, Artificial Sequence
  thyroglobulin antigen-2
YSLEHSTDDXASFSRALENATR

SEQ ID NO: 173 - protein, 20 aa, Artificial Sequence
  thyroglobulin antigen-3
RALENATRDXFIICPIIDMA

SEQ ID NO: 174 - protein, 14 aa, Artificial Sequence
  thyroglobulin antigen-4
LLSLQEPGSKTXSK

SEQ ID NO: 175 - protein, 16 aa, Artificial Sequence
  thyroglobulin antigen-5
EHSTDDXASFSRALEN

SEQ ID NO: 176 - protein, 14 aa, Artificial Sequence
  TPO antigen-1
LKKRGILSPAQLLS

SEQ ID NO: 177 - protein, 16 aa, Artificial Sequence
  TPO antigen-2
SGVIARAAEIMETSIQ

SEQ ID NO: 178 - protein, 14 aa, Artificial Sequence
  TPO antigen-3
PPVREVTRHVIQVS

SEQ ID NO: 179 - protein, 15 aa, Artificial Sequence
  TPO antigen-4
PRQQMNGLTSFLDAS

SEQ ID NO: 180 - protein, 15 aa, Artificial Sequence
  TPO antigen-5
LTALHTLWLREHNRL

SEQ ID NO: 181 - protein, 15 aa, Artificial Sequence
  TPO antigen-6
HNRLAAALKALNAHW

SEQ ID NO: 182 - protein, 14 aa, Artificial Sequence
  TPO antigen-7
ARKVVGALHQIITL

SEQ ID NO: 183 - protein, 16 aa, Artificial Sequence
  TPO antigen-7
LPGLWLHQAFFSPWTL

SEQ ID NO: 184 - protein, 17 aa, Artificial Sequence
  TPO antigen-8
MNEELTERLFVLSNSST

SEQ ID NO: 185 - protein, 11 aa, Artificial Sequence
  TPO antigen-9
LDLASINLQRG

SEQ ID NO: 186 - protein, 16 aa, Artificial Sequence
  TPO antigen-10
RSVADKILDLYKHPDN

SEQ ID NO: 187 - protein, 14 aa, Artificial Sequence
  TPO antigen-11
IDVWLGGLAENFLP

SEQ ID NO: 188 - protein, 12 aa, Artificial Sequence
  amino acids 34-44 in human pendrin
QQQHERRKQERK

SEQ ID NO: 189 - protein, 14 aa, Artificial Sequence
  amino acids 630-643 in human pendrin
PTKEIEIQVDWNSE

SEQ ID NO: 190 - protein, 20 aa, Artificial Sequence
  Human S-Antigen Peptide 19
VQHAPLEMGPQPRAEATWQF

SEQ ID NO: 191 - protein, 20 aa, Artificial Sequence
  Human S-Antigen Peptide 35
GFLGELTSSEVATEVPFRLM

SEQ ID NO: 192 - protein, 20 aa, Artificial Sequence
  Human S-Antigen Peptide 36
VATEVPFRLMHPQPEDPAKE

SEQ ID NO: 193 - protein, 20 aa, Artificial Sequence
  Human IRBP peptide
YLLTSHRTATAAEEFAFLMQ

SEQ ID NO: 194 - DNA, 3512 bp, Artificial Sequence
  hTERT nucleic acid
ggtaccgaattcgccaccatggactggacctggatcctgttcctggtggccgctgccacaagagtgcacagccccagggcccccaggtgcagagccgtgcggagcctgctgcggagccactaccgggaggtgctgcccctggccaccttcgtgcggaggctgggccctcaggggtggcggctggtgcagagaggcgaccctgccgccttcagagccctggtggcccagtgcctggtgtgcgtgccctgggacgccagacctccccctgccgcccctagcttccggcaggtgtcctgcctgaaagaactggtggcccgggtgctgcagcggctgtgcgagaggggcgccaagaacgtgctggccttcggcttcgccctgctggacggcgccagaggcggccctcccgaggccttcaccacctccgtgagaagctacctgcccaacaccgtgaccgacgccctgagaggcagcggcgcttggggcctgctgctgcgcagagtgggcgacgacgtgctggtgcacctgctggccagatgcgccctgttcgtgctggtcgcccccagctgcgcctaccaggtgtgcggcccacccctgtaccagctgggagccgccacccaggccagaccccctcctcacgcctccggccccaggcggagactgggctgcgagcgggcctggaaccacagcgtgcgggaggccggcgtgcccctgggcctgccagcccctggcgccagaagaaggggcggcagcgccagcagaagcctgcccctgcccaagcggcccagacgcggagccgcccctgagcccgagagaacccccgtgggccagggctcttgggcccaccctggccggaccagaggccccagcgaccggggcttctgcgtggtgtcccccgccagacccgccgaggaagccacctccctggaaggcgccctgagcggcaccaggcacagccaccccagcgtgggccgccagcaccacgccggaccccccagcacctccaggccccccaggccctgggacaccccttgcccccctgtgtacgccgagaccaagcacttcctgtacagcagcggcgacaaagagcagctgcggcccagcttcctgctgtccagcctgaggccctccctgaccggcgctaggcgcctggtggagaccatctttctgggcagccggccctggatgcccggcacccccaggcggctgcccaggctgccccagcggtactggcagatgaggcctctgttcctggaactgctgggcaaccacgcccagtgcccctacggcgtgctgctgaaaacccactgccccctgagagccgccgtgaccccagccgccggagtgtgcgccagagagaagcctcagggcagcgtggccgctcccgaggaagaggacaccgaccccagacgcctggtgcagctgctgcggcagcacagcagcccttggcaggtgtacggcttcgtgcgggcctgcctgagaaggctggtgccccctggcctgtggggcagcaggcacaacgagcggcggtttctgcggaacaccaagaagttcatcagcctggggaagcacgccaagctgtccctgcaggaactgacctggaagatgagcgtgcggggctgcgcctggctgagaagatcccctggcgtgggctgcgtgcctgccgccgagcaccggctgcgggaggaaatcctggccaagttcctgcactggctgatgagcgtgtacgtggtggagctgctgagatccttcttctacgtgaccgagaccaccttccagaagaactacctgttcttctaccggaagagcgtgtggagcaagctgcagagcatcggcatccggcagcacctgaagcgggtgcagctgagagagctgtccgaggccgaagtgaggcagcaccgggaggccagacctgccctgctgaccagccggctgcggttcatccccaagcccgacggcctgcggcccatcgtgaacatggactacgtggtgggcgccaggaccttccggcgggagaagcgggccgagcggctgacctcgagggtgaaggccctgttcagcgtgctgaactacgagcgggccaggcggccaggcctgctgggcgccagcgtgctgggcctggacgacatccaccgggcctggcggaccttcgtgctgagagtgcgggcccaggacccccctcccgagctgtacttcgtgaaggtggacgtgacaggcgcctacgacaccatcccccaggaccggctgaccgaggtgatcgccagcatcatcaagccccagaacacctactgcgtgcggagatacgccgtggtgcagaaggccgcccacggccacgtgcggaaggccttcaagagccacgtgagcaccctgaccgacctgcagccctacatgcggcagttcgtggcccacctgcaggaaaccagccccctgcgggatgccgtggtgatcgagcagagcagcagcctgaacgaggccagcagcggcctgttcgacgtgttcctgagattcatgtgccaccacgccgtgcggatccggggcaagagctacgtgcagtgccagggcatcccacagggcagcatcctgtccaccctgctgtgctccctgtgctacggcgacatggaaaacaagctgttcgccggcatcaggcgggacggactgctgctgagactggtggacgacttcctgctggtgaccccccacctgacccacgccaagacctttctgcggaccctggtgcgcggcgtgcccgagtacggctgcgtggtgaacctgagaaagaccgtggtgaacttccccgtggaggacgaggccctgggcggcacagccttcgtgcagatgcctgcccatggactgttcccttggtgcgggctgctgctggacacccggaccctggaagtgcagagcgactacagcagctacgcccggaccagcatccgggcctccctgaccttcaacaggggcttcaaggccggcaggaacatgcggcggaagctgtttggcgtgctgcggctgaagtgccacagcctgtttctgtacctgcaggtgaacagcctgcagaccgtgtgcaccaacatctacaagatcctgctgctgcaggcctaccggttccacgcctgcgtgctgcagctgccctttcaccagcaggtgtggaagaaccctaccttcttcctgcgggtgatcagcgacaccgccagcctgtgctacagcatcctgaaggccaagaacgccggcatgagcctgggcgccaagggagccgccggacctctgcccagcgaggccgtgcagtggctgtgccaccaggcctttctgctgaagctgacccggcaccgggtgacctacgtgcccctgctgggcagcctgcggaccgcccagacccagctgtcccggaagctgcctggcaccaccctgacagccctggaagccgccgccaaccccgccctgccctccgacttcaagaccatcctggactacccctacgacgtgcccgactacgcctgatgagcggccgcgagctc

SEQ ID NO: 195 - protein, 1158 aa, Artificial Sequence
  hTERT amino acid sequence
MDWTWILFLVAAATRVHSPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRGCAWLRRSPGVGCVPAAEHRLREEILAKFLHWLMSVYVVELLRSFFYVTETTFQKNYLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLYLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILDYPYDVPDYA

SEQ ID NO: 196 - DNA, 789 bp, Artificial Sequence
  PSA antigen 1 nucleic acid
atgtgggtcctggtggtgttcctgactctgagcgtcacatggatcggcgccgctccactgattctgagccgcctggtgggcgggtgggagtgcgaaaagcactcccagccatggcaggtgctggtcgcttctaggggccgagcagtgtgcggaggcgtgctggtccaccctcagtgggtcctgaccgcagcccattgtatccgacagaagagcgtgattctgctggggcgacaccagccattctaccccgaggacacaggacaggtgttccaggtctctcacagttttccccatcctctgtacaacatgagcctgctgaaaaacagatatctgggacctggcgacgatagctcccatgatctgatgctgctgaggctgtccgagccagccgaactgactgacgctgtgcaggtcctggatctgcccacccaggagcctgccctgggaaccacatgttatgcttcaggctgggggagcatcgaaccagaggaacatctgactcccaagaaactgcagtgcgtggacctgcacctgattagtaacgatgtgtgtgcacaggtccattcacagaaggtgacaaagttcatgctgtgcgccggctcttggatgggcggcaagtcaacttgcagcggggactccggcgggccactggtgtgtgatggagtcctgcagggcatcacctcttggggcagtcagccttgtgccctgcctcggagaccaagtctgtacactaaggtggtccggtataggaaatggattcaggacactattgccgctaacccctgataa

SEQ ID NO: 197 - protein, 261 aa, Artificial Sequence
  PSA antigen 1 amino acid
MWVLVVFLTLSVTWIGAAPLILSRLVGGWECEKHSQPWQVLVASRGRAVCGGVLVHPQWVLTAAHCIRQKSVILLGRHQPFYPEDTGQVFQVSHSFPHPLYNMSLLKNRYLGPGDDSSHDLMLLRLSEPAELTDAVQVLDLPTQEPALGTTCYASGWGSIEPEEHLTPKKLQCVDLHLISNDVCAQVHSQKVTKFMLCAGSWMGGKSTCSGDSGGPLVCDGVLQGITSWGSQPCALPRRPSLYTKVVRYRKWIQDTIAANP

SEQ ID NO: 198 - DNA, 840 bp, Artificial Sequence
  PSA antigen 2 nucliec acid
atggactggacatggattctgttcctggtcgccgccgcaactcgcgtgcattcctgggtcctggtggtgttcctgactctgagcgtcacatggatcggcgccgctccactgattctgagccgcctggtgggcgggtgggagtgcgaaaagcactcccagccatggcaggtgctggtcgcttctaggggccgagcagtgtgcggaggcgtgctggtccaccctcagtgggtcctgaccgcagcccattgtatccgacagaagagcgtgattctgctggggcgacaccagccattctaccccgaggacacaggacaggtgttccaggtctctcacagttttccccatcctctgtacaacatgagcctgctgaaaaacagatatctgggacctggcgacgatagctcccatgatctgatgctgctgaggctgtccgagccagccgaactgactgacgctgtgcaggtcctggatctgcccacccaggagcctgccctgggaaccacatgttatgcttcaggctgggggagcatcgaaccagaggaacatctgactcccaagaaactgcagtgcgtggacctgcacctgattagtaacgatgtgtgtgcacaggtccattcacagaaggtgacaaagttcatgctgtgcgccggctcttggatgggcggcaagtcaacttgcagcggggactccggcgggccactggtgtgtgatggagtcctgcagggcatcacctcttggggcagtcagccttgtgccctgcctcggagaccaagtctgtacactaaggtggtccggtataggaaatggattcaggacactattgccgctaacccctgataa

SEQ ID NO: 199 - protein, 278 aa, Artificial Sequence
  PSA antigen 2 amino acid
MDWTWILFLVAAATRVHSWVLVVFLTLSVTWIGAAPLILSRLVGGWECEKHSQPWQVLVASRGRAVCGGVLVHPQWVLTAAHCIRQKSVILLGRHQPFYPEDTGQVFQVSHSFPHPLYNMSLLKNRYLGPGDDSSHDLMLLRLSEPAELTDAVQVLDLPTQEPALGTTCYASGWGSIEPEEHLTPKKLQCVDLHLISNDVCAQVHSQKVTKFMLCAGSWMGGKSTCSGDSGGPLVCDGVLQGITSWGSQPCALPRRPSLYTKVVRYRKWIQDTIAANP

SEQ ID NO: 200 - DNA, 2283 bp, Artificial Sequence
  PSMA antigen 1 nucleic acid
atgtggaacgcactgcatgagactgattctgctgtcgcactgggacggagaccccggtggctgtgcgctggagcactggtgctggccggcgggggattcctgctgggattcctgtttggctggtttatcaaaagctccagcgaggctaccaatattacccctaagcacaataagaaagcattcctggatgaactgaaagccgagaacatcaagaaattcctgtacaacttcacaagaattccacatctggctggcactgagcagaacttccagctggcaaaacagatccagagtcagtggaaggaatttgggctggactcagtggagctgacccactacgatgtcctgctgtcctatccaaataagactcatcccaactacatctctatcattaacgaagacggaaatgagattttcaacacctctctgtttgaaccccctccacccggctatgagaatgtcagtgacgtggtccctccattctcagccttcagcccccaggggatgcctgagggagatctggtgtacgtcaattatgctagaacagaagacttctttaagctggagagggatatgaaaatcaactgttccggcaagatcgtgattgcccggtacgggaaggtgttcagaggaaataaggtcaaaaacgctcagctggccggagctaccggcgtgatcctgtacagcgaccccgctgattattttgcacctggcgtgaagtcctatccagacggatggaatctgcccggcgggggagtgcagaggggaaacatcctgaacctgaatggagccggcgatcctctgactccaggataccccgccaacgaatacgcttatcgccggggaattgcagaggccgtgggcctgcctagcatcccagtccatcccattggctattacgatgcccagaagctgctggagaaaatgggcgggagcgctccccctgactctagttggaagggctccctgaaagtgccttacaatgtcgggccaggattcactgggaacttttctacccagaaggtgaaaatgcacatccatagtaccagcgaggtgacacgaatctacaacgtcattggcaccctgagaggcgccgtggagcctgatcgctatgtcattctgggaggccacagagactcatgggtgttcgggggaatcgatccacagagcggagcagctgtggtccatgaaattgtgcgcagctttgggaccctgaagaaagagggatggcgacccaggcgcacaatcctgttcgcatcctgggacgccgaggaatttgggctgctgggcagcacagaatgggccgaggaaaattctcgcctgctgcaggagcgaggggtggcttacatcaatgcagactcaagcattgaaggaaactataccctgcgggtggattgcacacccctgatgtacagtctggtctataacctgacaaaggagctgaaatcacctgacgagggcttcgaagggaaaagcctgtacgaatcctggactgagaagagcccatcccccgaattcagcggcatgcctaggatctctaagctgggcagtgggaacgattttgaggtgttctttcagcgcctgggaattgcctctggccgagctcggtacacaaaaaattgggagactaacaagttctcctcttacccactgtatcacagcgtgtacgagacttatgaactggtcgagaaattctacgaccccacttttaagtatcatctgaccgtggcacaggtcaggggcgggatggtgttcgaactggccaatagcatcgtcctgccatttgactgtcgagattacgctgtggtcctgcggaagtacgcagacaagatctataacatctccatgaagcacccccaggagatgaaggcctattctgtgagtttcgattccctgttttctgccgtcaaaaatttcaccgaaatcgctagtaagttttcagagcgcctgcaggacctggataagtccaatcccatcctgctgcggattatgaacgatcagctgatgttcctggaaagagcctttatcgaccctctgggcctgcctgatagaccattctacaggcacgtgatctacgcacctagttcacataacaagtacgccggcgagtctttcccagggatctatgacgctctgtttgatattgaatcaaaggtggaccccagcaaagcatggggcgaggtcaagagacagatcagcattgcagcctttacagtgcaggccgccgccgaaaccctgtccgaagtcgcttacccatacgatgtccccgattacgcatgataa

SEQ ID NO: 201 - protein, 750 aa, Artificial Sequence
  PSMA antigen 1 amino acid
MWNALHETDSAVALGRRPRWLCAGALVLAGGGFLLGFLFGWFIKSSSEATNITPKHNKKAFLDELKAENIKKFLYNFTRIPHLAGTEQNFQLAKQIQSQWKEFGLDSVELTHYDVLLSYPNKTHPNYISIINEDGNEIFNTSLFEPPPPGYENVSDVVPPFSAFSPQGMPEGDLVYVNYARTEDFFKLERDMKINCSGKIVIARYGKVFRGNKVKNAQLAGATGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWKGSLKVPYNVGPGFTGNFSTQKVKMHIHSTSEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELKSPDEGFEGKSLYESWTEKSPSPEFSGMPRISKLGSGNDFEVFFQRLGIASGRARYTKNWETNKFSSYPLYHSVYETYELVEKFYDPTFKYHLTVAQVRGGMVFELANSIVLPFDCRDYAVVLRKYADKIYNISMKHPQEMKAYSVSFDSLFSAVKNFTEIASKFSERLQDLDKSNPILLRIMNDQLMFLERAFIDPLGLPDRPFYRHVIYAPSSHNKYAGESFPGIYDALFDIESKVDPSKAWGEVKRQISIAAFTVQAAAETLSEVA

SEQ ID NO: 202 - DNA, 2334 bp, Artificial Sequence
  PSMA antigen 2 nucleic acid
atggactggacatggattctgttcctggtcgccgccgcaactcgcgtgcattcctggaacgcactgcatgagactgattctgctgtcgcactgggacggagaccccggtggctgtgcgctggagcactggtgctggccggcgggggattcctgctgggattcctgtttggctggtttatcaaaagctccagcgaggctaccaatattacccctaagcacaataagaaagcattcctggatgaactgaaagccgagaacatcaagaaattcctgtacaacttcacaagaattccacatctggctggcactgagcagaacttccagctggcaaaacagatccagagtcagtggaaggaatttgggctggactcagtggagctgacccactacgatgtcctgctgtcctatccaaataagactcatcccaactacatctctatcattaacgaagacggaaatgagattttcaacacctctctgtttgaaccccctccacccggctatgagaatgtcagtgacgtggtccctccattctcagccttcagcccccaggggatgcctgagggagatctggtgtacgtcaattatgctagaacagaagacttctttaagctggagagggatatgaaaatcaactgttccggcaagatcgtgattgcccggtacgggaaggtgttcagaggaaataaggtcaaaaacgctcagctggccggagctaccggcgtgatcctgtacagcgaccccgctgattattttgcacctggcgtgaagtcctatccagacggatggaatctgcccggcgggggagtgcagaggggaaacatcctgaacctgaatggagccggcgatcctctgactccaggataccccgccaacgaatacgcttatcgccggggaattgcagaggccgtgggcctgcctagcatcccagtccatcccattggctattacgatgcccagaagctgctggagaaaatgggcgggagcgctccccctgactctagttggaagggctccctgaaagtgccttacaatgtcgggccaggattcactgggaacttttctacccagaaggtgaaaatgcacatccatagtaccagcgaggtgacacgaatctacaacgtcattggcaccctgagaggcgccgtggagcctgatcgctatgtcattctgggaggccacagagactcatgggtgttcgggggaatcgatccacagagcggagcagctgtggtccatgaaattgtgcgcagctttgggaccctgaagaaagagggatggcgacccaggcgcacaatcctgttcgcatcctgggacgccgaggaatttgggctgctgggcagcacagaatgggccgaggaaaattctcgcctgctgcaggagcgaggggtggcttacatcaatgcagactcaagcattgaaggaaactataccctgcgggtggattgcacacccctgatgtacagtctggtctataacctgacaaaggagctgaaatcacctgacgagggcttcgaagggaaaagcctgtacgaatcctggactgagaagagcccatcccccgaattcagcggcatgcctaggatctctaagctgggcagtgggaacgattttgaggtgttctttcagcgcctgggaattgcctctggccgagctcggtacacaaaaaattgggagactaacaagttctcctcttacccactgtatcacagcgtgtacgagacttatgaactggtcgagaaattctacgaccccacttttaagtatcatctgaccgtggcacaggtcaggggcgggatggtgttcgaactggccaatagcatcgtcctgccatttgactgtcgagattacgctgtggtcctgcggaagtacgcagacaagatctataacatctccatgaagcacccccaggagatgaaggcctattctgtgagtttcgattccctgttttctgccgtcaaaaatttcaccgaaatcgctagtaagttttcagagcgcctgcaggacctggataagtccaatcccatcctgctgcggattatgaacgatcagctgatgttcctggaaagagcctttatcgaccctctgggcctgcctgatagaccattctacaggcacgtgatctacgcacctagttcacataacaagtacgccggcgagtctttcccagggatctatgacgctctgtttgatattgaatcaaaggtggaccccagcaaagcatggggcgaggtcaagagacagatcagcattgcagcctttacagtgcaggccgccgccgaaaccctgtccgaagtcgcttacccatacgatgtccccgattacgcatgataa

SEQ ID NO: 203 - protein, 766 aa, Artificial Sequence
  PSMA antigen 2 amino acid
MWTWILFLVAAATRVHSWNALHETDSAVALGRRPRWLCAGALVLAGGGFLLGFLFGWFIKSSSEATNITPKHNKKAFLDELKAENIKKFLYNFTRIPHLAGTEQNFQLAKQIQSQWKEFGLDSVELTHYDVLLSYPNKTHPNYISIINEDGNEIFNTSLFEPPPPGYENVSDVVPPFSAFSPQGMPEGDLVYVNYARTEDFFKLERDMKINCSGKIVIARYGKVFRGNKVKNAQLAGATGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWKGSLKVPYNVGPGFTGNFSTQKVKMHIHSTSEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELKSPDEGFEGKSLYESWTEKSPSPEFSGMPRISKLGSGNDFEVFFQRLGIASGRARYTKNWETNKFSSYPLYHSVYETYELVEKFYDPTFKYHLTVAQVRGGMVFELANSIVLPFDCRDYAVVLRKYADKIYNISMKHPQEMKAYSVSFDSLFSAVKNFTEIASKFSERLQDLDKSNPILLRIMNDQLMFLERAFIDPLGLPDRPFYRHVIYAPSSHNKYAGESFPGIYDALFDIESKVDPSKAWGEVKRQISIAAFTVQAAAETLSEVA

SEQ ID NO: 204 - DNA, 1023 bp, Artificial Sequence
  STEAP antigen 1 nucleic acid
atggagagccgcaaggacatcacaaatcaggaagagctgtggaagatgaaaccacggagaaacctggaggaagacgattacctgcacaaggacaccggcgaaacaagtatgctgaaaagaccagtgctgctgcacctgcatcagactgctcatgcagacgagtttgattgcccctctgaactgcagcacacccaggagctgttcccacagtggcatctgcccatcaagattgccgctatcattgcttcactgacatttctgtatactctgctgagagaagtgatccaccctctggccaccagccatcagcagtacttctataagatccctattctggtcatcaacaaggtcctgccaatggtgagcatcacactgctggccctggtctacctgcctggcgtgatcgcagccattgtccagctgcacaacggaacaaagtacaagaagttcccacattggctggataagtggatgctgactaggaaacagttcgggctgctgtccttctttttcgccgtgctgcacgctatctacagcctgtcctatcccatgaggcgctcttaccgatataagctgctgaactgggcttaccagcaggtgcagcagaacaaggaggacgcatggattgaacacgatgtgtggcggatggaaatctatgtgtctctgggcattgtcgggctggccatcctggctctgctggcagtgaccagtatcccttctgtcagtgactcactgacatggcgcgagtttcactacattcagagcaagctgggaatcgtgtccctgctgctgggcaccatccatgcactgatttttgcctggaataagtggatcgatatcaagcagttcgtgtggtatactccccctacctttatgattgccgtcttcctgcccatcgtggtcctgatttttaagtccatcctgttcctgccttgtctgcgaaagaaaatcctgaaaatccgacatgggtgggaagacgtgacaaaaatcaataagaccgaaatctcaagccagctgtgataa

SEQ ID NO: 205 - protein, 339 aa, Artificial Sequence
  STEAP antigen 1 amino acid
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEISSQL

SEQ ID NO: 206 - DNA, 1074 bp, Artificial Sequence
  STEAP antigen 2 nucleic acid
atggactggacatggattctgtttctggtcgctgccgcaacccgcgtgcattcagagagccgcaaggacatcacaaatcaggaagagctgtggaagatgaaaccacggagaaacctggaggaagacgattacctgcacaaggacaccggcgaaacaagtatgctgaaaagaccagtgctgctgcacctgcatcagactgctcatgcagacgagtttgattgcccctctgaactgcagcacacccaggagctgttcccacagtggcatctgcccatcaagattgccgctatcattgcttcactgacatttctgtatactctgctgagagaagtgatccaccctctggccaccagccatcagcagtacttctataagatccctattctggtcatcaacaaggtcctgccaatggtgagcatcacactgctggccctggtctacctgcctggcgtgatcgcagccattgtccagctgcacaacggaacaaagtacaagaagttcccacattggctggataagtggatgctgactaggaaacagttcgggctgctgtccttctttttcgccgtgctgcacgctatctacagcctgtcctatcccatgaggcgctcttaccgatataagctgctgaactgggcttaccagcaggtgcagcagaacaaggaggacgcatggattgaacacgatgtgtggcggatggaaatctatgtgtctctgggcattgtcgggctggccatcctggctctgctggcagtgaccagtatcccttctgtcagtgactcactgacatggcgcgagtttcactacattcagagcaagctgggaatcgtgtccctgctgctgggcaccatccatgcactgatttttgcctggaataagtggatcgatatcaagcagttcgtgtggtatactccccctacctttatgattgccgtcttcctgcccatcgtggtcctgatttttaagtccatcctgttcctgccttgtctgcgaaagaaaatcctgaaaatccgacatgggtgggaagacgtgacaaaaatcaataagaccgaaatctcaagccagctgtgataa

SEQ ID NO: 207 - protein, 356 aa, Artificial Sequence
  STEAP antigen 2 amino acid
MDWTWILFLVAAATRVHSESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEISSQL

SEQ ID NO: 208 - DNA, 399 bp, Artificial Sequence
  PSCA antigen  nucleic acid
atggactggacatggattctgtttctggtcgccgccgcaacccgcgtgcattctgctggcctggcactgcagcctggaaccgccctgctgtgctactcttgtaaggcccaggtgagtaacgaggactgcctgcaggtcgaaaattgtactcagctgggagagcagtgctggaccgcacggatcagagcagtgggactgctgacagtcattagcaaagggtgctccctgaactgtgtggacgatagccaggattactatgtcggaaagaaaaacatcacctgctgtgacacagatctgtgtaatgcttctggcgcccacgctctgcagcccgcagccgctattctggctctgctgcccgctctgggactgctgctgtggggacccggacagctgtgataa

SEQ ID NO: 209 - protein, 131 aa, Artificial Sequence
  PSCA antigen amino acid
MDWTWILFLVAAATRVHSAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 210 - DNA, 2916 bp, Artificial Sequence
  consensus VP1-VP4 subtype A and consensus C3 amino acid
atgctgaacggcgactggaaggccaaggtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcaccggcctgttcggcgccctgctggccgacaagaagaccgaggagaccaccctgctggaggaccgcatcctgaccacccgcaacggccacaccaccagcaccacccagagcagcgtgggcgtgacctacggctacagcaccaccgaggaccacgtggccggccccaacaccagcggcctggagacccgcgtggtgcaggccgagcgcttcttcaagaagttcctgttcgactggaccaccgacaagcccttcggccacctgcacaagctggagctgcccaccgaccaccacggcgtgttcggccacctggtggacagctacgcctacatgcgcaacggctgggacgtggaggtgagcgccgtgggcaaccagttcaacggcggctgcctgctggtggccatggtgcccgagtggaaggagttcgacacccgcgagaagtaccagctgaccctgttcccccaccagttcatcagcccccgcaccaacatgaccgcccacatcaccgtgccctacctgggcgtgaaccgctacgaccagtacaagaagcacaagccctggaccctggtggtgatggtggtgagccccctgaccgtgaacaccgccgcccagatcaaggtgtacgccaacatcgcccccacctacgtgcacgtggccggcgagctgcccagcaaggagggcatcttccccgtggcctgcgccgacggctacggcggcctggtgaccaccgaccccaagaccgccgaccccgcctacggcaaggtgtacaaccccccccgcaccaactaccccggccgcttcaccaacctgctggacgtggccgaggcctgccccaccttcctgtgcttcgacgacggcaagccctacgtgaccacccgcaccgacgagacccgcctgctggccaagttcgacgtgagcctggccgccaagcacatgagcaacacctacctgagcggcatcgcccagtactacacccagtacagcggcaccatcaacctgcacttcatgttcaccggcagcaccgacagcaaggcccgctacatggtggcctacatcccccccggcgtggagaccccccccgacacccccgagcgcgccgcccactgcatccacgccgagtgggacaccggcctgaacagcaagttcaccttcagcatcccctacgtgagcgccgccgactacgcctacaccgccagcgacaccgccgagaccaccaacgtgcagggctgggtgtgcgtgtaccagatcacccacggcaaggccgagaacgacaccctggtggtgagcgtgagcgccggcaaggacttcgagctgcgcctgcccatcgacccccgccagcagaccaccgccaccggcgagagcgccgaccccgtgaccaccaccgtggagaactacggcggcgagacccaggtgcagcgccgccaccacaccgacgtgggcttcatcatggaccgcttcgtgaagatcaacagccccaagcccacccacgtgatcgacctgatgcagacccaccagcacggcctggtgggcgccctgctgcgcgccgccacctactacttcagcgacctggagatcgtggtgcgccacgacggcctgacctgggtgcccaacggcgcccccgagagcgccctgagcaacaccagcaaccccaccgcctacaacaaggcccccttcacccgcctggccctgccctacaccgccccccaccgcgtgctggccaccgtgtacaacggcaccagcaagtacaccgtgagcggcagcagccgccgcggcgacctgggcagcctggccgcccgcgtggccaagcagctgcccgccagcttcaactacggcgccatcaaggccgacaccatccacgagctgctggtgcgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 211 - protein, 971 aa, Artificial Sequence
  consensus VP1-VP4 subtype A and consensus C3 amino acid
MLNGDWKAKVQRKLKGAGQSSPATGSQNQSGNTGSIINNYYMQQYQNSMDTQLGDNAISGGSNEGSTDTTSTHTTNTQNNDWFSKLASSAFTGLFGALLADKKTEETTLLEDRILTTRNGHTTSTTQSSVGVTYGYSTTEDHVAGPNTSGLETRVVQAERFFKKFLFDWTTDKPFGHLHKLELPTDHHGVFGHLVDSYAYMRNGWDVEVSAVGNQFNGGCLLVAMVPEWKEFDTREKYQLTLFPHQFISPRTNMTAHITVPYLGVNRYDQYKKHKPWTLVVMVVSPLTVNTAAQIKVYANIAPTYVHVAGELPSKEGIFPVACADGYGGLVTTDPKTADPAYGKVYNPPRTNYPGRFTNLLDVAEACPTFLCFDDGKPYVTTRTDETRLLAKFDVSLAAKHMSNTYLSGIAQYYTQYSGTINLHFMFTGSTDSKARYMVAYIPPGVETPPDTPERAAHCIHAEWDTGLNSKFTFSIPYVSAADYAYTASDTAETTNVQGWVCVYQITHGKAENDTLVVSVSAGKDFELRLPIDPRQQTTATGESADPVTTTVENYGGETQVQRRHHTDVGFIMDRFVKINSPKPTHVIDLMQTHQHGLVGALLRAATYYFSDLEIVVRHDGLTWXPNGAPESALSNTSNPTAYNKAPFTRLALPYTAPHRVLATVYNGTSKYTVSGSSRRGDLGSLAARVAKQLPASFNYGAIKADTIHELLVRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 212 - DNA, 2916 bp, Artificial Sequence
  consensus VP1-VP4 subtype Asia 1 and consensus C3 nucleic acid
atgctgaacggcgagtggaaggccaaggtgcagaagcgcctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagccgcctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccgtggccgaggacgccgtgagcggccccaacaccagcggcctggagacccgcgtgcagcaggccgagcgcttcttcaagaagcacctgttcgactggacccccaacctggccttcggccactgccactacctggagctgcccaccgagcacaagggcgtgtacggcagcctgatggacagctacgcctacatgcgcaacggctgggacatcgaggtgaccgccgtgggcaaccagttcaacggcggctgcctgctggtggccctggtgcccgagctgaagagcctggacacccgccagaagtaccagctgaccctgttcccccaccagttcatcaacccccgcaccaacatgaccgcccacatcaacgtgcccttcgtgggcgtgaaccgctacgaccagtacgccctgcacaagccctggaccctggtggtgatggtggtggcccccctgaccgtgaagaccggcggcagcgagcagatcaaggtgtacatgaacgccgcccccacctacgtgcacgtggccggcgagctgcccagcaaggagggcatcgtgcccgtggcctgcgccgacggctacggcaacatggtgaccaccgaccccaagaccgccgaccccgtgtacggcaaggtgttcaaccccccccgcaccaacctgcccggccgcttcaccaacttcctggacgtggccgaggcctgccccaccttcctgcgcttcggcgaggtgcccttcgtgaagaccgtgaacagcggcgaccgcctgctggccaagttcgacgtgagcctggccgccggccacatgagcaacacctacctggccggcctggcccagtactacacccagtacagcggcaccatgaacgtgcacttcatgttcaccggccccaccgacgccaaggcccgctacatggtggcctacatcccccccggcatgaccccccccaccgaccccgagcgcgccgcccactgcatccacagcgagtgggacaccggcctgaacagcaagttcaccttcagcatcccctacctgagcgccgccgactacgcctacaccgccagcgacaccgccgagaccaccagcgtgcagggctgggtgtgcatctaccagatcacccacggcaaggccgagggcgacgccctggtggtgagcgtgagcgccggcaaggacttcgagttccgcctgcccgtggacgcccgccgccagaccaccaccaccggcgagagcgccgaccccgtgaccaccaccgtggagaactacggcggcgagacccagaccgcccgccgcctgcacaccgacgtggccttcgtgctggaccgcttcgtgaagctgaccgcccccaagaacacccagaccctggacctgatgcagatccccagccacaccctggtgggcgccctgctgcgcagcgccacctactacttcagcgacctggaggtggccctggtgcacaccggccccgtgacctgggtgcccaacggcagccccaaggacgccctggacaaccagaccaaccccaccgcctaccagaagcagcccatcacccgcctggccctgccctacaccgccccccaccgcgtgctggccaccgtgtacaacggcaagaccacctacggcgagacccccagccgccgcggcgacatggccgccctggcccagcgcctgagcgagcgcctgcccaccagcttcaactacggcgccgtgaaggccgagaccatcaccgagctgctgatccgcatgaagcgcgccgagacctactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 213 - protein, 971 aa, Artificial Sequence
  consensus VP1-VP4 subtype Asia 1 and consensus C3 amino acid
MLNGEWKAKVQKRLKGAGQSSPATGSQNQSGNTGSIINNYYMQQYQNSMDTQLGDNAISGGSNEGSTDTTSTHTNNTQNNDWFSRLASSAFSGLFGALLADKKTEETTLLEDRILTTRNGHTTSTTQSSVGVTYGYAVAEDAVSGPNTSGLETRVQQAERFFKKHLFDWTPNLAFGHCHYLELPTEHKGVYGSLMDSYAYMRNGWDIEVTAVGNQFNGGCLLVALVPELKSLDTRQKYQLTLFPHQFINPRTNMTAHINVPFVGVNRYDQYALHKPWTLVVMVVAPLTVKTGGSEQIKVYMNAAPTYVHVAGELPSKEGIVPVACADGYGNMVTTDPKTADPVYGKVFNPPRTNLPGRFTNFLDVAEACPTFLRFGEVPFVKTVNSGDRLLAKFDVSLAAGHMSNTYLAGLAQYYTQYSGTMNVHFMFTGPTDAKARYMVAYIPPGMTPPTDPERAAHCIHSEWDTGLNSKFTFSIPYLSAADYAYTASDTAETTSVQGWVCIYQITHGKAEGDALVVSVSAGKDFEFRLPVDARRQTTTTGESADPVTTTVENYGGETQTARRLHTDVAFVLDRFVKLTAPKNTQTLDLMQIPSHTLVGALLRSATYYFSDLEVALVHTGPVTWVPNGSPKDALDNQTNPTAYQKQPITRLALPYTAPHRVLATVYNGKTTYGETPSRRGDMAALAQRLSERLPTSFNYGAVKAETITELLIRMKRAETYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 214 - DNA, 2913 bp, Artificial Sequence
  consensus VP1-VP4 subtype C and consensus C3 nucleic acid
atgctgaacgagggctggaaggccagcgtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgaccttcggctacgccaccgccgaggacagcaccagcggccccaacaccagcggcctggagacccgcgtgcaccaggccgagcgcttcttcaagatggccctgttcgactgggtgcccagccagaacttcggccacatgcacaaggtggtgctgccccacgagcccaagggcgtgtacggcggcctggtgaagagctacgcctacatgcgcaacggctgggacgtggaggtgaccgccgtgggcaaccagttcaacggcggctgcctgctggtggccctggtgcccgagatgggcgacatcagcgaccgcgagaagtaccagctgaccctgtacccccaccagttcatcaacccccgcaccaacatgaccgcccacatcaccgtgccctacgtgggcgtgaaccgctacgaccagtacaagcagcaccgcccctggaccctggtggtgatggtggtggcccccctgaccaccaacaccgccggcgcccagcagatcaaggtgtacgccaacatcgcccccaccaacgtgcacgtggccggcgagctgcccagcaaggagggcatcttccccgtggcctgcagcgacggctacggcaacatggtgaccaccgaccccaagaccgccgaccccgtgtacggcaaggtgtacaaccccccccgcaccgccctgcccggccgcttcaccaactacctggacgtggccgaggcctgccccaccttcctgatgttcgagaacgtgccctacgtgagcacccgcaccgacggccagcgcctgctggccaagttcgacgtgagcctggccgccaagcacatgagcaacacctacctggccggcctggcccagtactacacccagtacaccggcaccatcaacctgcacttcatgttcaccggccccaccgacgccaaggcccgctacatggtggcctacgtgccccccggcatggacgcccccgacaaccccgaggaggccgcccactgcatccacgccgagtgggacaccggcctgaacagcaagttcaccttcagcatcccctacatcagcgccgccgactacgcctacaccgccagccacaaggccgagaccacctgcgtgcagggctgggtgtgcgtgtaccagatcacccacggcaaggccgacgccgacgccctggtggtgagcgccagcgccggcaaggacttcgagctgcgcctgcccgtggacgcccgcaagcagaccaccaccaccggcgagagcgccgaccccgtgaccaccaccgtggagaactacggcggcgagacccaggtgcagcgccgccaccacaccgacgtggccttcgtgctggaccgcttcgtggaggtgaccgtgagcggccgcaaccagcacaccctggacgtgatgcaggcccacaaggacaacatcgtgggcgccctgctgcgcgccgccacctactacttcagcgacctggagatcgccgtgacccacaccggcaagctgacctgggtgcccaacggcgcccccgtgagcgccctgaacaacaccaccaaccccaccgcctaccacaagggccccgtgacccgcctggccctgccctacaccgccccccaccgcgtgctggccaccgcctacaccggcaccaccacctacaccgccagcgcccgcggcgacctggcccacctgaccaccacccacgcccgccacctgcccaccagcttcaacttcggcgccgtgaaggccgagaccatcaccgagctgctggtgcgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 215 - protein, 970 aa, Artificial Sequence
  consensus VP1-VP4 subtype C and consensus C3 amino acid
MLNEGWKASVQRKLKGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTFGYATAEDSTSGPNTSGLETRVHQAERFFKMALFDWVPSQNFGHMHKVVLPHEPKGVYGGLVKSYAYMRNGWDVEVTAVGNQFNGGCLLVALVPEMGDISDREKYQLTLYPHQFINPRTNMTAHITVPYVGVNRYDQYKQHRPWTLVVMVVAPLTTNTAGAQQIKVYANIAPTNVHVAGELPSKEGIFPVACSDGYGNMVTTDPKTADPVYGKVYNPPRTALPGRFTNYLDVAEACPTFLMFENVPYVSTRTDGQRLLAKFDVSLAAKHMSNTYLAGLAQYYTQYTGTINLHFMFTGPTDAKARYMVAYVPPGMDAPDNPEEAAHCIHAEWDTGLNSKFTFSIPYISAADYAYTASHKAETTCVQGWVCVYQITHGKADADALVVSASAGKDFELRLPVDARKQTTTTGESADPVTTTVENYGGETQVQRRHHTDVAFVLDRFVEVTVSGRNQHTLDVMQAHKDNIVGALLRAATYYFSDLEIAVTHTGKLTWVPNGAPVSALNNTTNPTAYHKGPVTRLALPYTAPHRVLATAYTGTTTYTASARGDLAHLTTTHARHLPTSFNFGAVKAETITELLVRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 216 - DNA, 2925 bp, Artificial Sequence
  consensus VP1-VP4 subtype O and consensus C3 nucleic acid
atgctgaacggcgagtggaaggccaaggtgcagaagcgcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccaccgccgaggacttcgtgagcggccccaacaccagcggcctggagacccgcgtggtgcaggccgagcgcttcttcaagacccacctgttcgactgggtgaccagcgaccccttcggccgctgctacctgctggagctgcccaccgaccacaagggcgtgtacggcagcctgaccgacagctacgcctacatgcgcaacggctgggacgtggaggtgaccgccgtgggcaaccagttcaacggcggctgcctgctggtggccatggtgcccgagctgtgcagcatcgacaagcgcgagctgtaccagctgaccctgttcccccaccagttcatcaacccccgcaccaacatgaccgcccacatcaccgtgcccttcgtgggcgtgaaccgctacgaccagtacaaggtgcacaagccctggaccctggtggtgatggtggtggcccccctgaccgtgaacaccgagggcgccccccagatcaaggtgtacgccaacatcgcccccaccaacgtgcacgtggccggcgagttccccagcaaggagggcatcttccccgtggcctgcagcgacggctacggcggcctggtgaccaccgaccccaagaccgccgaccccgcctacggcaaggtgttcaaccccccccgcaacatgctgcccggccgcttcaccaacttcctggacgtggccgaggcctgccccaccttcctgcacttcgagggcggcgtgccctacgtgaccaccaagaccgacagcgaccgcgtgctggcccagttcgacctgagcctggccgccaagcacatgagcaacaccttcctggccggcctggcccagtactacacccagtacagcggcaccatcaacctgcacttcatgttcaccggccccaccgacgccaaggcccgctacatgatcgcctacgccccccccggcatggagccccccaagacccccgaggccgccgcccactgcatccacgccgagtgggacaccggcctgaacagcaagttcaccttcagcatcccctacctgagcgccgccgactacgcctacaccgccagcgacgccgccgagaccaccaacgtgcagggctgggtgtgcctgttccagatcacccacggcaaggccgacggcgacgccctggtggtgctggccagcgccggcaaggacttcgagctgcgcctgcccgtggacgcccgcacccagaccaccagcgccggcgagagcgccgaccccgtgaccgccaccgtggagaactacggcggcgagacccaggtgcagcgccgccagcacaccgacgtgagcttcatcctggaccgcttcgtgaaggtgacccccaaggaccagatcaacgtgctggacctgatgcagacccccgcccacaccctggtgggcgccctgctgcgcaccgccacctactacttcgccgacctggaggtggccgtgaagcacgagggcaacctgacctgggtgcccaacggcgcccccgagaccgccctggacaacaccaccaaccccaccgcctaccacaaggcccccctgacccgcctggccctgccctacaccgccccccaccgcgtgctggccaccgtgtacaacggcaactgcaagtacggcgagagccccgtgaccaacgtgcgcggcgacctgcaggtgctggcccagaaggccgcccgcaccctgcccaccagcttcaactacggcgccatcaaggccacccgcgtgaccgagctgctgtaccgcatgaagcgcgccgagacctactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 217 - protein, 974 aa, Artificial Sequence
  consensus VP1-VP4 subtype O and consensus C3 amino acid
MLNGEWKAKVQKRLRGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTYGYATAEDFVSGPNTSGLETRVVQAERFFKTHLFDWVTSDPFGRCYLLELPTDHKGVYGSLTDSYAYMRNGWDVEVTAVGNQFNGGCLLVAMVPELCSIDKRELYQLTLFPHQFINPRTNMTAHITVPFVGVNRYDQYKVHKPWTLVVMVVAPLTVNTEGAPQIKVYANIAPTNVHVAGEFPSKEGIFPVACSDGYGGLVTTDPKTADPAYGKVFNPPRNMLPGRFTNFLDVAEACPTFLHFEGGVPYVTTKTDSDRVLAQFDLSLAAKHMSNTFLAGLAQYYTQYSGTINLHFMFTGPTDAKARYMIAYAPPGMEPPKTPEAAAHCIHAEWDTGLNSKFTFSIPYLSAADYAYTASDAAETTNVQGWVCLFQITHGKADGDALVVLASAGKDFELRLPVDARTQTTSAGESADPVTATVENYGGETQVQRRQHTDVSFILDRFVKVTPKDQINVLDLMQTPAHTLVGALLRTATYYFADLEVAVKHEGNLTWVPNGAPETALDNTTNPTAYHKAPLTRLALPYTAPHRVLATVYNGNCKYGESPVTNVRGDLQVLAQKAARTLPTSFNYGAIKATRVTELLYRMKRAETYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 218 - DNA, 2952 bp, Artificial Sequence
  consensus VP1-VP4 subtype SAT1 and consensus C3 nucleis acid
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccttcagcggcctggtgggcgccctgctggccgacaagaagaccgaggagaccaccctgctggaggaccgcatcatgaccaccagccacggcaccaccaccagcaccacccagagcagcgtgggcgtgacctacggctacgccctggccgacaagttcctgcccggccccaacaccaacggcctggagacccgcgtggagcaggccgagcgcttcttcaagcacaagctgttcgactggaccaccgaccagcagttcggcaccacccacgtgctggagctgcccaccgaccacaagggcatctacggccagctggtggacagccacgcctacatccgcaacggctgggacgtgcaggtgagcgccaccgccacccagttcaacggcggctgcctgctggtggccatggtgcccgagctgtgcaagctggacgaccgcgagaagtaccagctgaccctgttcccccaccagttcctgaacccccgcaccaacaccaccgcccacatccaggtgccctacctgggcgtggaccgccacgaccagggcacccgccacaaggcctggaccctggtggtgatggtggtggccccctacaccaacgaccagaccatcggcagcaccaaggccgaggtgtacgtgaacatcgcccccaccaacgtgtacgtggccggcgagaagcccgccaagcagggcatcctgcccgtggccgtgagcgacggctacggcggcttccagaacaccgaccccaagaccagcgaccccatctacggccacgtgtacaaccccgcccgcaccctgtaccccggccgcttcaccaacctgctggacgtggccgaggcctgccccaccctgctggacttcaacggcgtgccctacgtgcagacccagaacaacagcggcagcaaggtgctggcccgcttcgacctggccttcggccacaagaacatgaagaacacctacatgagcggcctggcccagtacttcgcccagtacagcggcaccctgaacctgcacttcatgtacaccggccccaccaacaacaaggccaagtacatggtggcctacatcccccccggcacccaccccctgcccgagacccccgagatggccagccactgctaccacgccgagtgggacaccggcctgaacagcaccttcaccttcaccgtgccctacatcagcgccgccgactacgcctacacctacgccgacgagcccgagcaggccagcgtgcagggctgggtgggcgtgtaccagatcaccgacacccacgagaaggacggcgccgtgatcgtgaccgtgagcgccggccccgacttcgagttccgcatgcccatcagccccagccgccagaccaccagcgccggcgagggcgccgaccccgtgaccaccgacgtgagcgagcacggcggcgacagccgcaccgcccgccgcgcccacaccgacgtggccttcctgctggaccgcttcaccctggtgggcaagacccaggacaacaagctggtgctggacctgctgaccaccaaggagaagagcctggtgggcgccctgctgcgcgccgccacctactacttcagcgacctggaggtggcctgcgtgggcaccaacaagtgggtgggctggacccccaacggcagccccgtgaagctgagcgaggtgggcgacaaccccgtggtgttcagccacaacggcaccacccgcttcgccctgccctacaccgccccccaccgcgtgctggccaccgtgtacaacggcgactgcaagtacaagcccaccggcacccccccccgcgagaacatccgcggcgacctggccaccctggccgcccgcatcgccagcgagacccacatccccaccaccttcaactacggcatgatctacaccgaggccgaggtggacgtgtacctgcgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 219 - protein, 983 aa, Artificial Sequence
  consensus VP1-VP4 subtype SAT1 and consensus C3 amino acid
MLDVDWQDRAGLFLRGAGQSSPATGSQNQSGNTGSIINNYYMQQYQNSMDTQLGDNAISGGSNEGSTDTTSTHTNNTQNNDWFSKLAQSAFSGLVGALLADKKTEETTLLEDRIMTTSHGTTTSTTQSSVGVTYGYALADKFLPGPNTNGLETRVEQAERFFKHKLFDWTTDQQFGTTHVLELPTDHKGIYGQLVDSHAYIRNGWDVQVSATATQFNGGCLLVAMVPELCKLDDREKYQLTLFPHQFLNPRTNTTAHIQVPYLGVDRHDQGTRHKAWTLVVMVVAPYTNDQTIGSTKAEVYVNIAPTNVYVAGEKPAKQGILPVAVSDGYGGFQNTDPKTSDPIYGHVYNPARTLYPGRFTNLLDVAEACPTLLDFNGVPYVQTQNNSGSKVLARFDLAFGHKNMKNTYMSGLAQYFAQYSGTLNLHFMYTGPTNNKAKYMVAYIPPGTHPLPETPEMASHCYHAEWDTGLNSTFTFTVPYISAADYAYTYADEPEQASVQGWVGVYQITDTHEKDGAVIVTVSAGPDFEFRMPISPSRQTTSAGEGADPVTTDVSEHGGDSRTARRAHTDVAFLLDRFTLVGKTQDNKLVLDLLTTKEKSLVGALLRAATYYFSDLEVACVGTNKWVGWTPNGSPVKLSEVGDNPVVFSHNGTTRFALPYTAPHRVLATVYNGDCKYKPTGTPPRENIRGDLATLAARIASETHIPTTFNYGMIYTEAEVDVYLRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 220 - DNA, 2940 bp, Artificial Sequence
  consensus VP1-VP4 subtype SAT2 and consensus C3 nucleic acid
atgctggacgtggactggcaggacaaggccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccaccctgctggaggaccgcatcctgaccacccgccacggcaccaccaccagcaccacccagagcagcgtgggcatcacctacggctacgccgacgccgacagcttccgccccggccccaacaccagcggcctggagacccgcgtggagcaggccgagcgcttcttcaaggagaagctgttcgactggaccagcgacaagcccttcggcaccctgtacgtgctggagctgcccaaggaccacaagggcatctacggcagcctgaccgacgcctacacctacatgcgcaacggctgggacgtgcaggtgagcgccaccagcacccagttcaacggcggcagcctgctggtggccatggtgcccgagctgtgcagcctgaaggaccgcgaggagttccagctgaccctgtacccccaccagttcatcaacccccgcaccaacaccaccgcccacatccaggtgccctacctgggcgtgaaccgccacgaccagggcaagcgccaccaggcctggagcctggtggtgatggtgctgacccccctgaccaccgaggcccagatgaacagcggcaccgtggaggtgtacgccaacatcgcccccaccaacgtgttcgtggccggcgagaagcccgccaagcagggcatcatccccgtggcctgcgccgacggctacggcggcttccagaacaccgaccccaagaccgccgaccccatctacggctacgtgtacaaccccagccgcaacgactgccacggccgctacagcaacctgctggacgtggccgaggcctgccccaccctgctgaacttcgacggcaagccctacgtggtgaccaagaacaacggcgacaaggtgatggccgccttcgacgtggccttcacccacaaggtgcacaagaacaccttcctggccggcctggccgactactacacccagtaccagggcagcctgaactaccacttcatgtacaccggccccacccaccacaaggccaagttcatggtggcctacatcccccccggcatcgagaccgacaagctgcccaagacccccgaggacgccgcccactgctaccacagcgagtgggacaccggcctgaacagccagttcaccttcgccgtgccctacgtgagcgccagcgacttcagctacacccacaccgacacccccgccatggccaccaccaacggctgggtggccgtgttccaggtgaccgacacccacagcgccgaggccgccgtggtggtgagcgtgagcgccggccccgacctggagttccgcttccccatcgaccccgtgcgccagaccaccagcgccggcgagggcgccgaggtggtgaccaccgaccccagcacccacggcggcaaggtgaccgagaagcgccgcgtgcacaccgacgtggccttcgtgctggaccgcttcacccacgtgcacaccaacaagaccaccttcgccgtggacctgatggacaccaaggagaagaccctggtgggcgccctgctgcgcgccgccacctactacttctgcgacctggagatcgcctgcgtgggcgagcacaagcgcgtgttctggcagcccaacggcgccccccgcaccacccagctgggcgacaaccccatggtgttcagccacaacaaggtgacccgcttcgccatcccctacaccgccccccaccgcctgctgagcaccgtgtacaacggcgagtgcgagtacaccaagaccgtgaccgccatccgcggcgaccgcgaggtgctggccgccaagtacagcagcgccaagcacaccctgcccagcaccttcaacttcggcttcgtgaccgccgacgagcccgtggacgtgtactaccgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 221 - protein, 979 aa, Artificial Sequence
  consensus VP1-VP4 subtype SAT2 and consensus C3 amino acid
MLDVDWQDKAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTLLEDRILTTRHGTTTSTTQSSVGITYGYADADSFRPGPNTSGLETRVEQAERFFKEKLFDWTSDKPFGTLYVLELPKDHKGIYGSLTDAYTYMRNGWDVQVSATSTQFNGGSLLVAMVPELCSLKDREEFQLTLYPHQFINPRTNTTAHIQVPYLGVNRHDQGKRHQAWSLVVMVLTPLTTEAQMNSGTVEVYANIAPTNVFVAGEKPAKQGIIPVACADGYGGFQNTDPKTADPIYGYVYNPSRNDCHGRYSNLLDVAEACPTLLNFDGKPYVVTKNNGDKVMAAFDVAFTHKVHKNTFLAGLADYYTQYQGSLNYHFMYTGPTHHKAKFMVAYIPPGIETDKLPKTPEDAAHCYHSEWDTGLNSQFTFAVPYVSASDFSYTHTDTPAMATTNGWVAVFQVTDTHSAEAAVVVSVSAGPDLEFRFPIDPVRQTTSAGEGAEVVTTDPSTHGGKVTEKRRVHTDVAFVLDRFTHVHTNKTTFAVDLMDTKEKTLVGALLRAATYYFCDLEIACVGEHKRVFWQPNGAPRTTQLGDNPMVFSHNKVTRFAIPYTAPHRLLSTVYNGECEYTKTVTAIRGDREVLAAKYSSAKHTLPSTFNFGFVTADEPVDVYYRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 222 - DNA, 2937 bp, Artificial Sequence
  consensus VP1-VP4 subtype SAT3 and consensus C3 nucleic acid
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccacccacctggaggaccgcatcctgaccacccgccacaacaccaccaccagcaccacccagagcagcgtgggcgtgacctacggctacgtgagcgccgaccgcttcctgcccggccccaacaccagcggcctggagagccgcgtggagcaggccgagcgcttcttcaaggagaagctgttcacctggaccgccagccaggagtacgcccacgtgcacctgctggagctgcccaccgaccacaagggcatctacggcgccatggtggacagccacgcctacgtgcgcaacggctgggacgtgcaggtgaccgccaccagcacccagttcaacggcggcaccctgctggtggccatggtgcccgagctgcacagcctggacacccgcgacgtgagccagctgaccctgttcccccaccagttcatcaacccccgcaccaacaccaccgcccacatcgtggtgccctacgtgggcgtgaaccgccacgaccaggtgcagatgcacaaggcctggaccctggtggtggccgtgatggcccccctgaccaccagcagcatgggccaggacaacgtggaggtgtacgccaacatcgcccccaccaacgtgtacgtggccggcgagcgccccagcaagcagggcatcatccccgtggcctgcaacgacggctacggcggcttccagaacaccgaccccaagaccgccgaccccatctacggcctggtgagcaaccccccccgcaccgccttccccggccgcttcaccaacctgctggacgtggccgaggcctgccccaccttcctggacttcgacggcgtgccctacgtgaagaccacccacaacagcggcagcaagatcctgacccacatcgacctggccttcggccacaagagcttcaagaacacctacctggccggcctggcccagtactacgcccagtacagcggcagcatcaacctgcacttcatgtacaccggccccacccagagcaaggcccgcttcatggtggcctacatcccccccggcaccaccgtgcccaacacccccgagcaggccgcccactgctaccacagcgagtgggacaccggcctgaacagcaagttcaccttcaccgtgccctacatgagcgccgccgacttcgcctacacctactgcgacgagcccgagcaggccagcgcccagggctgggtgaccctgtaccagatcaccgacacccacgaccccgacagcgccgtgctggtgagcgtgagcgccggcgccgacttcgagctgcgcctgcccatcaaccccgccgcccagaccaccagcgccggcgagggcgccgacgtggtgaccaccgacgtgaccacccacggcggcgaggtgagcgtgccccgccgccagcacaccaacgtggagttcctgctggaccgcttcacccacatcggcaccatcaacggccaccgcaccatctgcctgatggacaccaaggagcacaccctggtgggcgccatcctgcgcagcgccacctactacttctgcgacctggaggtggccgtgctgggcaacgccaagtacgccgcctgggtgcccaacggctgcccccacaccgaccgcgtggaggacaaccccgtggtgcacagcaagggcagcgtggtgcgcttcgccctgccctacaccgccccccacggcgtgctggccaccgtgtacaacggcaactgcaagtacagcaccacccagcgcgtggccccccgccgcggcgacctgggcgtgctgagccagcgcgtggagaacgagaccacccgctgcatccccaccaccttcaacttcggccgcctgctgtgcgagagcggcgacgtgtactaccgcatgaagcgcaccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 223 - protein, 978 aa, Artificial Sequence
  consensus VP1-VP4 subtype SAT3 and consensus C3 amino acid
MLDVDWQDRAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTHLEDRILTTRHNTTTSTTQSSVGVTYGYVSADRFLPGPNTSGLESRVEQAERFFKEKLFTWTASQEYAHVHLLELPTDHKGIYGAMVDSHAYVRNGWDVQVTATSTQFNGGTLLVAMVPELHSLDTRDVSQLTLFPHQFINPRTNTTAHIVVPYVGVNRHDQVQMHKAWTLVVAVMAPLTTSSMGQDNVEVYANIAPTNVYVAGERPSKQGIIPVACNDGYGGFQNTDPKTADPIYGLVSNPPRTAFPGRFTNLLDVAEACPTFLDFDGVPYVKTTHNSGSKILTHIDLAFGHKSFKNTYLAGLAQYYAQYSGSINLHFMYTGPTQSKARFMVAYIPPGTTVPNTPEQAAHCYHSEWDTGLNSKFTFTVPYMSAADFAYTYCDEPEQASAQGWVTLYQITDTHDPDSAVLVSVSAGADFELRLPINPAAQTTSAGEGADVVTTDVTTHGGEVSVPRRQHTNVEFLLDRFTHIGTINGHRTICLMDTKEHTLVGAILRSATYYFCDLEVAVLGNAKYAAWVPNGCPHTDRVEDNPVVHSKGSVVRFALPYTAPHGVLATVYNGNCKYSTTQRVAPRRGDLGVLSQRVENETTRCIPTTFNFGRLLCESGDVYYRMKRTELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 224 - DNA, 756 bp, Artificial Sequence
  consensus C3 amino acid
tactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 225 - protein, 251 aa, Artificial Sequence
  consensus C3  amino acid
YCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 226 - DNA, 2160 bp, Artificial Sequence
  consensus VP1-VP4 subtype A nucleic acid
atgctgaacggcgactggaaggccaaggtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcaccggcctgttcggcgccctgctggccgacaagaagaccgaggagaccaccctgctggaggaccgcatcctgaccacccgcaacggccacaccaccagcaccacccagagcagcgtgggcgtgacctacggctacagcaccaccgaggaccacgtggccggccccaacaccagcggcctggagacccgcgtggtgcaggccgagcgcttcttcaagaagttcctgttcgactggaccaccgacaagcccttcggccacctgcacaagctggagctgcccaccgaccaccacggcgtgttcggccacctggtggacagctacgcctacatgcgcaacggctgggacgtggaggtgagcgccgtgggcaaccagttcaacggcggctgcctgctggtggccatggtgcccgagtggaaggagttcgacacccgcgagaagtaccagctgaccctgttcccccaccagttcatcagcccccgcaccaacatgaccgcccacatcaccgtgccctacctgggcgtgaaccgctacgaccagtacaagaagcacaagccctggaccctggtggtgatggtggtgagccccctgaccgtgaacaccgccgcccagatcaaggtgtacgccaacatcgcccccacctacgtgcacgtggccggcgagctgcccagcaaggagggcatcttccccgtggcctgcgccgacggctacggcggcctggtgaccaccgaccccaagaccgccgaccccgcctacggcaaggtgtacaaccccccccgcaccaactaccccggccgcttcaccaacctgctggacgtggccgaggcctgccccaccttcctgtgcttcgacgacggcaagccctacgtgaccacccgcaccgacgagacccgcctgctggccaagttcgacgtgagcctggccgccaagcacatgagcaacacctacctgagcggcatcgcccagtactacacccagtacagcggcaccatcaacctgcacttcatgttcaccggcagcaccgacagcaaggcccgctacatggtggcctacatcccccccggcgtggagaccccccccgacacccccgagcgcgccgcccactgcatccacgccgagtgggacaccggcctgaacagcaagttcaccttcagcatcccctacgtgagcgccgccgactacgcctacaccgccagcgacaccgccgagaccaccaacgtgcagggctgggtgtgcgtgtaccagatcacccacggcaaggccgagaacgacaccctggtggtgagcgtgagcgccggcaaggacttcgagctgcgcctgcccatcgacccccgccagcagaccaccgccaccggcgagagcgccgaccccgtgaccaccaccgtggagaactacggcggcgagacccaggtgcagcgccgccaccacaccgacgtgggcttcatcatggaccgcttcgtgaagatcaacagccccaagcccacccacgtgatcgacctgatgcagacccaccagcacggcctggtgggcgccctgctgcgcgccgccacctactacttcagcgacctggagatcgtggtgcgccacgacggcctgacctgggtgcccaacggcgcccccgagagcgccctgagcaacaccagcaaccccaccgcctacaacaaggcccccttcacccgcctggccctgccctacaccgccccccaccgcgtgctggccaccgtgtacaacggcaccagcaagtacaccgtgagcggcagcagccgccgcggcgacctgggcagcctggccgcccgcgtggccaagcagctgcccgccagcttcaactacggcgccatcaaggccgacaccatccacgagctgctggtgcgcatgaagcgcgccgagctg

SEQ ID NO: 227 - protein, 720 aa, Artificial Sequence
  consensus VP1-VP4 subtype A amino acid
MLNGDWKAKVQRKLKGAGQSSPATGSQNQSGNTGSIINNYYMQQYQNSMDTQLGDNAISGGSNEGSTDTTSTHTTNTQNNDWFSKLASSAFTGLFGALLADKKTEETTLLEDRILTTRNGHTTSTTQSSVGVTYGYSTTEDHVAGPNTSGLETRVVQAERFFKKFLFDWTTDKPFGHLHKLELPTDHHGVFGHLVDSYAYMRNGWDVEVSAVGNQFNGGCLLVAMVPEWKEFDTREKYQLTLFPHQFISPRTNMTAHITVPYLGVNRYDQYKKHKPWTLVVMVVSPLTVNTAAQIKVYANIAPTYVHVAGELPSKEGIFPVACADGYGGLVTTDPKTADPAYGKVYNPPRTNYPGRFTNLLDVAEACPTFLCFDDGKPYVTTRTDETRLLAKFDVSLAAKHMSNTYLSGIAQYYTQYSGTINLHFMFTGSTDSKARYMVAYIPPGVETPPDTPERAAHCIHAEWDTGLNSKFTFSIPYVSAADYAYTASDTAETTNVQGWVCVYQITHGKAENDTLVVSVSAGKDFELRLPIDPRQQTTATGESADPVTTTVENYGGETQVQRRHHTDVGFIMDRFVKINSPKPTHVIDLMQTHQHGLVGALLRAATYYFSDLEIVVRHDGLTWVPNGAPESALSNTSNPTAYNKAPFTRLALPYTAPHRVLATVYNGTSKYTVSGSSRRGDLGSLAARVAKQLPASFNYGAIKADTIHELLVRMKRAEL

SEQ ID NO: 228 - DNA, 2160 bp, Artificial Sequence
  consensus VP1-VP4 subtype Asia1 nucleic acid
atgctgaacggcgagtggaaggccaaggtgcagaagcgcctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagccgcctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccgtggccgaggacgccgtgagcggccccaacaccagcggcctggagacccgcgtgcagcaggccgagcgcttcttcaagaagcacctgttcgactggacccccaacctggccttcggccactgccactacctggagctgcccaccgagcacaagggcgtgtacggcagcctgatggacagctacgcctacatgcgcaacggctgggacatcgaggtgaccgccgtgggcaaccagttcaacggcggctgcctgctggtggccctggtgcccgagctgaagagcctggacacccgccagaagtaccagctgaccctgttcccccaccagttcatcaacccccgcaccaacatgaccgcccacatcaacgtgcccttcgtgggcgtgaaccgctacgaccagtacgccctgcacaagccctggaccctggtggtgatggtggtggcccccctgaccgtgaagaccggcggcagcgagcagatcaaggtgtacatgaacgccgcccccacctacgtgcacgtggccggcgagctgcccagcaaggagggcatcgtgcccgtggcctgcgccgacggctacggcaacatggtgaccaccgaccccaagaccgccgaccccgtgtacggcaaggtgttcaaccccccccgcaccaacctgcccggccgcttcaccaacttcctggacgtggccgaggcctgccccaccttcctgcgcttcggcgaggtgcccttcgtgaagaccgtgaacagcggcgaccgcctgctggccaagttcgacgtgagcctggccgccggccacatgagcaacacctacctggccggcctggcccagtactacacccagtacagcggcaccatgaacgtgcacttcatgttcaccggccccaccgacgccaaggcccgctacatggtggcctacatcccccccggcatgaccccccccaccgaccccgagcgcgccgcccactgcatccacagcgagtgggacaccggcctgaacagcaagttcaccttcagcatcccctacctgagcgccgccgactacgcctacaccgccagcgacaccgccgagaccaccagcgtgcagggctgggtgtgcatctaccagatcacccacggcaaggccgagggcgacgccctggtggtgagcgtgagcgccggcaaggacttcgagttccgcctgcccgtggacgcccgccgccagaccaccaccaccggcgagagcgccgaccccgtgaccaccaccgtggagaactacggcggcgagacccagaccgcccgccgcctgcacaccgacgtggccttcgtgctggaccgcttcgtgaagctgaccgcccccaagaacacccagaccctggacctgatgcagatccccagccacaccctggtgggcgccctgctgcgcagcgccacctactacttcagcgacctggaggtggccctggtgcacaccggccccgtgacctgggtgcccaacggcagccccaaggacgccctggacaaccagaccaaccccaccgcctaccagaagcagcccatcacccgcctggccctgccctacaccgccccccaccgcgtgctggccaccgtgtacaacggcaagaccacctacggcgagacccccagccgccgcggcgacatggccgccctggcccagcgcctgagcgagcgcctgcccaccagcttcaactacggcgccgtgaaggccgagaccatcaccgagctgctgatccgcatgaagcgcgccgagacc

SEQ ID NO: 229 - protein, 720 aa, Artificial Sequence
  consensus VP1-VP4 subtype Asia 1 amino acid
MLNGEWKAKVQKRLKGAGQSSPATGSQNQSGNTGSIINNYYMQQYQNSMDTQLGDNAISGGSNEGSTDTTSTHTNNTQNNDWFSRLASSAFSGLFGALLADKKTEETTLLEDRILTTRNGHTTSTTQSSVGVTYGYAVAEDAVSGPNTSGLETRVQQAERFFKKHLFDWTPNLAFGHCHYLELPTEHKGVYGSLMDSYAYMRNGWDIEVTAVGNQFNGGCLLVALVPELKSLDTRQKYQLTLFPHQFINPRTNMTAHINVPFVGVNRYDQYALHKPWTLVVMVVAPLTVKTGGSEQIKVYMNAAPTYVHVAGELPSKEGIVPVACADGYGNMVTTDPKTADPVYGKVFNPPRTNLPGRFTNFLDVAEACPTFLRFGEVPFVKTVNSGDRLLAKFDVSLAAGHMSNTYLAGLAQYYTQYSGTMNVHFMFTGPTDAKARYMVAYIPPGMTPPTDPERAAHCIHSEWDTGLNSKFTFSIPYLSAADYAYTASDTAETTSVQGWVCIYQITHGKAEGDALVVSVSAGKDFEFRLPVDARRQTTTTGESADPVTTTVENYGGETQTARRLHTDVAFVLDRFVKLTAPKNTQTLDLMQIPSHTLVGALLRSATYYFSDLEVALVHTGPVTWVPNGSPKDALDNQTNPTAYQKQPITRLALPYTAPHRVLATVYNGKTTYGETPSRRGDMAALAQRLSERLPTSFNYGAVKAETITELLIRMKRAET

SEQ ID NO: 230 - DNA, 2157 bp, Artificial Sequence
  consensus VP1-VP4 subtype C nucleic acid
atgctgaacgagggctggaaggccagcgtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgaccttcggctacgccaccgccgaggacagcaccagcggccccaacaccagcggcctggagacccgcgtgcaccaggccgagcgcttcttcaagatggccctgttcgactgggtgcccagccagaacttcggccacatgcacaaggtggtgctgccccacgagcccaagggcgtgtacggcggcctggtgaagagctacgcctacatgcgcaacggctgggacgtggaggtgaccgccgtgggcaaccagttcaacggcggctgcctgctggtggccctggtgcccgagatgggcgacatcagcgaccgcgagaagtaccagctgaccctgtacccccaccagttcatcaacccccgcaccaacatgaccgcccacatcaccgtgccctacgtgggcgtgaaccgctacgaccagtacaagcagcaccgcccctggaccctggtggtgatggtggtggcccccctgaccaccaacaccgccggcgcccagcagatcaaggtgtacgccaacatcgcccccaccaacgtgcacgtggccggcgagctgcccagcaaggagggcatcttccccgtggcctgcagcgacggctacggcaacatggtgaccaccgaccccaagaccgccgaccccgtgtacggcaaggtgtacaaccccccccgcaccgccctgcccggccgcttcaccaactacctggacgtggccgaggcctgccccaccttcctgatgttcgagaacgtgccctacgtgagcacccgcaccgacggccagcgcctgctggccaagttcgacgtgagcctggccgccaagcacatgagcaacacctacctggccggcctggcccagtactacacccagtacaccggcaccatcaacctgcacttcatgttcaccggccccaccgacgccaaggcccgctacatggtggcctacgtgccccccggcatggacgcccccgacaaccccgaggaggccgcccactgcatccacgccgagtgggacaccggcctgaacagcaagttcaccttcagcatcccctacatcagcgccgccgactacgcctacaccgccagccacaaggccgagaccacctgcgtgcagggctgggtgtgcgtgtaccagatcacccacggcaaggccgacgccgacgccctggtggtgagcgccagcgccggcaaggacttcgagctgcgcctgcccgtggacgcccgcaagcagaccaccaccaccggcgagagcgccgaccccgtgaccaccaccgtggagaactacggcggcgagacccaggtgcagcgccgccaccacaccgacgtggccttcgtgctggaccgcttcgtggaggtgaccgtgagcggccgcaaccagcacaccctggacgtgatgcaggcccacaaggacaacatcgtgggcgccctgctgcgcgccgccacctactacttcagcgacctggagatcgccgtgacccacaccggcaagctgacctgggtgcccaacggcgcccccgtgagcgccctgaacaacaccaccaaccccaccgcctaccacaagggccccgtgacccgcctggccctgccctacaccgccccccaccgcgtgctggccaccgcctacaccggcaccaccacctacaccgccagcgcccgcggcgacctggcccacctgaccaccacccacgcccgccacctgcccaccagcttcaacttcggcgccgtgaaggccgagaccatcaccgagctgctggtgcgcatgaagcgcgccgagctg

SEQ ID NO: 231 - protein, 719 aa, Artificial Sequence
  consensus VP1-VP4 subtype C amino acid
MLNEGWKASVQRKLKGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTFGYATAEDSTSGPNTSGLETRVHQAERFFKMALFDWVPSQNFGHMHKVVLPHEPKGVYGGLVKSYAYMRNGWDVEVTAVGNQFNGGCLLVALVPEMGDISDREKYQLTLYPHQFINPRTNMTAHITVPYVGVNRYDQYKQHRPWTLVVMVVAPLTTNTAGAQQIKVYANIAPTNVHVAGELPSKEGIFPVACSDGYGNMVTTDPKTADPVYGKVYNPPRTALPGRFTNYLDVAEACPTFLMFENVPYVSTRTDGQRLLAKFDVSLAAKHMSNTYLAGLAQYYTQYTGTINLHFMFTGPTDAKARYMVAYVPPGMDAPDNPEEAAHCIHAEWDTGLNSKFTFSIPYISAADYAYTASHKAETTCVQGWVCVYQITHGKADADALVVSASAGKDFELRLPVDARKQTTTTGESADPVTTTVENYGGETQVQRRHHTDVAFVLDRFVEVTVSGRNQHTLDVMQAHKDNIVGALLRAATYYFSDLEIAVTHTGKLTWVPNGAPVSALNNTTNPTAYHKGPVTRLALPYTAPHRVLATAYTGTTTYTASARGDLAHLTTTHARHLPTSFNFGAVKAETITELLVRMKRAEL

SEQ ID NO: 232 - DNA, 2169 bp, Artificial Sequence
  consensus VP1-VP4 subtype O nucleic acid
atgctgaacggcgagtggaaggccaaggtgcagaagcgcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccaccgccgaggacttcgtgagcggccccaacaccagcggcctggagacccgcgtggtgcaggccgagcgcttcttcaagacccacctgttcgactgggtgaccagcgaccccttcggccgctgctacctgctggagctgcccaccgaccacaagggcgtgtacggcagcctgaccgacagctacgcctacatgcgcaacggctgggacgtggaggtgaccgccgtgggcaaccagttcaacggcggctgcctgctggtggccatggtgcccgagctgtgcagcatcgacaagcgcgagctgtaccagctgaccctgttcccccaccagttcatcaacccccgcaccaacatgaccgcccacatcaccgtgcccttcgtgggcgtgaaccgctacgaccagtacaaggtgcacaagccctggaccctggtggtgatggtggtggcccccctgaccgtgaacaccgagggcgccccccagatcaaggtgtacgccaacatcgcccccaccaacgtgcacgtggccggcgagttccccagcaaggagggcatcttccccgtggcctgcagcgacggctacggcggcctggtgaccaccgaccccaagaccgccgaccccgcctacggcaaggtgttcaaccccccccgcaacatgctgcccggccgcttcaccaacttcctggacgtggccgaggcctgccccaccttcctgcacttcgagggcggcgtgccctacgtgaccaccaagaccgacagcgaccgcgtgctggcccagttcgacctgagcctggccgccaagcacatgagcaacaccttcctggccggcctggcccagtactacacccagtacagcggcaccatcaacctgcacttcatgttcaccggccccaccgacgccaaggcccgctacatgatcgcctacgccccccccggcatggagccccccaagacccccgaggccgccgcccactgcatccacgccgagtgggacaccggcctgaacagcaagttcaccttcagcatcccctacctgagcgccgccgactacgcctacaccgccagcgacgccgccgagaccaccaacgtgcagggctgggtgtgcctgttccagatcacccacggcaaggccgacggcgacgccctggtggtgctggccagcgccggcaaggacttcgagctgcgcctgcccgtggacgcccgcacccagaccaccagcgccggcgagagcgccgaccccgtgaccgccaccgtggagaactacggcggcgagacccaggtgcagcgccgccagcacaccgacgtgagcttcatcctggaccgcttcgtgaaggtgacccccaaggaccagatcaacgtgctggacctgatgcagacccccgcccacaccctggtgggcgccctgctgcgcaccgccacctactacttcgccgacctggaggtggccgtgaagcacgagggcaacctgacctgggtgcccaacggcgcccccgagaccgccctggacaacaccaccaaccccaccgcctaccacaaggcccccctgacccgcctggccctgccctacaccgccccccaccgcgtgctggccaccgtgtacaacggcaactgcaagtacggcgagagccccgtgaccaacgtgcgcggcgacctgcaggtgctggcccagaaggccgcccgcaccctgcccaccagcttcaactacggcgccatcaaggccacccgcgtgaccgagctgctgtaccgcatgaagcgcgccgagacc

SEQ ID NO: 233 - protein, 723 aa, Artificial Sequence
  Consensus VPI-VP4 subtype 0 nucleic acid
MLNGEWKAKVQKRLRGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTYGYATAEDFVSGPNTSGLETRVVQAERFFKTHLFDWVTSDPFGRCYLLELPTDHKGVYGSLTDSYAYMRNGWDVEVTAVGNQFNGGCLLVAMVPELCSIDKRELYQLTLFPHQFINPRTNMTAHITVPFVGVNRYDQYKVHKPWTLVVMVVAPLTVNTEGAPQIKVYANIAPTNVHVAGEFPSKEGIFPVACSDGYGGLVTTDPKTADPAYGKVFNPPRNMLPGRFTNFLDVAEACPTFLHFEGGVPYVTTKTDSDRVLAQFDLSLAAKHMSNTFLAGLAQYYTQYSGTINLHFMFTGPTDAKARYMIAYAPPGMEPPKTPEAAAHCIHAEWDTGLNSKFTFSIPYLSAADYAYTASDAAETTNVQGWVCLFQITHGKADGDALVVLASAGKDFELRLPVDARTQTTSAGESADPVTATVENYGGETQVQRRQHTDVSFILDRFVKVTPKDQINVLDLMQTPAHTLVGALLRTATYYFADLEVAVKHEGNLTWVPNGAPETALDNTTNPTAYHKAPLTRLALPYTAPHRVLATVYNGNCKYGESPVTNVRGDLQVLAQKAARTLPTSFNYGAIKATRVTELLYRMKRAET

SEQ ID NO: 234 - DNA, 2196 bp, Artificial Sequence
  consensus VP1-VP4 subtype SAT1 nucleic acid
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccttcagcggcctggtgggcgccctgctggccgacaagaagaccgaggagaccaccctgctggaggaccgcatcatgaccaccagccacggcaccaccaccagcaccacccagagcagcgtgggcgtgacctacggctacgccctggccgacaagttcctgcccggccccaacaccaacggcctggagacccgcgtggagcaggccgagcgcttcttcaagcacaagctgttcgactggaccaccgaccagcagttcggcaccacccacgtgctggagctgcccaccgaccacaagggcatctacggccagctggtggacagccacgcctacatccgcaacggctgggacgtgcaggtgagcgccaccgccacccagttcaacggcggctgcctgctggtggccatggtgcccgagctgtgcaagctggacgaccgcgagaagtaccagctgaccctgttcccccaccagttcctgaacccccgcaccaacaccaccgcccacatccaggtgccctacctgggcgtggaccgccacgaccagggcacccgccacaaggcctggaccctggtggtgatggtggtggccccctacaccaacgaccagaccatcggcagcaccaaggccgaggtgtacgtgaacatcgcccccaccaacgtgtacgtggccggcgagaagcccgccaagcagggcatcctgcccgtggccgtgagcgacggctacggcggcttccagaacaccgaccccaagaccagcgaccccatctacggccacgtgtacaaccccgcccgcaccctgtaccccggccgcttcaccaacctgctggacgtggccgaggcctgccccaccctgctggacttcaacggcgtgccctacgtgcagacccagaacaacagcggcagcaaggtgctggcccgcttcgacctggccttcggccacaagaacatgaagaacacctacatgagcggcctggcccagtacttcgcccagtacagcggcaccctgaacctgcacttcatgtacaccggccccaccaacaacaaggccaagtacatggtggcctacatcccccccggcacccaccccctgcccgagacccccgagatggccagccactgctaccacgccgagtgggacaccggcctgaacagcaccttcaccttcaccgtgccctacatcagcgccgccgactacgcctacacctacgccgacgagcccgagcaggccagcgtgcagggctgggtgggcgtgtaccagatcaccgacacccacgagaaggacggcgccgtgatcgtgaccgtgagcgccggccccgacttcgagttccgcatgcccatcagccccagccgccagaccaccagcgccggcgagggcgccgaccccgtgaccaccgacgtgagcgagcacggcggcgacagccgcaccgcccgccgcgcccacaccgacgtggccttcctgctggaccgcttcaccctggtgggcaagacccaggacaacaagctggtgctggacctgctgaccaccaaggagaagagcctggtgggcgccctgctgcgcgccgccacctactacttcagcgacctggaggtggcctgcgtgggcaccaacaagtgggtgggctggacccccaacggcagccccgtgaagctgagcgaggtgggcgacaaccccgtggtgttcagccacaacggcaccacccgcttcgccctgccctacaccgccccccaccgcgtgctggccaccgtgtacaacggcgactgcaagtacaagcccaccggcacccccccccgcgagaacatccgcggcgacctggccaccctggccgcccgcatcgccagcgagacccacatccccaccaccttcaactacggcatgatctacaccgaggccgaggtggacgtgtacctgcgcatgaagcgcgccgagctg

SEQ ID NO: 235 - protein, 732 aa, Artificial Sequence
  consensus VP1-VP4 subtype SAT1 amino acid
MLDVDWQDRAGLFLRGAGQSSPATGSQNQSGNTGSIINNYYMQQYQNSMDTQLGDNAISGGSNEGSTDTTSTHTNNTQNNDWFSKLAQSAFSGLVGALLADKKTEETTLLEDRIMTTSHGTTTSTTQSSVGVTYGYALADKFLPGPNTNGLETRVEQAERFFKHKLFDWTTDQQFGTTHVLELPTDHKGIYGQLVDSHAYIRNGWDVQVSATATQFNGGCLLVAMVPELCKLDDREKYQLTLFPHQFLNPRTNTTAHIQVPYLGVDRHDQGTRHKAWTLVVMVVAPYTNDQTIGSTKAEVYVNIAPTNVYVAGEKPAKQGILPVAVSDGYGGFQNTDPKTSDPIYGHVYNPARTLYPGRFTNLLDVAEACPTLLDFNGVPYVQTQNNSGSKVLARFDLAFGHKNMKNTYMSGLAQYFAQYSGTLNLHFMYTGPTNNKAKYMVAYIPPGTHPLPETPEMASHCYHAEWDTGLNSTFTFTVPYISAADYAYTYADEPEQASVQGWVGVYQITDTHEKDGAVIVTVSAGPDFEFRMPISPSRQTTSAGEGADPVTTDVSEHGGDSRTARRAHTDVAFLLDRFTLVGKTQDNKLVLDLLTTKEKSLVGALLRAATYYFSDLEVACVGTNKWVGWTPNGSPVKLSEVGDNPVVFSHNGTTRFALPYTAPHRVLATVYNGDCKYKPTGTPPRENIRGDLATLAARIASETHIPTTFNYGMIYTEAEVDVYLRMKRAEL

SEQ ID NO: 236 - DNA, 2184 bp, Artificial Sequence
  consensus VP1-VP4 subtype SAT2 nucleic acid
atgctggacgtggactggcaggacaaggccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccaccctgctggaggaccgcatcctgaccacccgccacggcaccaccaccagcaccacccagagcagcgtgggcatcacctacggctacgccgacgccgacagcttccgccccggccccaacaccagcggcctggagacccgcgtggagcaggccgagcgcttcttcaaggagaagctgttcgactggaccagcgacaagcccttcggcaccctgtacgtgctggagctgcccaaggaccacaagggcatctacggcagcctgaccgacgcctacacctacatgcgcaacggctgggacgtgcaggtgagcgccaccagcacccagttcaacggcggcagcctgctggtggccatggtgcccgagctgtgcagcctgaaggaccgcgaggagttccagctgaccctgtacccccaccagttcatcaacccccgcaccaacaccaccgcccacatccaggtgccctacctgggcgtgaaccgccacgaccagggcaagcgccaccaggcctggagcctggtggtgatggtgctgacccccctgaccaccgaggcccagatgaacagcggcaccgtggaggtgtacgccaacatcgcccccaccaacgtgttcgtggccggcgagaagcccgccaagcagggcatcatccccgtggcctgcgccgacggctacggcggcttccagaacaccgaccccaagaccgccgaccccatctacggctacgtgtacaaccccagccgcaacgactgccacggccgctacagcaacctgctggacgtggccgaggcctgccccaccctgctgaacttcgacggcaagccctacgtggtgaccaagaacaacggcgacaaggtgatggccgccttcgacgtggccttcacccacaaggtgcacaagaacaccttcctggccggcctggccgactactacacccagtaccagggcagcctgaactaccacttcatgtacaccggccccacccaccacaaggccaagttcatggtggcctacatcccccccggcatcgagaccgacaagctgcccaagacccccgaggacgccgcccactgctaccacagcgagtgggacaccggcctgaacagccagttcaccttcgccgtgccctacgtgagcgccagcgacttcagctacacccacaccgacacccccgccatggccaccaccaacggctgggtggccgtgttccaggtgaccgacacccacagcgccgaggccgccgtggtggtgagcgtgagcgccggccccgacctggagttccgcttccccatcgaccccgtgcgccagaccaccagcgccggcgagggcgccgaggtggtgaccaccgaccccagcacccacggcggcaaggtgaccgagaagcgccgcgtgcacaccgacgtggccttcgtgctggaccgcttcacccacgtgcacaccaacaagaccaccttcgccgtggacctgatggacaccaaggagaagaccctggtgggcgccctgctgcgcgccgccacctactacttctgcgacctggagatcgcctgcgtgggcgagcacaagcgcgtgttctggcagcccaacggcgccccccgcaccacccagctgggcgacaaccccatggtgttcagccacaacaaggtgacccgcttcgccatcccctacaccgccccccaccgcctgctgagcaccgtgtacaacggcgagtgcgagtacaccaagaccgtgaccgccatccgcggcgaccgcgaggtgctggccgccaagtacagcagcgccaagcacaccctgcccagcaccttcaacttcggcttcgtgaccgccgacgagcccgtggacgtgtactaccgcatgaagcgcgccgagctg

SEQ ID NO: 237 - protein, 728 aa, Artificial Sequence
  consensus VP1-VP4 subtype SAT2 amino acid
MLDVDWQDKAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTLLEDRILTTRHGTTTSTTQSSVGITYGYADADSFRPGPNTSGLETRVEQAERFFKEKLFDWTSDKPFGTLYVLELPKDHKGIYGSLTDAYTYMRNGWDVQVSATSTQFNGGSLLVAMVPELCSLKDREEFQLTLYPHQFINPRTNTTAHIQVPYLGVNRHDQGKRHQAWSLVVMVLTPLTTEAQMNSGTVEVYANIAPTNVFVAGEKPAKQGIIPVACADGYGGFQNTDPKTADPIYGYVYNPSRNDCHGRYSNLLDVAEACPTLLNFDGKPYVVTKNNGDKVMAAFDVAFTHKVHKNTFLAGLADYYTQYQGSLNYHFMYTGPTHHKAKFMVAYIPPGIETDKLPKTPEDAAHCYHSEWDTGLNSQFTFAVPYVSASDFSYTHTDTPAMATTNGWVAVFQVTDTHSAEAAVVVSVSAGPDLEFRFPIDPVRQTTSAGEGAEVVTTDPSTHGGKVTEKRRVHTDVAFVLDRFTHVHTNKTTFAVDLMDTKEKTLVGALLRAATYYFCDLEIACVGEHKRVFWQPNGAPRTTQLGDNPMVFSHNKVTRFAIPYTAPHRLLSTVYNGECEYTKTVTAIRGDREVLAAKYSSAKHTLPSTFNFGFVTADEPVDVYYRMKRAEL

SEQ ID NO: 238 - DNA, 2181 bp, Artificial Sequence
  consensus VP1-VP4 subtype SAT3 nucleic acid
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccacccacctggaggaccgcatcctgaccacccgccacaacaccaccaccagcaccacccagagcagcgtgggcgtgacctacggctacgtgagcgccgaccgcttcctgcccggccccaacaccagcggcctggagagccgcgtggagcaggccgagcgcttcttcaaggagaagctgttcacctggaccgccagccaggagtacgcccacgtgcacctgctggagctgcccaccgaccacaagggcatctacggcgccatggtggacagccacgcctacgtgcgcaacggctgggacgtgcaggtgaccgccaccagcacccagttcaacggcggcaccctgctggtggccatggtgcccgagctgcacagcctggacacccgcgacgtgagccagctgaccctgttcccccaccagttcatcaacccccgcaccaacaccaccgcccacatcgtggtgccctacgtgggcgtgaaccgccacgaccaggtgcagatgcacaaggcctggaccctggtggtggccgtgatggcccccctgaccaccagcagcatgggccaggacaacgtggaggtgtacgccaacatcgcccccaccaacgtgtacgtggccggcgagcgccccagcaagcagggcatcatccccgtggcctgcaacgacggctacggcggcttccagaacaccgaccccaagaccgccgaccccatctacggcctggtgagcaaccccccccgcaccgccttccccggccgcttcaccaacctgctggacgtggccgaggcctgccccaccttcctggacttcgacggcgtgccctacgtgaagaccacccacaacagcggcagcaagatcctgacccacatcgacctggccttcggccacaagagcttcaagaacacctacctggccggcctggcccagtactacgcccagtacagcggcagcatcaacctgcacttcatgtacaccggccccacccagagcaaggcccgcttcatggtggcctacatcccccccggcaccaccgtgcccaacacccccgagcaggccgcccactgctaccacagcgagtgggacaccggcctgaacagcaagttcaccttcaccgtgccctacatgagcgccgccgacttcgcctacacctactgcgacgagcccgagcaggccagcgcccagggctgggtgaccctgtaccagatcaccgacacccacgaccccgacagcgccgtgctggtgagcgtgagcgccggcgccgacttcgagctgcgcctgcccatcaaccccgccgcccagaccaccagcgccggcgagggcgccgacgtggtgaccaccgacgtgaccacccacggcggcgaggtgagcgtgccccgccgccagcacaccaacgtggagttcctgctggaccgcttcacccacatcggcaccatcaacggccaccgcaccatctgcctgatggacaccaaggagcacaccctggtgggcgccatcctgcgcagcgccacctactacttctgcgacctggaggtggccgtgctgggcaacgccaagtacgccgcctgggtgcccaacggctgcccccacaccgaccgcgtggaggacaaccccgtggtgcacagcaagggcagcgtggtgcgcttcgccctgccctacaccgccccccacggcgtgctggccaccgtgtacaacggcaactgcaagtacagcaccacccagcgcgtggccccccgccgcggcgacctgggcgtgctgagccagcgcgtggagaacgagaccacccgctgcatccccaccaccttcaacttcggccgcctgctgtgcgagagcggcgacgtgtactaccgcatgaagcgcaccgagctg

SEQ ID NO: 239 - protein, 727 aa, Artificial Sequence
  consensus VP1-VP4 subtype SAT3 amino acid
MLDVDWQDRAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTHLEDRILTTRHNTTTSTTQSSVGVTYGYVSADRFLPGPNTSGLESRVEQAERFFKEKLFTWTASQEYAHVHLLELPTDHKGIYGAMVDSHAYVRNGWDVQVTATSTQFNGGTLLVAMVPELHSLDTRDVSQLTLFPHQFINPRTNTTAHIVVPYVGVNRHDQVQMHKAWTLVVAVMAPLTTSSMGQDNVEVYANIAPTNVYVAGERPSKQGIIPVACNDGYGGFQNTDPKTADPIYGLVSNPPRTAFPGRFTNLLDVAEACPTFLDFDGVPYVKTTHNSGSKILTHIDLAFGHKSFKNTYLAGLAQYYAQYSGSINLHFMYTGPTQSKARFMVAYIPPGTTVPNTPEQAAHCYHSEWDTGLNSKFTFTVPYMSAADFAYTYCDEPEQASAQGWVTLYQITDTHDPDSAVLVSVSAGADFELRLPINPAAQTTSAGEGADVVTTDVTTHGGEVSXPRRQHTNXEFLLDRFTHIGTINGHRTICLMDTKEHTLVGAILRSATYYFCDLEVAVLGNAKYAAWVPNGCLHTDRVEDNPVVHSKGSVVRFALPYTAPHGVLATVYNGNCKYSTTQRVAPRRGDLGVLSQRVENETTRCIPTTFNFGRLLCESGDVYYRMKRTEL

SEQ ID NO: 240 - DNA, 678 bp, Artificial Sequence
  VP1 Asia  subtype nucleic acid
atggactggacctggatcctgttcctggtcgccgctgccactagggtgcacagcaccaccaccaccggcgagagcgccgaccccgtgaccaccaccgtggagaactacggcggcgagacacagaccgccaggcgcctccacaccgacgtggccttcgtgctggacagattcgtgaagctgacccagcccaagagcacccagaccctggacctgatgcagatccccagccacaccctcgtgggcgccctgctgagaagcgccacctactacttcagcgacctggaagtggccctggtgcacaccggccctgtgacctgggtgcccaacggcgctcccaagaccgccctgaacaaccacaccaaccccaccgcctaccagaagcagcccatcaccaggctggccctgccctacaccgcccctcacagggtgctgtccaccgtgtacaacggcaagaccacctacggcgaggaaagcagcagaaggggcgatctggccgctctcgccaggcgcgtgaacaacaggctgcccacctccttcaactatggcgccgtcaaggccgacaccatcaccgagctgctgatcaggatgaagagggccgagacatactgccccaggcccctgctggccctggacaccacccaggacaggcgcaagcaggaaatcattgcccccgagaag

SEQ ID NO: 241 - protein, 229 aa, Artificial Sequence
  VP1 Asia  subtype amino acid
MDWTWILFLVAAATRVHSTTTTGESADPVTTTVENYGGETQTARRLHTDVAFVLDRFVKLTQPKSTQTLDLMQIPSHTLVGALLRSATYYFSDLEVALVHTGPVTWVPNGAPKTALNNHTNPTAYQKQPITRLALPYTAPHRVLSTVYNGKTTYGEESSRRGDLAALARRVNNRLPTSFNYGAVKADTITELLIRMKRAETYCPRPLLALDTTQDRRKQEIIAPEKQTL

SEQ ID NO: 242 - DNA, 742 bp, Artificial Sequence
  VP1 O  subtype nucleic acid
atggactggacctggatcctgttcctggtcgccgctgccactagggtgcacagcaccacctctaccggggagtccgccgatcctgtgacagccacagtggaaaattacggcggggaaacccaggtgcagaggcggcagcacaccgatgtgtctttcatcctggaccgcttcgtgaaagtgacccccaaggccgaccagatcaacgtgctggatctcatgcagattcccgcccatacactcgtcggggctctgctgcgcaccgccacatactatttcgccgatctcgaggtggccgtgaagcacgagggcaacctgacatgggtgccaaatggcgcccctgaggccgctctggacaacaccaccaatcctacagcctaccacaaggcccccctgaccagactggctctgccttatacagccccccaccgcgtgctggccacagtgtataatggcaactgcaagtacggcgaagtggccgtcaccaacgtgcgcggcgacctccaggtgctggcccagaaggccgccaggaccctgcctaccagctttaactacggggccatcaaggccaccagagtgaccgaactgctgtacagaatgaagcgcgccgaaacctactgccctagacctctgctcgccatccaccccagcgaggccaggcacaagcagaaaattgtggcccctgtgaagcagctgctgtgatgactgcagatatccagcacagtggcggccgctcgagtctaga

SEQ ID NO: 243 - protein, 201 aa, Artificial Sequence
  VP1 O  subtype amino acid
MDWTWILFLVAAATRVHSTTSTGESADPVTATVENYGGETQVQRRQHTDVSFILDRFVKVTPKADQINVLDLMQIPAHTLVGALLRTATYYFADLEVAVKHEGNLTWVPNGAPEAALDNTTNPTAYHKAPLTRLALPYTAPHRVLATVYNGNCKYGEVAVTNVRGDLQVLAQKAARTLPTSFNYGAIKATRVTELLYRMKR

SEQ ID NO: 244 - DNA, 771 bp, Artificial Sequence
  VP1 A subtype nucleic acid
atggactggacctggattctgtttctggtggccgctgccacaagagtgcactccaccacctctgccggcgagtccgccgacccagtgaccaccaccgtggagaactacggcggcgagacacaggtgcagcgcaggcaccacaccgacgtgggcttcatcatggaccgcttcgtgaagatcggcaacacctcccccacccacgtgatcgacctgatgcagacccaccagcacggactggtgggagccctgctgagagccgccacctactacttctccgacctggaaatcgtggtgcgccacgacggaaacctgacatgggtgcccaatggcgccccagaggccgccctgtccaacaccggcaaccccaccgcctacaacaaggcccccttcaccagactggccctgccatacaccgcccctcacagggtgctggccaccgtgtacaacggcaccaacaagtactccgccgcctccggaagaacaagaggcgacctgggcaccgtggccgccagaatcgccgcccagctgcccgcctccttcaacttcggcgccatcaaggccgacgccatccacgaactgctggtgcgcatgaagcgcgccgagctgtactgcccaagacccctgctggccgtggaggtgtcctcccaggaccgccacaagcagaagatcatcgccccagccaagcagctgctgtacccctacgacgtgcccgactacgcctccctgggcggaccatgatgactcgagtctagagggcccgtttaaacccgc

SEQ ID NO: 245 - protein, 231 aa, Artificial Sequence
  VP1 A  subtype amino acid
MDWTWILFLVAAATRVHSTTSAGESADPVTTTVENYGGETQVQRRHHTDVGFIMDRFVKIGNTSPTHVIDLMQTHQHGLVGALLRAATYYFSDLEIVVRHDGNLTWVPNGAPEAALSNTGNPTAYNKAPFTRLALPYTAPHRVLATVYNGTNKYSAASGRTRGDLGTVAARIAAQLPASFNFGAIKADAIHELLVRMKRAELYCPRPLLAVEVSSQDRHKQKIIAPAKQLL

SEQ ID NO: 246 - DNA, 732 bp, Artificial Sequence
  VP1 C  subtype nucleic acid
atggactggacctggattctgtttctcgtggccgctgccacaagagtgcacagcaccaccaccaccggcgagtccgccgacccagtgaccaccaccgtggagaactacggcggcgagacacagacccagcgcaggcaccacacagacgtggccttcgtgctggaccgcttcgtgaaggtgcaggtgtccggcaaccagcacaccctggacgtgatgcaggtgcacaaggactccatcgtgggcgccctgctgagagccgccacctactacttctccgacctggaaatcgccgtgacccacaccggaaagctgacctgggtgcccaatggcgccccagtgtccgccctggacaacaccaccaaccccaccgcctaccacaagggcccactgaccagactggccctgccatacaccgcccctcacagagtgctggccacagcctacaccggcacaaccgcctactccgcctccgccagaagaggcgatctggcccacctcgccgctgcccacgccagacacctgcccaccagcttcaacttcggcgccgtgaaggccgagacaatcaccgagctgctggtgcgcatgaagcgcgccgagctgtactgccccagacccgtgctgccagtgcagccatccggcgacagacacaagcagcccctgatcgccccagccaagcagctgctgtacccctacgacgtgcccgactacgcctccctgggcggaccatgatga

SEQ ID NO: 247 - protein, 228 aa, Artificial Sequence
  VP1 C subtype amino acid
MDWTWILFLVAAATRVHSTTTTGESADPVTTTVENYGGETQTQRRHHTDVAFVLDRFVKVQVSGNQHTLDVMQVHKDSIVGALLRAATYYFSDLEIAVTHTGKLTWVPNGAPVSALDNTTNPTAYHKGPLTRLALPYTAPHRVLATAYTGTTAYSASARRGDLAHLAAAHARHLPTSFNFGAVKAETITELLVRMKRAELYCPRPVLPVQPSGDRHKQPLIAPAKQLL

SEQ ID NO: 248 - DNA, 1404 bp, Artificial Sequence
  VP1 A  subtype + VP1 C subtype nucleic acid
atggactggacctggattctgtttctcgtggccgctgctacaagagtgcactccaccacctctgccggcgagtccgccgacccagtgaccaccaccgtggagaactacggcggcgagacacaggtgcagcgcaggcaccacaccgacgtgggcttcatcatggaccgcttcgtgaagatcggcaacacctcccccacccacgtgatcgacctgatgcagacccaccagcacggactggtgggagccctgctgagagccgccacctactacttctccgacctggaaatcgtggtgcgccacgacggaaacctgacctgggtgcccaatggcgctccagaggccgccctgtccaacaccggcaaccccaccgcctacaacaaggcccccttcaccagactggccctgccatacaccgcccctcacagggtgctggccaccgtgtacaacggcaccaacaagtactccgccgcctccggaagaacaagaggcgacctgggcaccgtggccgccagaatcgccgcccagctgcccgcctccttcaacttcggcgccatcaaggccgacgccatccacgaactgctggtgcgcatgaagcgcgccgagctgtactgcccaagacccctgctggccgtggaggtgtcctcccaggaccgccacaagcagaagatcatcgccccagccaagcagctgctgcgcggcaggaagagaagatccaccaccaccacaggcgaaagcgccgatcccgtgacaacaacagtggaaaattacggcggggaaacccagacccagcggcgccaccacacagatgtggccttcgtgctggacagattcgtgaaggtgcaggtgtccggcaaccagcacaccctggacgtgatgcaggtgcacaaggactccatcgtgggcgccctgctgagggccgccacctactactttagcgatctggaaatcgccgtgacccacaccggcaagctgacatgggtgccaaatggggcccctgtgtctgccctggacaacaccaccaacccaacagcctaccacaaagggccactgacacgcctggccctgccttacacagccccacaccgcgtgctggccacagcctacaccggaaccaccgcctactccgcctccgccagaagaggcgatctggcccatctggccgctgcccacgccagacacctgcccaccagcttcaactttggggccgtgaaagccgagacaatcaccgagctgctggtgcggatgaagagggccgaactgtactgtcctcgccccgtgctgccagtgcagccatccggcgacagacacaagcagcccctgatcgcccctgccaaacagctgctgtacccctacgacgtgcccgactacgcctccctgggcggaccatgatgactcgagtctaga

SEQ ID NO: 249 - protein, 448 aa, Artificial Sequence
  VP1 A  subtype + VP1 C subtype amino acid
MDWTWILFLVAAATRVHSTTSAGESADPVTTTVENYGGETQVQRRHHTDVGFIMDRFVKIGNTSPTHVIDLMQTHQHGLVGALLRAATYYFSDLEIVVRHDGNLTWVPNGAPEAALSNTGNPTAYNKAPFTRLALPYTAPHRVLATVYNGTNKYSAASGRTRGDLGTVAARIAAQLPASFNFGAIKADAIHELLVRMKRAELYCPRPLLAVEVSSQDRHKQKIIAPAKQLLRGRKRRSTTATGESADPVTTTVENYGGETQTQRRHHTDVAFVLDRFVKVQVSGNQHTLDVMQVHKDSIVGALLRAATYYFSDLEIAVTHTGKLTWVPNGAPVSALDNTTNPTAYHKGPLTRLALPYTAPHRVLATAYTGTTAYSASARRGDLAHLAAAHARHLPTSFNFGAVKAETITELLVRMKRAELYCPRPVLPVQPSGDRHKQPLIAPAKQLL